SEQUENCE LISTING (89 sequences)

SEQ ID NO: 1 - protein, 5 aa, synthetic construct
  REGION 1..5: note = Synthetic_H-CDR1 of anti-4-1BB antibody
SYDMS

SEQ ID NO: 2 - protein, 5 aa, synthetic construct
  REGION 1..5: note = Synthetic_H-CDR1 of anti-4-1BB antibody
GYDMS

SEQ ID NO: 3 - protein, 5 aa, synthetic construct
  REGION 1..5: note = Synthetic_H-CDR1 of anti-4-1BB antibody
SYWMH

SEQ ID NO: 4 - protein, 17 aa, synthetic construct
  REGION 1..17: note = Synthetic_H-CDR2 of anti-4-1BB antibody
WISYSGGSIYYADSVKG

SEQ ID NO: 5 - protein, 17 aa, synthetic construct
  REGION 1..17: note = Synthetic_H-CDR2 of anti-4-1BB antibody
VIYPDDGNTYYADSVKG

SEQ ID NO: 6 - protein, 17 aa, synthetic construct
  REGION 1..17: note = Synthetic_H-CDR2 of anti-4-1BB antibody
EINPGNGHTNYNEKFKS

SEQ ID NO: 7 - protein, 12 aa, synthetic construct
  REGION 1..12: note = Synthetic_H-CDR3 of anti-4-1BB antibody
DGQRNSMREFDY

SEQ ID NO: 8 - protein, 12 aa, synthetic construct
  REGION 1..12: note = Synthetic_H-CDR3 of anti-4-1BB antibody
DAQRNSMREFDY

SEQ ID NO: 9 - protein, 12 aa, synthetic construct
  REGION 1..12: note = Synthetic_H-CDR3 of anti-4-1BB antibody
DAQRQSMREFDY

SEQ ID NO: 10 - protein, 18 aa, synthetic construct
  REGION 1..18: note = Synthetic_H-CDR3 of anti-4-1BB antibody
HGGQKPTTKSSSAYGMDG

SEQ ID NO: 11 - protein, 10 aa, synthetic construct
  REGION 1..10: note = Synthetic_H-CDR3 of anti-4-1BB antibody
SFTTARAFAY

SEQ ID NO: 12 - protein, 13 aa, synthetic construct
  REGION 1..13: note = Synthetic_L-CDR1 of anti-4-1BB antibody
SGSSSNIGNNYVT

SEQ ID NO: 13 - protein, 11 aa, synthetic construct
  REGION 1..11: note = Synthetic_L-CDR1 of anti-4-1BB antibody
RASQTISDYLH

SEQ ID NO: 14 - protein, 7 aa, synthetic construct
  REGION 1..7: note = Synthetic_L-CDR2 of anti-4-1BB antibody
ADSHRPS

SEQ ID NO: 15 - protein, 7 aa, synthetic construct
  REGION 1..7: note = Synthetic_L-CDR2 of anti-4-1BB antibody
YASQSIS

SEQ ID NO: 16 - protein, 11 aa, synthetic construct
  REGION 1..11: note = Synthetic_L-CDR3 of anti-4-1BB antibody
ATWDYSLSGYV

SEQ ID NO: 17 - protein, 9 aa, synthetic construct
  REGION 1..9: note = Synthetic_L-CDR3 of anti-4-1BB antibody
QDGHSFPPT

SEQ ID NO: 18 - protein, 121 aa, synthetic construct
  REGION 1..121: note = Synthetic_heavy chain variable region of anti-4-1BB antibody (1A10)
EVQLLESGGGLVQPGGSLRLSCAASGFTFSSYDMSWVRQAPGKGLEWVSWISYSGGSIYYADSVKGRFTISRDNSKNTLYLQMNSLRAEDTAVYYCARDGQRNSMREFDYWGQGTLVTVSS

SEQ ID NO: 19 - protein, 121 aa, synthetic construct
  REGION 1..121: note = Synthetic_heavy chain variable region of anti-4-1BB antibody (1A10 M4, 1A10 M12)
EVQLLESGGGLVQPGGSLRLSCAASGFTFSSYDMSWVRQAPGKGLEWVSWISYSGGSIYYADSVKGRFTISRDNSKNTLYLQMNSLRAEDTAVYYCARDAQRNSMREFDYWGQGTLVTVSS

SEQ ID NO: 20 - protein, 121 aa, synthetic construct
  REGION 1..121: note = Synthetic_heavy chain variable region of anti-4-1BB antibody (1A10 M11, 1A10 M13)
EVQLLESGGGLVQPGGSLRLSCAASGFTFSSYDMSWVRQAPGKGLEWVSWISYSGGSIYYADSVKGRFTISRDNSKNTLYLQMNSLRAEDTAVYYCARDAQRQSMREFDYWGQGTLVTVSS

SEQ ID NO: 21 - protein, 127 aa, synthetic construct
  REGION 1..127: note = Synthetic_heavy chain variable region of anti-4-1BB antibody (1A12)
EVQLLESGGGLVQPGGSLRLSCAASGFTFSGYDMSWVRQAPGKGLEWVSVIYPDDGNTYYADSVKGRFTISRDNSKNTLYLQMNSLRAEDAAVYYCAKHGGQKPTTKSSSAYGMDGWGQGTLVTVSS

SEQ ID NO: 22 - protein, 127 aa, synthetic construct
  REGION 1..127: note = Synthetic_heavy chain variable region of anti-4-1BB antibody (1A12M1)
EVQLLESGGGLVQPGGSLRLSCAASGFTFSGYDMSWVRQAPGKGLEWVSVIYPDDGNTYYADSVKGRFTISRDNSKNTLYLQMNSLRAEDTAVYYCAKHGGQKPTTKSSSAYGMDGWGQGTLVTVSS

SEQ ID NO: 23 - protein, 119 aa, synthetic construct
  REGION 1..119: note = Synthetic_heavy chain variable region of anti-4-1BB antibody (AB41)
QVQLQQSGAEVIKPGASVKLSCKASGYTFSSYWMHWVRQAPGQGLEWIGEINPGNGHTNYNEKFKSRATLTGDTSTSTVYMELSSLRSEDTAVYYCARSFTTARAFAYWGQGTLVTVSS

SEQ ID NO: 24 - protein, 121 aa, synthetic construct
  REGION 1..121: note = Synthetic_heavy chain variable region of anti-4-1BB antibody (mutated 1A10)
EVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDGQRNSMREFDYWGQGTLVTVSS

SEQ ID NO: 25 - protein, 121 aa, synthetic construct
  REGION 1..121: note = Synthetic_heavy chain variable region of anti-4-1BB antibody (mutated 1A10 M4, 1A10 M12)
EVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 26 - protein, 121 aa, synthetic construct
  REGION 1..121: note = Synthetic_heavy chain variable region of anti-4-1BB antibody (mutated 1A10 M11, 1A10 M13)
EVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRQSMREFDYWGQGTLVTVSS

SEQ ID NO: 27 - protein, 127 aa, synthetic construct
  REGION 1..127: note = Synthetic_heavy chain variable region of anti-4-1BB antibody (mutated 1A12)
EVQLLESGGGLVQPGGSLRLSCAASGFTFSGYDMSWVRQAPGKCLEWVSVIYPDDGNTYYADSVKGRFTISRDNSKNTLYLQMNSLRAEDAAVYYCAKHGGQKPTTKSSSAYGMDGWGQGTLVTVSS

SEQ ID NO: 28 - protein, 127 aa, synthetic construct
  REGION 1..127: note = Synthetic_heavy chain variable region of anti-4-1BB antibody (mutated 1A12M1)
EVQLLESGGGLVQPGGSLRLSCAASGFTFSGYDMSWVRQAPGKCLEWVSVIYPDDGNTYYADSVKGRFTISRDNSKNTLYLQMNSLRAEDTAVYYCAKHGGQKPTTKSSSAYGMDGWGQGTLVTVSS

SEQ ID NO: 29 - protein, 119 aa, synthetic construct
  REGION 1..119: note = Synthetic_heavy chain variable region of anti-4-1BB antibody (mutated AB41)
QVQLQQSGAEVIKPGASVKLSCKASGYTFSSYWMHWVRQAPGQCLEWIGEINPGNGHTNYNEKFKSRATLTGDTSTSTVYMELSSLRSEDTAVYYCARSFTTARAFAYWGQGTLVTVSS

SEQ ID NO: 30 - protein, 110 aa, synthetic construct
  REGION 1..110: note = Synthetic_light chain variable region of anti-4-1BB antibody (1A10, 1A10 M4, 1A10 M11, 1A12)
QSVLTQPPSASGTPGRRVTISCSGSSSNIGNNYVTWYQQLPGTAPKLLIYADSHRPSGVPDRFSGSKSGTSASLAISGLRSEDEADYYCATWDYSLSGYVFGGGTKLTVL

SEQ ID NO: 31 - protein, 110 aa, synthetic construct
  REGION 1..110: note = Synthetic_light chain variable region of anti-4-1BB antibody (1A10 M12, 1A10 M13, 1A12M1)
QSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGGGTKLTVL

SEQ ID NO: 32 - protein, 108 aa, synthetic construct
  REGION 1..108: note = Synthetic_light chain variable region of anti-4-1BB antibody (AB41)
DIVMTQSPAFLSVTPGEKVTITCRASQTISDYLHWYQQKPDQAPKLLIKYASQSISGIPSRFSGSGSGTDFTFTISSLEAEDAATYYCQDGHSFPPTFGQGTKLEIKR

SEQ ID NO: 33 - protein, 110 aa, synthetic construct
  REGION 1..110: note = Synthetic_light chain variable region of anti-4-1BB antibody (mutated 1A10, 1A10 M4, 1A10 M11, 1A12)
QSVLTQPPSASGTPGRRVTISCSGSSSNIGNNYVTWYQQLPGTAPKLLIYADSHRPSGVPDRFSGSKSGTSASLAISGLRSEDEADYYCATWDYSLSGYVFGCGTKLTVL

SEQ ID NO: 34 - protein, 110 aa, synthetic construct
  REGION 1..110: note = Synthetic_light chain variable region of anti-4-1BB antibody (mutated 1A10 M12, 1A10 M13, 1A12M1)
QSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVL

SEQ ID NO: 35 - protein, 30 aa, synthetic construct
  REGION 1..30: note = Synthetic_H-FR1 of anti-4-1BB antibody
EVQLLESGGGLVQPGGSLRLSCAASGFTFS

SEQ ID NO: 36 - protein, 30 aa, synthetic construct
  REGION 1..30: note = Synthetic_H-FR1 of anti-4-1BB antibody
QVQLQQSGAEVIKPGASVKLSCKASGYTFS

SEQ ID NO: 37 - protein, 14 aa, synthetic construct
  REGION 1..14: note = Synthetic_H-FR2 of anti-4-1BB antibody
WVRQAPGKGLEWVS

SEQ ID NO: 38 - protein, 14 aa, synthetic construct
  REGION 1..14: note = Synthetic_H-FR2 of anti-4-1BB antibody
WVRQAPGQGLEWIG

SEQ ID NO: 39 - protein, 14 aa, synthetic construct
  REGION 1..14: note = Synthetic_H-FR2 of anti-4-1BB antibody
WVRQAPGKCLEWVS

SEQ ID NO: 40 - protein, 14 aa, synthetic construct
  REGION 1..14: note = Synthetic_H-FR2 of anti-4-1BB antibody
WVRQAPGQCLEWIG

SEQ ID NO: 41 - protein, 32 aa, synthetic construct
  REGION 1..32: note = Synthetic_H-FR3 of anti-4-1BB antibody
RFTISRDNSKNTLYLQMNSLRAEDTAVYYCAR

SEQ ID NO: 42 - protein, 32 aa, synthetic construct
  REGION 1..32: note = Synthetic_H-FR3 of anti-4-1BB antibody
RFTISRDNSKNTLYLQMNSLRAEDAAVYYCAK

SEQ ID NO: 43 - protein, 32 aa, synthetic construct
  REGION 1..32: note = Synthetic_H-FR3 of anti-4-1BB antibody
RFTISRDNSKNTLYLQMNSLRAEDTAVYYCAK

SEQ ID NO: 44 - protein, 32 aa, synthetic construct
  REGION 1..32: note = Synthetic_H-FR3 of anti-4-1BB antibody
RATLTGDTSTSTVYMELSSLRSEDTAVYYCAR

SEQ ID NO: 45 - protein, 11 aa, synthetic construct
  REGION 1..11: note = Synthetic_H-FR4 of anti-4-1BB antibody
WGQGTLVTVSS

SEQ ID NO: 46 - protein, 22 aa, synthetic construct
  REGION 1..22: note = Synthetic_L-FR1 of anti-4-1BB antibody
QSVLTQPPSASGTPGRRVTISC

SEQ ID NO: 47 - protein, 22 aa, synthetic construct
  REGION 1..22: note = Synthetic_L-FR1 of anti-4-1BB antibody
QSVLTQPPSASGTPGQRVTISC

SEQ ID NO: 48 - protein, 23 aa, synthetic construct
  REGION 1..23: note = Synthetic_L-FR1 of anti-4-1BB antibody
DIVMTQSPAFLSVTPGEKVTITC

SEQ ID NO: 49 - protein, 15 aa, synthetic construct
  REGION 1..15: note = Synthetic_L-FR2 of anti-4-1BB antibody
WYQQLPGTAPKLLIY

SEQ ID NO: 50 - protein, 15 aa, synthetic construct
  REGION 1..15: note = Synthetic_L-FR2 of anti-4-1BB antibody
WYQQKPDQAPKLLIK

SEQ ID NO: 51 - protein, 32 aa, synthetic construct
  REGION 1..32: note = Synthetic_L-FR3 of anti-4-1BB antibody
GVPDRFSGSKSGTSASLAISGLRSEDEADYYC

SEQ ID NO: 52 - protein, 32 aa, synthetic construct
  REGION 1..32: note = Synthetic_L-FR3 of anti-4-1BB antibody
GIPSRFSGSGSGTDFTFTISSLEAEDAATYYC

SEQ ID NO: 53 - protein, 10 aa, synthetic construct
  REGION 1..10: note = Synthetic_L-FR4 of anti-4-1BB antibody
FGGGTKLTVL

SEQ ID NO: 54 - protein, 11 aa, synthetic construct
  REGION 1..11: note = Synthetic_L-FR4 of anti-4-1BB antibody
FGQGTKLEIKR

SEQ ID NO: 55 - protein, 10 aa, synthetic construct
  REGION 1..10: note = Synthetic_L-FR4 of anti-4-1BB antibody
FGCGTKLTVL

SEQ ID NO: 56 - protein, 448 aa, synthetic construct
  REGION 1..448: note = Synthetic_heavy chain of anti-4-1BB antibody
EVQLLESGGGLVQPGGSLRLSCAASGFTFSSYDMSWVRQAPGKGLEWVSWISYSGGSIYYADSVKGRFTISRDNSKNTLYLQMNSLRAEDTAVYYCARDGQRNSMREFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 448 aa, synthetic construct
  REGION 1..448: note = Synthetic_heavy chain of anti-4-1BB antibody
EVQLLESGGGLVQPGGSLRLSCAASGFTFSSYDMSWVRQAPGKGLEWVSWISYSGGSIYYADSVKGRFTISRDNSKNTLYLQMNSLRAEDTAVYYCARDAQRNSMREFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 58 - protein, 448 aa, synthetic construct
  REGION 1..448: note = Synthetic_heavy chain of anti-4-1BB antibody
EVQLLESGGGLVQPGGSLRLSCAASGFTFSSYDMSWVRQAPGKGLEWVSWISYSGGSIYYADSVKGRFTISRDNSKNTLYLQMNSLRAEDTAVYYCARDAQRQSMREFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 454 aa, synthetic construct
  REGION 1..454: note = Synthetic_heavy chain of anti-4-1BB antibody
EVQLLESGGGLVQPGGSLRLSCAASGFTFSGYDMSWVRQAPGKGLEWVSVIYPDDGNTYYADSVKGRFTISRDNSKNTLYLQMNSLRAEDAAVYYCAKHGGQKPTTKSSSAYGMDGWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - protein, 454 aa, synthetic construct
  REGION 1..454: note = Synthetic_heavy chain of anti-4-1BB antibody
EVQLLESGGGLVQPGGSLRLSCAASGFTFSGYDMSWVRQAPGKGLEWVSVIYPDDGNTYYADSVKGRFTISRDNSKNTLYLQMNSLRAEDTAVYYCAKHGGQKPTTKSSSAYGMDGWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 61 - protein, 446 aa, synthetic construct
  REGION 1..446: note = Synthetic_heavy chain of anti-4-1BB antibody
QVQLQQSGAEVIKPGASVKLSCKASGYTFSSYWMHWVRQAPGQGLEWIGEINPGNGHTNYNEKFKSRATLTGDTSTSTVYMELSSLRSEDTAVYYCARSFTTARAFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 62 - protein, 216 aa, synthetic construct
  REGION 1..216: note = Synthetic_light chain of anti-4-1BB antibody
QSVLTQPPSASGTPGRRVTISCSGSSSNIGNNYVTWYQQLPGTAPKLLIYADSHRPSGVPDRFSGSKSGTSASLAISGLRSEDEADYYCATWDYSLSGYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 63 - protein, 216 aa, synthetic construct
  REGION 1..216: note = Synthetic_light chain of anti-4-1BB antibody
QSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 64 - protein, 214 aa, synthetic construct
  REGION 1..214: note = Synthetic_light chain of anti-4-1BB antibody
DIVMTQSPAFLSVTPGEKVTITCRASQTISDYLHWYQQKPDQAPKLLIKYASQSISGIPSRFSGSGSGTDFTFTISSLEAEDAATYYCQDGHSFPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 5 aa, synthetic construct
  REGION 1..5: note = Synthetic_H-CDR1 of anti-HER2 antibody
DTYIH

SEQ ID NO: 66 - protein, 17 aa, synthetic construct
  REGION 1..17: note = Synthetic_H-CDR2 of anti-HER2 antibody
RIYPTNGYTRYADSVKG

SEQ ID NO: 67 - protein, 11 aa, synthetic construct
  REGION 1..11: note = Synthetic_H-CDR3 of anti-HER2 antibody
WGGDGFYAMDY

SEQ ID NO: 68 - protein, 11 aa, synthetic construct
  REGION 1..11: note = Synthetic_L-CDR1 of anti-HER2 antibody
RASQDVNTAVA

SEQ ID NO: 69 - protein, 7 aa, synthetic construct
  REGION 1..7: note = Synthetic_L-CDR2 of anti-HER2 antibody
SASFLYS

SEQ ID NO: 70 - protein, 9 aa, synthetic construct
  REGION 1..9: note = Synthetic_L-CDR3 of anti-HER2 antibody
QQHYTTPPT

SEQ ID NO: 71 - protein, 119 aa, synthetic construct
  REGION 1..119: note = Synthetic_heavy chain variable region of anti-HER2 antibody
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVS

SEQ ID NO: 72 - protein, 108 aa, synthetic construct
  REGION 1..108: note = Synthetic_light chain variable region of anti-HER2 antibody
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKR

SEQ ID NO: 73 - protein, 450 aa, synthetic construct
  REGION 1..450: note = Synthetic_heavy chain of anti-HER2 antibody
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 450 aa, synthetic construct
  REGION 1..450: note = Synthetic_heavy chain of anti-HER2 antibody (N297A)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 214 aa, synthetic construct
  REGION 1..214: note = Synthetic_light chain of anti-HER2 antibody
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 716 aa, synthetic construct
  REGION 1..716: note = Synthetic_heavy chain of bispecific antibody (HER2(NA)x1A10)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQSVLTQPPSASGTPGRRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDGQRNSMREFDYWGQGTLVTVSS

SEQ ID NO: 77 - protein, 719 aa, synthetic construct
  REGION 1..719: note = Synthetic_heavy chain of bispecific antibody (HER2 (NA)x1A10 GSLH)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGSGSGSGSGSGSGSGSQSVLTQPPSASGTPGRRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDGQRNSMREFDYWGQGTLVTVSS

SEQ ID NO: 78 - protein, 719 aa, synthetic construct
  REGION 1..719: note = Synthetic_heavy chain of bispecific antibody (HER2(NA)x1A10 M4)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGSGSGSGSGSGSGSGSQSVLTQPPSASGTPGRRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 79 - protein, 719 aa, synthetic construct
  REGION 1..719: note = Synthetic_heavy chain of bispecific antibody(HER2(NA)x1A10 M12)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGSGSGSGSGSGSGSGSQSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 80 - protein, 722 aa, synthetic construct
  REGION 1..722: note = Synthetic_heavy chain of bispecific antibody (HER2(NA)x1A12)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQSVLTQPPSASGTPGRRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSGYDMSWVRQAPGKCLEWVSVIYPDDGNTYYADSVKGRFTISRDNSKNTLYLQMNSLRAEDAAVYYCAKHGGQKPTTKSSSAYGMDGWGQGTLVTVSS

SEQ ID NO: 81 - protein, 725 aa, synthetic construct
  REGION 1..725: note = Synthetic_heavy chain of bispecific antibody (HER2 (NA)x1A12 GSLH)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGSGSGSGSGSGSGSGSQSVLTQPPSASGTPGRRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSGYDMSWVRQAPGKCLEWVSVIYPDDGNTYYADSVKGRFTISRDNSKNTLYLQMNSLRAEDAAVYYCAKHGGQKPTTKSSSAYGMDGWGQGTLVTVSS

SEQ ID NO: 82 - protein, 725 aa, synthetic construct
  REGION 1..725: note = Synthetic_heavy chain of bispecific antibody (HER2(NA)x1A12 M1)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGSGSGSGSGSGSGSGSQSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSGYDMSWVRQAPGKCLEWVSVIYPDDGNTYYADSVKGRFTISRDNSKNTLYLQMNSLRAEDTAVYYCAKHGGQKPTTKSSSAYGMDGWGQGTLVTVSS

SEQ ID NO: 83 - protein, 719 aa, synthetic construct
  REGION 1..719: note = Synthetic_heavy chain of bispecific antibody(HER2(WT)x1A10 M12)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGSGSGSGSGSGSGSGSQSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 84 - protein, 725 aa, synthetic construct
  REGION 1..725: note = Synthetic_heavy chain of bispecific antibody (HER2(WT)x1A12)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGSGSGSGSGSGSGSGSQSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSGYDMSWVRQAPGKCLEWVSVIYPDDGNTYYADSVKGRFTISRDNSKNTLYLQMNSLRAEDTAVYYCAKHGGQKPTTKSSSAYGMDGWGQGTLVTVSS

SEQ ID NO: 85 - protein, 15 aa, synthetic construct
  REGION 1..15: note = Synthetic_peptide linker
GGGGSGGGGSGGGGS

SEQ ID NO: 86 - protein, 18 aa, synthetic construct
  REGION 1..18: note = Synthetic_peptide linker
GSGSGSGSGSGSGSGSGS

SEQ ID NO: 87 - protein, 20 aa, synthetic construct
  REGION 1..20: note = Synthetic_peptide linker
GGGGSGGGGSGGGGSGGGGS

SEQ ID NO: 88 - protein, 108 aa, synthetic construct
  REGION 1..108: note = Synthetic_light chain variable region of anti-4-1BB antibody (mutated AB41)
DIVMTQSPAFLSVTPGEKVTITCRASQTISDYLHWYQQKPDQAPKLLIKYASQSISGIPSRFSGSGSGTDFTFTISSLEAEDAATYYCQDGHSFPPTFGCGTKLEIKR

SEQ ID NO: 89 - protein, 255 aa, synthetic construct
  REGION 1..255: note = Synthetic_human 4-1BB (NP_001552.2)
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

(19) United States
(12) Patent Application Publication
(10) Pub. No.: US 2025/0163180 A1
(43) Pub. Date: May 22, 2025

(54) ANTI-HER2/ANTI-4-1BB BISPECIFIC ANTIBODY AND USE THEREOF

(71) Applicants: ABL Bio Inc., Seongnam-si (KR); YUHAN CORPORATION, Seoul (KR)

(72) Inventors: Hyejin CHUNG, Seongnam-si (KR); Yeryoung YONG, Seongnam-si (KR); Kyeongsu PARK, Seongnam-si (KR); Eunyoung PARK, Seongnam-si (KR); Ui-Jung JUNG, Seongnam-si (KR); Yangsoon LEE, Seongnam-si (KR); Eunjung KIM, Seongnam-si (KR); Yong-Gyu SON, Seongnam-si (KR); Wonjun SON, Seongnam-si (KR); Seawon AHN, Seongnam-si (KR); Donghoon YEOM, Seongnam-si (KR); Chanmoo LEE, Gwacheon-si (KR); Junghyeon HONG, Seongnam-si (KR); Moo Young SONG, Suwon-si (KR); Eun-Jung LEE, Yongin-si (KR); Na Rae LEE, Seoul (KR); Young Bong PARK, Yongin-si (KR); Eun-Jung LEE, Yongin-si (KR); Taewang KIM, Yongin-si (KR)

(21) Appl. No.: 19/034,755

(22) Filed: Jan. 23, 2025

Related U.S. Application Data
(62) Division of application No. 16/939,536, filed on Jul. 27, 2020, now Pat. No. 12,240,913.
(60) Provisional application No. 63/024,608, filed on May 14, 2020.
(60) Provisional application No. 62/878,951, filed on Jul. 26, 2019.

(51) Int. Cl. C07K 16/32; A61K 39/00; A61P 35/00; C07K 16/28

(52) U.S. Cl. CPC C07K 16/32; A61P 35/00; C07K 16/2878; A61K 2039/505; C07K 2317/21; C07K 2317/31; C07K 2317/51; C07K 2317/515; C07K 2317/56; C07K 2317/565; C07K 2317/622; C07K 2317/92

(57) ABSTRACT

Provided are an anti-4-1BB/anti-HER2 bispecific antibody, and a pharmaceutical composition and a method for treating and/or preventing a cancer using the same.

### CROSS-REFERENCE TO RELATED APPLICATIONS

This application is a divisional application of U.S. Ser. No. 16/939,536 filed on Jul. 27, 2020, which claims the benefit of U.S. 62/878,951 filed on Jul. 26, 2019 and U.S. 63/024,608 filed on May 14, 2020, with the United States Patent and Trademark Office, the entire disclosure of which is herein incorporated by reference.

### REFERENCE TO AN ELECTRONIC SEQUENCE LISTING

The contents of the electronic sequence listing (LPP20250264US_SEQ.xml; Size: 128,068 bytes; and Date of Creation: Jan. 23, 2025) is herein incorporated by reference in its entirety. The contents of the electronic sequence listing in no way introduces new matter into the specification.

### BACKGROUND OF THE INVENTION

### 1. Field

Provided are an anti-4-1BB/anti-HER2 bispecific antibody, and a pharmaceutical composition and a method for treating and/or preventing a cancer using the same.

### 2. Description of the Related Art

4-1BB protein is a member of TNF-receptor superfamily (TNFRSF) and is a co-stimulatory molecule which is expressed following the activation of immune cells, both innate and adaptive immune cells. 4-1BB plays important role in modulate the activity of various immune cells. 4-1BB agonists enhance proliferation and survival of immune cells, secretion of cytokines, and cytolytic activity CD8 T cells. Many other studies showed that activation of 4-1BB enhances immune response to eliminate tumors in mice. Therefore, it suggests that 4-1BB is a promising target molecule in cancer immunology. Despite of their anti-tumor efficacy, anti-4-1BB antibody induced severe liver toxicity in clinical application.

HER2 protein is a member of epidermal growth factor receptor (EGFR) family and involved in various mechanisms related to tumors. HER2 is a typical receptor tyrosine kinase (RTK) present on the surface of cells, and thereby inducing proliferation and penetration of cancer cells, angiogenesis, etc.

Meanwhile, multispecific antibodies targeting two or more antigens have been developed in various kinds and forms and are expected as a new drug antibody having excellent therapeutic effects compared to a monoclonal antibody.

Therefore, there is a need to develop a multispecific antibody capable of recognizing two different antigens wherein one is present on a cancer cell and the other is present on other cell such as an immune cell, for more efficient cancer therapy.

### SUMMARY OF THE INVENTION

One embodiment provides an anti-HER2/anti-4-1BB bispecific antibody, comprising:
(1) an anti-HER2 antibody or an antigen-binding fragment thereof, as a HER2 targeting moiety, which is capable of specifically recognizing and/or binding to HER2 protein; and
(2) an anti-4-1BB antibody or an antigen-binding fragment thereof, as a 4-1BB targeting moiety, which is capable of specifically recognizing and/or binding to 4-1BB protein.

Another embodiment provides a pharmaceutical composition comprising the bispecific antibody. The pharmaceutical composition may further comprise a pharmaceutically acceptable carrier. The pharmaceutical composition may be used for treating and/or preventing a cancer and/or for enhancing immune response.

Another embodiment provides a pharmaceutical composition for treating and/or preventing a cancer and/or for enhancing immune response, the composition comprising the bispecific antibody.

Another embodiment provides a method of treating and/or preventing a cancer in a subject in need thereof, comprising administering to the subject a pharmaceutically effective amount of the bispecific antibody or the pharmaceutical composition to the subject. The method may further comprise a step of identifying the subject in need of treating and/or preventing a cancer, prior to the administering step.

Another embodiment provides a method of enhancing immune response in a subject in need thereof, comprising administering to the subject a pharmaceutically effective amount of the bispecific antibody or the pharmaceutical composition to the subject. The method may further comprise a step of identifying the subject in need of enhancing immune response, prior to the administering step.

Another embodiment provides a use of the bispecific antibody or the pharmaceutical composition in treating and/or preventing a cancer. Another embodiment provides a use of the bispecific antibody in preparing a medicament for treating and/or preventing a cancer.

Another embodiment provides a use of the bispecific antibody or the pharmaceutical composition in enhancing immune response. Another embodiment provides a use of the bispecific antibody in preparing a medicament for enhancing immune response.

An embodiment provides a polynucleotide encoding the bispecific antibody.

An embodiment provides a recombinant vector comprising the polynucleotide. The recombinant vector may be used as an expression vector of a polynucleotide encoding the bispecific antibody.

Another embodiment provides a cell comprising a polynucleotide encoding the bispecific antibody. The cell may be a recombinant cell transfected with a recombinant vector comprising the polynucleotide.

Another embodiment provides a method of preparing the bispecific antibody, comprising expressing the polynucleotide in a cell. The step of expressing the polynucleotide may be conducted by culturing the cell comprising the polynucleotide (for example, in a recombinant vector) under a condition allowing the expression of the polynucleotide.

### BRIEF DESCRIPTION OF THE DRAWINGS

FIG. 1a is a graph showing antigen (human 4-1BB) binding activities of anti-4-1BB antibodies measured by ELISA.

FIG. 1b is a graph showing cell binding activities of anti-4-1BB antibodies measured by ELISA.

FIG. 2a is a graph showing antigen (human HER2) binding activities of anti-HER2/anti-4-1BB bispecific antibodies measured by ELISA.

FIG. 2b is a graph showing antigen (human HER2) binding activities of anti-HER2/anti-4-1BB bispecific antibodies measured by ELISA.

FIG. 3a is a graph showing antigen (human 4-1BB) binding activities of anti-HER2/anti-4-1BB bispecific antibodies measured by ELISA.

FIG. 3b is a graph showing antigen (human 4-1BB) binding activities of anti-HER2/anti-4-1BB bispecific antibodies measured by ELISA.

FIG. 4a is a graph showing 4-1BB signal activation level by anti-HER2/anti-4-1BB bispecific antibodies in NCI-N87 cell line (HER2 high expressing cells).

FIG. 4b is a graph showing 4-1BB signal activation level by anti-HER2/anti-4-1BB bispecific antibodies in MDA-MB-231 cell line (HER2 negative cells).

FIG. 5a is a graph showing 4-1BB signal activation level by an anti-HER2/anti-4-1BB bispecific antibody in HER2-expressing NCI-N87 cell line.

FIG. 5b is a graph showing 4-1BB signal activation level by an anti-HER2/anti-4-1BB bispecific antibody in HER2-expressing Calu-3 cell line.

FIG. 5c is a graph showing 4-1BB signal activation level by an anti-HER2/anti-4-1BB bispecific antibody in HER2-expressing HCC1954 cell line.

FIG. 5d is a graph showing 4-1BB signal activation level by an anti-HER2/anti-4-1BB bispecific antibody in HER2-expressing JIMT1 cell line.

FIG. 5e is a graph showing 4-1BB signal activation level by an anti-HER2/anti-4-1BB bispecific antibody in HER2-expressing ZR-75-1 cell line.

FIG. 5f is a graph showing 4-1BB signal activation level by an anti-HER2/anti-4-1BB bispecific antibody in HER2-non-expressing A431 cell line.

FIG. 5g is a graph showing 4-1BB signal activation level by an anti-HER2/anti-4-1BB bispecific antibody in HER2-non-expressing MCF-7 cell line.

FIG. 5h is a graph showing 4-1BB signal activation level by an anti-HER2/anti-4-1BB bispecific antibody in HER2-non-expressing MDA-MB231 cell line.

FIG. 5i is a graph showing 4-1BB signal activation level by an anti-HER2/anti-4-1BB bispecific antibody in HER2-non-expressing BxPC-3 cell line.

FIG. 6a is a graph showing 4-1BB signal activation level by anti-HER2/anti-4-1BB bispecific antibodies in HER2-expressing Calu-3 cell line.

FIG. 6b is a graph showing 4-1BB signal activation level by anti-HER2/anti-4-1BB bispecific antibodies in HER2-expressing HCC1954 cell line.

FIG. 7 is a graph showing correlation between the HER2 sABC and 4-1BB-induced NF-kB signaling by an anti-HER2/anti-4-1BB bispecific antibody in various cell lines.

FIGS. 8a-8d are graphs showing IFN-gamma level released from HER2-expressing HCC1954 cells treated with anti-HER2/anti-4-1BB bispecific antibodies.

FIGS. 9a-9d are graphs showing % survival of HER2-expressing HCC1954 cells treated with anti-HER2/anti-4-1BB bispecific antibodies.

FIG. 10 is a graph showing in vivo anti-tumor activities by anti-HER2/anti-4-1BB bispecific antibodies in HCC1954 bearing hPBMC engrafted mice.

FIG. 11 is a graph showing lymphocytes marker positive cell counts in HCC1954 bearing hPBMC engrafted mice treated with anti-HER2/anti-4-1BB bispecific antibodies.

FIG. 12 is a graph showing in vivo anti-tumor activities by anti-HER2/anti-4-1BB bispecific antibodies in human HER2/MC38 tumor bearing 4-1BB knock-in mice.

FIG. 13 is a graph showing in vivo anti-tumor activities by anti-HER2/anti-4-1BB bispecific antibodies in mice cured by anti-HER2/anti-4-1BB bispecific antibody and re-challenged with human HER2/MC38 tumor cells and B16 F10 tumor cells.

FIG. 14 is a graph showing antibody-dependent cellular cytotoxicity (ADCC) effects of anti-HER2/anti-4-1BB bispecific antibodies.

FIG. 15a is a graph showing results of FcγRIIb-dependent 4-1BB bioassay for anti-HER2/anti-4-1BB bispecific antibodies.

FIG. 15b is a graph showing results of FcγRIIb-independent 4-1BB bioassay for anti-HER2/anti-4-1BB bispecific antibodies.

### DETAILED DESCRIPTION OF THE INVENTION

The present disclosure relates to bispecific antibodies, each of which comprises an antibody specific to a tumor associated antigen (TAA; HER2) and an antibody specific to 4-1BB, and uses thereof. These bispecific antibodies activate 4-1BB signaling and boost strong immune cell only in presence of HER2 expressing cells. Due to specific HER2-mediated immune response, it is expected to have much less liver toxicity by using bispecific antibodies compared to 4-1BB monoclonal antibody.

In the present disclosure, provided is an anti-HER2/anti-4-1BB bispecific antibody, and uses thereof, wherein the anti-HER2/anti-4-1BB bispecific antibody may comprise:
(1) an anti-HER2 antibody or an antigen-binding fragment thereof, as a HER2 targeting moiety, which is capable of specifically recognizing and/or binding to HER2 protein, and
(2) an anti-4-1BB antibody or an antigen-binding fragment thereof, as a 4-1BB targeting moiety, which is capable of specifically recognizing and/or binding to 4-1BB protein.

Hereinafter, the present invention is described in more detail.

### Definition

As used herein, ‘consisting of a sequence,’ ‘consisting essentially of a sequence,’ or ‘comprising a sequence’ may refer to any case comprising the sequence, but it may not be intended to exclude a case comprising further sequence other than the sequence.

As used herein, the term ‘a protein or polypeptide comprising or consisting of an amino acid sequence identified by SEQ ID NO’ and ‘a gene or polynucleotide comprising or consisting of a nucleic acid sequence identified by SEQ ID NO’ may refer to a protein (or polypeptide) or gene (or polynucleotide), which consists essentially of the amino acid sequence or nucleic acid sequence, or which has at least 90%, at least 91%, at least 92%, at least 93%, at least 94%, at least 95%, at least 96%, at least 97%, at least 98%, or at least 99% sequence identity with the amino acid sequence or nucleic acid sequence with maintaining its inherent activity and/or function.

As used herein, the term “antibody” may encompass various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4), and light chains are classified as either kappa or lambda (K, λ). It is the nature of this chain that determines the “class” of the antibody as IgG, IgM, IgA IgG, or IgE, respectively. The immunoglobulin subclasses (isotypes), e.g., IgG1, IgG2, IgG3, IgG4, IgG5, etc., are well characterized and are known to confer functional specialization.

An intact antibody includes two full-length light chains and two full-length heavy chains, in which each light chain is linked to a heavy chain by disulfide bonds. The antibody has a heavy chain constant region and a light chain constant region. The heavy chain constant region is of a gamma (γ), mu (μ), alpha (α), delta (δ), or epsilon (ε) type, which may be further categorized as gamma 1 (γ1), gamma 2(γ2), gamma 3(γ3), gamma 4(γ4), alpha 1(α1), or alpha 2(α2). The light chain constant region is of either a kappa (κ) or lambda (λ) type.

The term “heavy chain” refers to a full-length heavy chain or a fragment thereof, including a variable region V_{H} that includes amino acid sequences sufficient to provide specificity to antigens, and three constant regions, C_{H1}, C_{H2}, and C_{H3}, and a hinge. The term “light chain” refers to a full-length light chain or a fragment thereof, including a variable region V_{L} that includes amino acid sequences sufficient to provide specificity to antigens, and a constant region C_{L}.

The term “complementarity determining region (CDR)” refers to an amino acid sequence found in a hyper variable region of a heavy chain or a light chain of immunoglobulin. The heavy and light chains may respectively include three CDRs (CDRH1, CDRH2, and CDRH3; and CDRL1, CDRL2, and CDRL3). The CDR may provide residues that play an important role in the binding of antibodies to an antigens or epitope. The terms “specifically binding” or “specifically recognized” is well known to one of ordinary skill in the art, and indicates that an antibody and an antigen specifically interact with each other to lead to an immunological activity.

In this disclosure, the antibody may include, but not be limited to, polyclonal or monoclonal; and/or human, humanized, animal (e.g., mouse, rabbit, etc.) derived antibody, or chimeric antibodies (e.g., mouse-human chimeric antibody).

An animal-derived antibody which is produced by immunizing an animal with a desired antigen may generally trigger an immune rejection response when administered to humans for treatment purpose, and a chimeric antibody has been developed to suppress such immune rejection response. A chimeric antibody is formed by replacing the constant region of an animal-derived antibody, which is a cause of anti-isotype response, with the constant region of a human antibody using genetic engineering methods. The chimeric antibody has considerably improved anti-isotype response in comparison with animal-derived antibodies, but animal-derived amino acids are still present in its variable regions and thus it still contains potential side effects resulting from an anti-idiotypic response. It is a humanized antibody that has been thus developed to improve such side effects. This is manufactured by grafting CDR (complementarity determining regions) which, of the variable regions of a chimeric antibody, has an important role in antigen binding into a human antibody framework.

As used herein, the term “antigen binding fragment” refers to a fragment derived from a full immunoglobulin structure including a portion capable of binding to an antigen such as CDRs. For example, the antigen binding fragment may be scFv, (scFv)₂, Fab, Fab′, or F(ab′)₂, but not be limited thereto. In the present disclosure, the antigen binding fragment may be a fragment derived from an antibody, including at least one complementarity determining region, for example, selected from the group consisting of scFv, (scFv)2, scFv-Fc, Fab, Fab′ and F(ab′)2.

Of the antigen binding fragments, Fab is a structure having variable regions of a light chain and a heavy chain, a constant region of the light chain, and the first constant region (C_{H1}) of the heavy chain, and it has one antigen binding site.

Fab′ is different from Fab in that it has a hinge region including one or more cysteine residues at the C-terminal of heavy chain CHI domain. An F(ab′)₂ antibody is formed through disulfide bond of the cysteine residues at the hinge region of Fab′.

Fv is a minimal antibody piece having only a heavy chain variable region and light chain variable region, and a recombinant technique for producing the Fv fragment is well known in the pertinent art. Two-chain Fv may have a structure in which the heavy chain variable region is linked to the light chain variable region by a non-covalent bond, and single-chain Fv (scFv) may generally have a dimer structure as in the two-chain Fv in which the variable region of a heavy chain and the variable region of a light chain are covalently linked via a peptide linker or they are directly linked to each other at the C-terminal thereof.

The antigen binding fragments may be obtained using proteases (for example, a whole antibody is digested with papain to obtain Fab fragments, and is digested with pepsin to obtain F(ab′)₂ fragments), and may be prepared by a genetic recombinant technique.

Immunoglobulin (e.g., a human immunoglobulin) or antibody molecules of the disclosure can be of any type (e.g., IgG, IgE, IgM, IgD, IgA, IgY, etc.), class (e.g., IgG1, IgG2, IgG3, IgG4, IgG5, IgA1, IgA2, etc.), or subclass of immunoglobulin molecule.

In the antibody or antibody fragment, portions (e.g., constant regions) except the CDRs or variable regions may be derived from a human antibody and particularly, they may be derived from IgG, IgA, IgD, IgE, IgM, or IgY, for example, IgG1, IgG2, IgG3, or IgG4.

The antibody or antigen binding fragment may be chemically or recombinantly synthesized (not naturally occurring).

### 4-1BB Targeting Moiety

The anti-HER2/anti-4-1BB bispecific antibody may comprise an anti-4-1BB antibody or an antigen-binding fragment thereof, as a 4-1BB targeting moiety.

The term “4-1BB”, which is also called as CD137 or TNFRSF9 (TNF Receptor Superfamily Member 9), is a member of TNF-receptor superfamily (TNFRSF) and is a co-stimulatory molecule which is expressed following the activation of immune cells, both innate and adaptive immune cells. 4-1BB plays important role in modulate the activity of various immune cells. As used herein, 4-1BB may be originated from a mammal, for example, Homo sapiens (human) (NCBI Accession No. NP_001552.2). For example, the human 4-1BB protein (NP_001552.2) may be represented by the amino acid sequence (SEQ ID NO:89), as follows:
1 mgnscyniva tlllvlnfer trslqdpcsn cpagtfcdnn rnqicspcpp nsfssaggqr
61 tcdicrackg vfrtrkecss tsnaecdctp gfhclgages mceqdckqgq eltkkgckdc
121 cfgtfndqkr gicrpwtncs ldgksvlvng tkerdvvcgp spadlspgas svtppapare
181 pghspqiisf flaltstall fllffltlrf svvkrgrkkl lyifkqpfmr pvqttqeedg
241 cscrfpeeee ggcel

In an embodiment, the anti-4-1BB antibody or an antigen-binding fragment thereof may comprise:
a CDR (complementarity determining region)-H1 (H-CDR1) comprising an amino acid sequence of SEQ ID NO:1, 2, or 3;
an H-CDR2 comprising an amino acid sequence of SEQ ID NO:4, 5, or 6;
an H-CDR3 comprising an amino acid sequence of SEQ ID NO:7, 8, 9, 10, or 11;
an L-CDR1 comprising an amino acid sequence of SEQ ID NO:12 or 13;
an L-CDR2 comprising an amino acid sequence of SEQ ID NO:14 or 15; and
an L-CDR3 comprising an amino acid sequence of SEQ ID NO:16 or 17.

The amino acid sequences of the CDRs of the anti-4-1BB antibody or an antigen-binding fragment are illustrated in Table 1:

TABLE 1

| SEQ | | SEQ | | SEQ | |
|---|---|---|---|---|---|
| ID | | ID | | ID | |
| NO | H-CDR1 | NO | H-CDR2 | NO | H-CDR3 |
| 1 | SYDMS | 4 | WISYSGGSIY | 7 | DGQRNSMREFDY |
| | | | YADSVKG | 8 | DAQRNSMREFDY |
| | | | | 9 | DAQRQSMREFDY |
| 2 | GYDMS | 5 | VIYPDDGNTY | 10 | HGGQKPTTKSSS |
| | | | YADSVKG | | AYGMDG |
| 3 | SYWMH | 6 | EINPGNGHTN | 11 | SFTTARAFAY |
| | | | YNEKFKS | | |
| SEQ | | SEQ | | SEQ | |
| ID | | ID | | ID | |
| NO | L-CDR1 | NO | L-CDR2 | NO | L-CDR3 |
| 12 | SGSSSNIGNNY | 14 | ADSHRPS | 16 | ATWDYSLSGYV |
| | VT | | | | |
| 13 | RASQTISDYLH | 15 | YASQSIS | 17 | QDGHSFPPT |

For example, the anti-4-1BB antibody or an antigen-binding fragment thereof may comprise:
an H-CDR1 comprising an amino acid sequence of SEQ ID NO:1, an H-CDR2 comprising an amino acid sequence of SEQ ID NO:4, an H-CDR3 comprising an amino acid sequence of SEQ ID NO:7, an L-CDR1 comprising an amino acid sequence of SEQ ID NO:12, an L-CDR2 comprising an amino acid sequence of SEQ ID NO:14, and an L-CDR3 comprising an amino acid sequence of SEQ ID NO:16;
an H-CDR1 comprising an amino acid sequence of SEQ ID NO:1, an H-CDR2 comprising an amino acid sequence of SEQ ID NO:4, an H-CDR3 comprising an amino acid sequence of SEQ ID NO:8, an L-CDR1 comprising an amino acid sequence of SEQ ID NO:12, an L-CDR2 comprising an amino acid sequence of SEQ ID NO:14, and an L-CDR3 comprising an amino acid sequence of SEQ ID NO:16;
an H-CDR1 comprising an amino acid sequence of SEQ ID NO:1, an H-CDR2 comprising an amino acid sequence of SEQ ID NO:4, an H-CDR3 comprising an amino acid sequence of SEQ ID NO:9, an L-CDR1 comprising an amino acid sequence of SEQ ID NO:12, an L-CDR2 comprising an amino acid sequence of SEQ ID NO:14, and an L-CDR3 comprising an amino acid sequence of SEQ ID NO:16;
an H-CDR1 comprising an amino acid sequence of SEQ ID NO:1, an H-CDR2 comprising an amino acid sequence of SEQ ID NO:4, an H-CDR3 comprising an amino acid sequence of SEQ ID NO:7, an L-CDR1 comprising an amino acid sequence of SEQ ID NO:13, an L-CDR2 comprising an amino acid sequence of SEQ ID NO:15, and an L-CDR3 comprising an amino acid sequence of SEQ ID NO:17;
an H-CDR1 comprising an amino acid sequence of SEQ ID NO:1, an H-CDR2 comprising an amino acid sequence of SEQ ID NO:4, an H-CDR3 comprising an amino acid sequence of SEQ ID NO:8, an L-CDR1 comprising an amino acid sequence of SEQ ID NO:13, an L-CDR2 comprising an amino acid sequence of SEQ ID NO:15, and an L-CDR3 comprising an amino acid sequence of SEQ ID NO:17;
an H-CDR1 comprising an amino acid sequence of SEQ ID NO:1, an H-CDR2 comprising an amino acid sequence of SEQ ID NO:4, an H-CDR3 comprising an amino acid sequence of SEQ ID NO:9, an L-CDR1 comprising an amino acid sequence of SEQ ID NO:13, an L-CDR2 comprising an amino acid sequence of SEQ ID NO:15, and an L-CDR3 comprising an amino acid sequence of SEQ ID NO:17;
an H-CDR1 comprising an amino acid sequence of SEQ ID NO:2, an H-CDR2 comprising an amino acid sequence of SEQ ID NO:5, an H-CDR3 comprising an amino acid sequence of SEQ ID NO:10, an L-CDR1 comprising an amino acid sequence of SEQ ID NO:12, an L-CDR2 comprising an amino acid sequence of SEQ ID NO:14, and an L-CDR3 comprising an amino acid sequence of SEQ ID NO:16;
an H-CDR1 comprising an amino acid sequence of SEQ ID NO:2, an H-CDR2 comprising an amino acid sequence of SEQ ID NO:5, an H-CDR3 comprising an amino acid sequence of SEQ ID NO:10, an L-CDR1 comprising an amino acid sequence of SEQ ID NO:13, an L-CDR2 comprising an amino acid sequence of SEQ ID NO:15, and an L-CDR3 comprising an amino acid sequence of SEQ ID NO:17;
an H-CDR1 comprising an amino acid sequence of SEQ ID NO:3, an H-CDR2 comprising an amino acid sequence of SEQ ID NO:6, an H-CDR3 comprising an amino acid sequence of SEQ ID NO:11, an L-CDR1 comprising an amino acid sequence of SEQ ID NO:12, an L-CDR2 comprising an amino acid sequence of SEQ ID NO:14, and an L-CDR3 comprising an amino acid sequence of SEQ ID NO:16; or
an H-CDR1 comprising an amino acid sequence of SEQ ID NO:3, an H-CDR2 comprising an amino acid sequence of SEQ ID NO:6, an H-CDR3 comprising an amino acid sequence of SEQ ID NO:11, an L-CDR1 comprising an amino acid sequence of SEQ ID NO:13, an L-CDR2 comprising an amino acid sequence of SEQ ID NO:15, and an L-CDR3 comprising an amino acid sequence of SEQ ID NO:17.

In another embodiment, the anti-4-1BB antibody or antigen-binding fragment thereof may comprise a heavy chain variable region comprising an H-CDR1 comprising an amino acid sequence of SEQ ID NO:1, 2, or 3, an H-CDR2 comprising an amino acid sequence of SEQ ID NO:4, 5, or 6, and an H-CDR3 comprising an amino acid sequence of SEQ ID NO:7, 8, 9, 10, or 11; and a light chain variable region comprising an L-CDR1 comprising an amino acid sequence of SEQ ID NO:12, or 13, an L-CDR2 comprising an amino acid sequence of SEQ ID NO:14, or 15, and an L-CDR3 comprising an amino acid sequence of SEQ ID NO:16, or 17.

In another embodiment, the anti-4-1BB antibody or antigen-binding fragment thereof may comprise a heavy chain variable region comprising an amino acid sequence of SEQ ID NO:18, 19, 20, 21, 22, 23, 24, 25, 26, 27, 28, or 29; and a light chain variable region comprising or consisting essentially of an amino acid sequence of SEQ ID NO:30, 31, 32, 33, 34, or 88.

The amino acid sequences of the variable regions of the anti-4-1BB antibody or an antigen-binding fragment are illustrated in Table 2:

TABLE 2

| SEQ | |
|---|---|
| ID | |
| NO | Heavy chain variable region targeting 4-1BB |
| 18 | EVQLLESGGGLVQPGGSLRLSCAASGFTFSSYDMSWVRQAPGKGLE |
| | WVSWISYSGGSIYYADSVKGRFTISRDNSKNTLYLQMNSLRAEDTA |
| | VYYCARDGQRNSMREFDYWGQGTLVTVSS |
| 19 | EVQLLESGGGLVQPGGSLRLSCAASGFTFSSYDMSWVRQAPGKGLE |
| | WVSWISYSGGSIYYADSVKGRFTISRDNSKNTLYLQMNSLRAEDTA |
| | VYYCARDAQRNSMREFDYWGQGTLVTVSS |
| 20 | EVQLLESGGGLVQPGGSLRLSCAASGFTFSSYDMSWVRQAPGKGLE |
| | WVSWISYSGGSIYYADSVKGRFTISRDNSKNTLYLQMNSLRAEDTA |
| | VYYCARDAQRQSMREFDYWGQGTLVTVSS |
| 21 | EVQLLESGGGLVQPGGSLRLSCAASGFTFSGYDMSWVRQAPGKGLE |
| | WVSVIYPDDGNTYYADSVKGRFTISRDNSKNTLYLQMNSLRAEDAA |
| | VYYCAKHGGQKPTTKSSSAYGMDGWGQGTLVTVSS |
| 22 | EVQLLESGGGLVQPGGSLRLSCAASGFTFSGYDMSWVRQAPGKGLE |
| | WVSVIYPDDGNTYYADSVKGRFTISRDNSKNTLYLQMNSLRAEDTA |
| | VYYCAKHGGQKPTTKSSSAYGMDGWGQGTLVTVSS |
| 23 | QVQLQQSGAEVIKPGASVKLSCKASGYTFSSYWMHWVRQAPGQGLE |
| | WIGEINPGNGHTNYNEKFKSRATLTGDTSTSTVYMELSSLRSEDTA |
| | VYYCARSFTTARAFAYWGQGTLVTVSS |
| 24 | EVQLLESGGGLVQPGGSLRLSCAASGFTFSSYDMSWVRQAPGKCLE |
| | WVSWISYSGGSIYYADSVKGRFTISRDNSKNTLYLQMNSLRAEDTA |
| | VYYCARDGQRNSMREFDYWGQGTLVTVSS |
| 25 | EVQLLESGGGLVQPGGSLRLSCAASGFTFSSYDMSWVRQAPGKCLE |
| | WVSWISYSGGSIYYADSVKGRFTISRDNSKNTLYLQMNSLRAEDTA |
| | VYYCARDAQRNSMREFDYWGQGTLVTVSS |
| 26 | EVQLLESGGGLVQPGGSLRLSCAASGFTFSSYDMSWVRQAPGKCLE |
| | WVSWISYSGGSIYYADSVKGRFTISRDNSKNTLYLQMNSLRAEDTA |
| | VYYCARDAQRQSMREFDYWGQGTLVTVSS |
| 27 | EVQLLESGGGLVQPGGSLRLSCAASGFTFSGYDMSWVRQAPGKCLE |
| | WVSVIYPDDGNTYYADSVKGRFTISRDNSKNTLYLQMNSLRAEDAA |
| | VYYCAKHGGQKPTTKSSSAYGMDGWGQGTLVTVSS |
| 28 | EVQLLESGGGLVQPGGSLRLSCAASGFTFSGYDMSWVRQAPGKCLE |
| | WVSVIYPDDGNTYYADSVKGRFTISRDNSKNTLYLQMNSLRAEDTA |
| | VYYCAKHGGQKPTTKSSSAYGMDGWGQGTLVTVSS |
| 29 | QVQLQQSGAEVIKPGASVKLSCKASGYTFSSYWMHWVRQAPGQCLE |
| | WIGEINPGNGHTNYNEKFKSRATLTGDTSTSTVYMELSSLRSEDTA |
| | VYYCARSFTTARAFAYWGQGTLVTVSS |
| SEQ | |
| ID | |
| NO | Light chain variable region targeting 4-1BB |
| 30 | QSVLTQPPSASGTPGRRVTISCSGSSSNIGNNYVTWYQQLPGTAPK |
| | LLIYADSHRPSGVPDRFSGSKSGTSASLAISGLRSEDEADYYCATW |
| | DYSLSGYVFGGGTKLTVL |
| 31 | QSVLTQPPSASGTPGQRVTISCSGSSSNIGNNYVTWYQQLPGTAPK |
| | LLIYADSHRPSGVPDRFSGSKSGTSASLAISGLRSEDEADYYCATW |
| | DYSLSGYVFGGGTKLTVL |
| 32 | DIVMTQSPAFLSVTPGEKVTITCRASQTISDYLHWYQQKPDQAPKL |
| | LIKYASQSISGIPSRFSGSGSGTDFTFTISSLEAEDAATYYCQDGH |
| | SFPPTFGQGTKLEIKR |
| 33 | QSVLTQPPSASGTPGRRVTISCSGSSSNIGNNYVTWYQQLPGTAPK |
| | LLIYADSHRPSGVPDRFSGSKSGTSASLAISGLRSEDEADYYCATW |
| | DYSLSGYVFGCGTKLTVL |
| 34 | QSVLTQPPSASGTPGQRVTISCSGSSSNIGNNYVTWYQQLPGTAPK |
| | LLIYADSHRPSGVPDRFSGSKSGTSASLAISGLRSEDEADYYCATW |
| | DYSLSGYVFGCGTKLTVL |
| 88 | DIVMTQSPAFLSVTPGEKVTITCRASQTISDYLHWYQQKPDQAPKL |
| | LIKYASQSISGIPSRFSGSGSGTDFTFTISSLEAEDAATYYCQDGH |
| | SFPPTFGCGTKLEIKR |

For example, the anti-4-1BB antibody or an antigen-binding fragment thereof may comprise:
a heavy chain variable region comprising an comprising an amino acid sequence of SEQ ID NO:18, 19, 20, 21, 22, 23, 24, 25, 26, 27, 28, or 29, and a light chain variable region comprising an amino acid sequence of SEQ ID NO:30;
a heavy chain variable region comprising an comprising an amino acid sequence of SEQ ID NO:18, 19, 20, 21, 22, 23, 24, 25, 26, 27, 28, or 29, and a light chain variable region comprising an amino acid sequence of SEQ ID NO:31;
a heavy chain variable region comprising an comprising an amino acid sequence of SEQ ID NO:18, 19, 20, 21, 22, 23, 24, 25, 26, 27, 28, or 29, and a light chain variable region comprising an amino acid sequence of SEQ ID NO:32;
a heavy chain variable region comprising an comprising an amino acid sequence of SEQ ID NO:18, 19, 20, 21, 22, 23, 24, 25, 26, 27, 28, or 29, and a light chain variable region comprising an amino acid sequence of SEQ ID NO:33;
a heavy chain variable region comprising an comprising an amino acid sequence of SEQ ID NO:18, 19, 20, 21, 22, 23, 24, 25, 26, 27, 28, or 29, and a light chain variable region comprising an amino acid sequence of SEQ ID NO:34; or
a heavy chain variable region comprising an comprising an amino acid sequence of SEQ ID NO:18, 19, 20, 21, 22, 23, 24, 25, 26, 27, 28, or 29, and a light chain variable region comprising an amino acid sequence of SEQ ID NO:88.

The amino acid sequences of frameworks of the variable regions of the anti-4-1BB antibody or an antigen-binding fragment are illustrated in Table 3:

TABLE 3

| SEQ | | SEQ | | SEQ | | SEQ | |
|---|---|---|---|---|---|---|---|
| ID | | ID | | ID | | ID | |
| NO | H-FR1 | NO | H-FR2 | NO | H-FR3 | NO | H-FR4 |
| 35 | EVQLLESGG | 37 | WVRQAP | 41 | RFTISRDNS | 45 | WGQGT |
| | GLVQPGGSL | | GKGLEWV | | KNTLYLQMN | | LVTVSS |
| | RLSCAASGF | | S | | SLRAEDTAV | | |
| | TFS | | | | YYCAR | | |
| 35 | EVQLLESGG | 37 | WVRQAP | 42 | RFTISRDNS | 45 | WGQGT |
| | GLVQPGGSL | | GKGLEWV | | KNTLYLQMN | | LVTVSS |
| | RLSCAASGF | | S | | SLRAEDAAV | | |
| | TFS | | | | YYCAK | | |
| 35 | EVQLLESGG | 37 | WVRQAP | 43 | RFTISRDNS | 45 | WGQGT |
| | GLVQPGGSL | | GKGLEWV | | KNTLYLQMN | | LVTVSS |
| | RLSCAASGF | | S | | SLRAEDTAV | | |
| | TFS | | | | YYCAK | | |
| 36 | QVQLQQSG | 38 | WVRQAP | 44 | RATLTGDTS | 45 | WGQGT |
| | AEVIKPGAS | | GQGLEWI | | TSTVYMELS | | LVTVSS |
| | VKLSCKASG | | G | | SLRSEDTAV | | |
| | YTFS | | | | YYCAR | | |
| 35 | EVQLLESGG | 39 | WVRQAP | 41 | RFTISRDNS | 45 | WGQGT |
| | GLVQPGGSL | | GKCLEWV | | KNTLYLQMN | | LVTVSS |
| | RLSCAASGF | | S | | SLRAEDTAV | | |
| | TFS | | | | YYCAR | | |
| 35 | EVQLLESGG | 39 | WVRQAP | 42 | RFTISRDNS | 45 | WGQGT |
| | GLVQPGGSL | | GKCLEWV | | KNTLYLQMN | | LVTVSS |
| | RLSCAASGF | | S | | SLRAEDAAV | | |
| | TFS | | | | YYCAK | | |
| 35 | EVQLLESGG | 39 | WVRQAP | 43 | RFTISRDNS | 45 | WGQGT |
| | GLVQPGGSL | | GKCLEWV | | KNTLYLQMN | | LVTVSS |
| | RLSCAASGF | | S | | SLRAEDTAV | | |
| | TFS | | | | YYCAK | | |
| 36 | QVQLQQSG | 40 | WVRQAP | 44 | RATLTGDTS | 45 | WGQGT |
| | AEVIKPGAS | | GQCLEWI | | TSTVYMELS | | LVTVSS |
| | VKLSCKASG | | G | | SLRSEDTAV | | |
| | YTFS | | | | YYCAR | | |
| SEQ | | SEQ | | SEQ | | SEQ | |
| ID | | ID | | ID | | ID | |
| NO | L-FR1 | NO | L-FR2 | NO | L-FR3 | NO | L-FR4 |
| 46 | QSVLTQPPS | 49 | WYQQLP | 51 | GVPDRFSG | 53 | FGGGT |
| | ASGTPGRR | | GTAPKLLI | | SKSGTSASL | | KLTVL |
| | VTISC | | Y | | AISGLRSED | | |
| | | | | | EADYYC | | |
| 47 | QSVLTQPPS | 49 | WYQQLP | 51 | GVPDRFSG | 53 | FGGGT |
| | ASGTPGQR | | GTAPKLLI | | SKSGTSASL | | KLTVL |
| | VTISC | | Y | | AISGLRSED | | |
| | | | | | EADYYC | | |
| 48 | DIVMTQSPA | 50 | WYQQKP | 52 | GIPSRFSGS | 54 | FGQGT |
| | FLSVTPGEK | | DQAPKLLI | | GSGTDFTFT | | KLEIKR |
| | VTITC | | K | | ISSLEAEDAA | | |
| | | | | | TYYC | | |
| 46 | QSVLTQPPS | 49 | WYQQLP | 51 | GVPDRFSG | 55 | FGCGT |
| | ASGTPGRR | | GTAPKLLI | | SKSGTSASL | | KLTVL |
| | VTISC | | Y | | AISGLRSED | | |
| | | | | | EADYYC | | |
| 47 | QSVLTQPPS | 49 | WYQQLP | 51 | GVPDRFSG | 55 | FGCGT |
| | ASGTPGQR | | GTAPKLLI | | SKSGTSASL | | KLTVL |
| | VTISC | | Y | | AISGLRSED | | |
| | | | | | EADYYC | | |

In another embodiment, the anti-4-1BB antibody or antigen-binding fragment thereof may comprise a heavy chain comprising or consisting essentially of an amino acid sequence of SEQ ID NO:56, 57, 58, 59, 60, or 61; and a light chain comprising or consisting essentially of an amino acid sequence of SEQ ID NO:62, 63, or 64.

For example, the anti-4-1BB antibody or an antigen-binding fragment thereof may comprise:
a heavy chain comprising or consisting essentially of an amino acid sequence of SEQ ID NO:56, 57, 58, 59, 60, or 61; and a light chain comprising or consisting essentially of an amino acid sequence of SEQ ID NO:62;
a heavy chain comprising or consisting essentially of an amino acid sequence of SEQ ID NO:56, 57, 58, 59, 60, or 61; and a light chain comprising or consisting essentially of an amino acid sequence of SEQ ID NO:63; or
a heavy chain comprising or consisting essentially of an amino acid sequence of SEQ ID NO:56, 57, 58, 59, 60, or 61; and a light chain comprising or consisting essentially of an amino acid sequence of SEQ ID NO:64.

In another embodiment, the anti-4-1BB antibody or antigen-binding fragment thereof may be a scFv (single chain variable fragment), comprising:
a heavy chain variable region comprising an H-CDR1 comprising an amino acid sequence of SEQ ID NO:1, 2, or 3, an H-CDR2 comprising an amino acid sequence of SEQ ID NO:4, 5, or 6, and an H-CDR3 comprising an amino acid sequence of SEQ ID NO:7, 8, 9, 10, or 11; and
a light chain variable region comprising an L-CDR1 comprising an amino acid sequence of SEQ ID NO:12, or 13, an L-CDR2 comprising an amino acid sequence of SEQ ID NO:14, or 15, and an L-CDR3 comprising an amino acid sequence of SEQ ID NO:16, or 17,
wherein the heavy chain variable region and the light chain variable region may be linked to each other in any order directly (i.e., without a linker) or via a peptide linker.

For example, the anti-4-1BB scFv may comprise:
a heavy chain variable region comprising an amino acid sequence of SEQ ID NO:18, 19, 20, 21, 22, 23, 24, 25, 26, 27, 28, or 29; and
a light chain variable region comprising or consisting essentially of an amino acid sequence of SEQ ID NO:30, 31, 32, 33, 34, or 88,
wherein the heavy chain variable region and the light chain variable region may be linked to each other in any order directly or via a peptide linker.

For example, the anti-4-1BB scFv may comprise:
a heavy chain variable region comprising or consisting essentially of an amino acid sequence of SEQ ID NO:24, 25, 26, 27, 28, or 29; and a light chain variable region comprising or consisting essentially of an amino acid sequence of SEQ ID NO:33;
a heavy chain variable region comprising or consisting essentially of an amino acid sequence of SEQ ID NO:24, 25, 26, 27, 28, or 29; and a light chain variable region comprising or consisting essentially of an amino acid sequence of SEQ ID NO:34; or
a heavy chain variable region comprising an comprising an amino acid sequence of SEQ ID NO:24, 25, 26, 27, 28, or 29, and a light chain variable region comprising an amino acid sequence of SEQ ID NO:88,
wherein the heavy chain variable region and the light chain variable region may be linked to each other in any order directly or via a peptide linker.

In the present disclosure, the anti-4-1BB scFv comprise a heavy chain variable region and a light chain variable region, in any order. For example, the anti-4-1BB scFv may comprise a light chain variable region and a heavy chain variable region, in a direction from N-terminus to C-terminus. Alternatively, the anti-4-1BB scFv may comprise a heavy chain variable region and a light chain variable region, in a direction from N-terminus to C-terminus.

### HER2 Targeting Moiety

The anti-HER2/anti-4-1BB bispecific antibody may comprise an anti-HER2 antibody or an antigen-binding fragment thereof as a HER2 targeting moiety.

The “HER2 (human epidermal growth factor receptor 2)” is encoded by ERBB2 gene, and is a member of the epidermal growth factor receptor (EGFR/ErbB). HER2 has been known to play an essential role in regulating cell proliferation and differentiation. Particularly, when bound to extracellular growth factors, it has a strong tendency of being assembled into homo- and/or heterodimers along with other HER receptors, which results in the activation of several forms of signal transduction pathway and induces apoptosis, survival, or cell proliferation. For instance, the HER2 protein may be polypeptides deposited under GenBank Accession Number NP_004439.2, NP_001005862.1, etc. which are encoded by the nucleotide sequences (mRNA) deposited under GenBank Accession Number NM 004448.4, NM_001005862.3, etc., respectively.

In one embodiment, the anti-HER2 antibody may be selected from the group consisting of Trastuzumab, Pertuzumab, and Trastuzumab emtansine (T-DM1).

The antigen binding region of the anti-HER2 antibody recognizing HER2 as an antigen may be scFv, (scFv)₂, Fab, Fab′ or F(ab′)₂ of an anti-HER2 antibody selected from the group consisting of Trastuzumab, Pertuzumab, and Trastuzumab emtansine (T-DM1).

The anti-HER2 antibody or an antigen-binding fragment thereof may be an anti-HER2 antibody or an antigen-binding fragment thereof comprising 6 CDRs of Trastuzumab, Pertuzumab, or Trastuzumab emtansine (T-DM1).

In an embodiment, the anti-HER2 antibody or antigen-binding fragment thereof may be trastuzumab or an antigen-binding fragment thereof, or a variant thereof.

For example, the anti-HER2 antibody or antigen-binding fragment thereof may comprise:
an H-CDR1 comprising an amino acid sequence of SEQ ID NO:65;
an H-CDR2 comprising an amino acid sequence of SEQ ID NO:66;
an H-CDR3 comprising an amino acid sequence of SEQ ID NO:67;
an L-CDR1 comprising an amino acid sequence of SEQ ID NO:68;
an L-CDR2 comprising an amino acid sequence of SEQ ID NO:69; and
an L-CDR3 comprising an amino acid sequence of SEQ ID NO:70.

The amino acid sequences of the CDRs of the anti-HER2 antibody or an antigen-binding fragment are illustrated in Table 4:

TABLE 4

| SEQ | | SEQ | | SEQ | |
|---|---|---|---|---|---|
| ID NO | VH_CDR1 | ID NO | VH_CDR2 | ID NO | VH_CDR3 |
| 65 | DTYIH | 66 | RIYPTNGYTRYA | 67 | WGGDGFYAM |
| | | | DSVKG | | DY |
| SEQ | | SEQ | | SEQ | |
| ID NO | L-CDR1 | ID NO | L-CDR2 | ID NO | L-CDR3 |
| 68 | RASQDVNT | 69 | SASFLYS | 70 | QQHYTTPPT |
| | AVA | | | | |

In another embodiment, the anti-HER2 antibody or antigen-binding fragment thereof may comprise a heavy chain variable region comprising an H-CDR1 comprising an amino acid sequence of SEQ ID NO:65, an H-CDR2 comprising an amino acid sequence of SEQ ID NO:66, and an H-CDR3 comprising an amino acid sequence of SEQ ID NO:67; and a light chain variable region comprising an L-CDR1 comprising an amino acid sequence of SEQ ID NO:68, an L-CDR2 comprising an amino acid sequence of SEQ ID NO:69, and an L-CDR3 comprising an amino acid sequence of SEQ ID NO:70.

In another embodiment, the anti-HER2 antibody or antigen-binding fragment thereof may comprise a heavy chain variable region comprising or consisting essentially of an amino acid sequence of SEQ ID NO:71, and a light chain variable region comprising or consisting essentially of an amino acid sequence of SEQ ID NO:72.

The amino acid sequences of the variable regions of the anti-HER2 antibody or an antigen-binding fragment are illustrated in Table 5:

TABLE 5

| SEQ | |
|---|---|
| ID NO | Heavy chain variable region targeting HER2 |
| 71 | EVQLVESGGGLVQPGGSLRLSCAASGFNIKDTYIHWVRQAPGKGL |
| | EWVARIYPTNGYTRYADSVKGRFTISADTSKNTAYLQMNSLRAED |
| | TAVYYCSRWGGDGFYAMDYWGQGTLVTVS |
| SEQ | |
| ID NO | Light chain variable region targeting HER2 |
| 72 | DIQMTQSPSSLSASVGDRVTITCRASQDVNTAVAWYQQKPGKAP |
| | KLUYSASFLYSGVPSRFSGSRSGTDFILTISSLOPEDFATYYCQQ |
| | HYTTPPTFGQGTKVEIKR |

In another embodiment, the anti-HER2 antibody or antigen-binding fragment thereof may comprise a heavy chain comprising or consisting essentially of an amino acid sequence of SEQ ID NO:73, or 74; and a light chain comprising or consisting essentially of an amino acid sequence of SEQ ID NO:75.

In another embodiment, the anti-HER2 antibody or antigen-binding fragment thereof may be a scFv (single chain variable fragment), comprising:
a heavy chain variable region comprising an H-CDR1 comprising an amino acid sequence of SEQ ID NO:65, an H-CDR2 comprising an amino acid sequence of SEQ ID NO:66, and an H-CDR3 comprising an amino acid sequence of SEQ ID NO:67; and
a light chain variable region comprising an L-CDR1 comprising an amino acid sequence of SEQ ID NO:68, an L-CDR2 comprising an amino acid sequence of SEQ ID NO:69, and an L-CDR3 comprising an amino acid sequence of SEQ ID NO:70,
wherein the heavy chain variable region and the light chain variable region may be linked to each other in any order directly (i.e., without a linker) or via a peptide linker.

In another embodiment, the anti-HER2 antibody or antigen-binding fragment thereof may be a scFv (single chain variable fragment), comprising:
a heavy chain variable region comprising or consisting essentially of an amino acid sequence of SEQ ID NO:71; and
a light chain variable region comprising or consisting essentially of an amino acid sequence of SEQ ID NO:72,
wherein the heavy chain variable region and the light chain variable region may be linked to each other in any order directly or via a peptide linker.

In the present disclosure, the anti-HER2 scFv comprise a heavy chain variable region and a light chain variable region, in any order. For example, the anti-HER2 scFv may comprise a light chain variable region and a heavy chain variable region, in a direction from N-terminus to C-terminus. Alternatively, the anti-HER2 scFv may comprise a heavy chain variable region and a light chain variable region, in a direction from N-terminus to C-terminus.

### Bispecific Antibody

The present disclosure provides an anti-HER2/anti-4-1BB bispecific antibody comprising:
(1) an anti-HER2 antibody or an antigen-binding fragment thereof, as a HER2 targeting moiety, which is capable of specifically recognizing and/or binding to HER2 protein, and
(2) an anti-4-1BB antibody or an antigen-binding fragment thereof, as a 4-1BB targeting moiety, which is capable of specifically recognizing and/or binding to 4-1BB protein.

The anti-HER2/anti-4-1BB bispecific antibody may activate 4-1BB signaling only when crosslinked by HER2-expressing tumor cells. In addition, the anti-4-1BB antibody or an antigen-binding fragment thereof contained in the bispecific antibody may be characterized by localizing and/or activating only in tumor microenvironment (TME), and/or considerably reducing liver toxicities compared to pre-existing anti-4-1BB antibodies, with maintaining the efficacies of immune response enhancement and/or tumor treatment.

In an embodiment, the bispecific antibody may comprise a full-length anti-HER2 antibody and an antigen-binding fragment (e.g., scFv) of an anti-4-1BB antibody, wherein the antigen-binding fragment of an anti-4-1BB antibody may be linked to N-terminus, C-terminus, or both thereof of a full-length anti-HER2 antibody, directly or via a peptide linker. In another embodiment, the bispecific antibody may comprise a full-length anti-4-1BB antibody and an antigen-binding fragment (e.g., scFv) of an anti-HER2 antibody, wherein the antigen-binding fragment of an anti-HER2 antibody may be linked to N-terminus, C-terminus, or both thereof of a full-length anti-4-1BB antibody, directly or via a peptide linker.

In an embodiment, the scFv contained in the bispecific antibody may comprise a heavy chain variable region and a light chain variable region in any order. For example, the scFv contained in the bispecific antibody may comprise a light chain variable region and a heavy chain variable region, in a direction from N-terminus to C-terminus, and optionally a peptide linker therebetween, or alternatively, the scFv contained in the bispecific antibody may comprise a heavy chain variable region and a light chain variable region, in a direction from N-terminus to C-terminus, and optionally a peptide linker therebetween.

When the bispecific antibody comprises a full-length anti-HER2 antibody and an anti-4-1BB scFv, the bispecific antibody may comprise:
(i) a first polypeptide comprising, in a direction from N-terminus to C-terminus:
a heavy chain of an anti-HER2 antibody,
optionally, a peptide linker (a first peptide linker), and
an anti-4-1BB scFv; and
(ii) a second polypeptide comprising a light chain of the anti-HER2 antibody,
wherein the anti-4-1BB scFv may comprise, in a direction from N-terminus to C-terminus:
a light chain variable region of an anti-4-1BB antibody,
optionally, a peptide linker (a second peptide linker), and
a heavy chain variable region of the anti-4-1BB antibody.

Alternatively, the bispecific antibody may comprise:
(i) a first polypeptide comprising, in a direction from N-terminus to C-terminus:
an anti-4-1BB scFv,
optionally, a peptide linker (a first peptide linker), and
a heavy chain of an anti-HER2 antibody; and
(ii) a second polypeptide comprising a light chain of the anti-HER2 antibody,
wherein the anti-4-1BB scFv may comprise, in a direction from N-terminus to C-terminus:
a light chain variable region of an anti-4-1BB antibody,
optionally, a peptide linker (a second peptide linker), and
a heavy chain variable region of the anti-4-1BB antibody.

Alternatively, the bispecific antibody may comprise:
(i) a first polypeptide comprising, in a direction from N-terminus to C-terminus:
a heavy chain of an anti-HER2 antibody,
optionally, a peptide linker (a first peptide linker), and
an anti-4-1BB scFv; and
(ii) a second polypeptide comprising a light chain of the anti-HER2 antibody,
wherein the anti-4-1BB scFv may comprise, in a direction from N-terminus to C-terminus:
a heavy chain variable region of the anti-4-1BB antibody,
optionally, a peptide linker (a second peptide linker), and
a light chain variable region of an anti-4-1BB antibody.

Alternatively, the bispecific antibody may comprise:
(i) a first polypeptide comprising, in a direction from N-terminus to C-terminus:
an anti-4-1BB scFv,
optionally, a peptide linker (a first peptide linker), and
a heavy chain of an anti-HER2 antibody; and
(ii) a second polypeptide comprising a light chain of the anti-HER2 antibody,
wherein the anti-4-1BB scFv may comprise, in a direction from N-terminus to C-terminus:
a heavy chain variable region of the anti-4-1BB antibody,
optionally, a peptide linker (a second peptide linker), and
a light chain variable region of an anti-4-1BB antibody.

When the bispecific antibody comprises a full-length anti-4-1BB antibody and an anti-HER2 scFv, the bispecific antibody may comprise:
(i) a first polypeptide comprising, in a direction from N-terminus to C-terminus:
a heavy chain of an anti-4-1BB antibody,
optionally, a peptide linker (a first peptide linker), and
an anti-HER2 scFv; and
(ii) a second polypeptide comprising a light chain of the anti-4-1BB antibody,
wherein the anti-HER2 scFv may comprise, in a direction from N-terminus to C-terminus:
a light chain variable region of an anti-HER2 antibody,
optionally, a peptide linker (a second peptide linker), and
a heavy chain variable region of the anti-HER2 antibody.

Alternatively, the bispecific antibody may comprise:
(i) a first polypeptide comprising, in a direction from N-terminus to C-terminus:
an anti-HER2 scFv,
optionally, a peptide linker (a first peptide linker), and
a heavy chain of an anti-4-1BB antibody; and
(ii) a second polypeptide comprising a light chain of the anti-4-1BB antibody,
wherein the anti-HER2 scFv may comprise, in a direction from N-terminus to C-terminus:
a light chain variable region of an anti-HER2 antibody,
optionally, a peptide linker (a second peptide linker), and
a heavy chain variable region of the anti-HER2 antibody.

Alternatively, the bispecific antibody may comprise:
(i) a first polypeptide comprising, in a direction from N-terminus to C-terminus:
a heavy chain of an a anti-4-1BB antibody,
optionally, a peptide linker (a first peptide linker), and
an anti-HER2 scFv; and
(ii) a second polypeptide comprising a light chain of the anti-4-1BB antibody,
wherein the anti-HER2 scFv may comprise, in a direction from N-terminus to C-terminus:
a heavy chain variable region of the anti-HER2 antibody,
optionally, a peptide linker (a second peptide linker), and
a light chain variable region of an anti-HER2 antibody.

Alternatively, the bispecific antibody may comprise:
(i) a first polypeptide comprising, in a direction from N-terminus to C-terminus:
an anti-HER2 scFv,
optionally, a peptide linker (a first peptide linker), and
a heavy chain of an anti-4-1BB antibody; and
(ii) a second polypeptide comprising a light chain of the anti-4-1BB antibody,
wherein the anti-HER2 scFv may comprise, in a direction from N-terminus to C-terminus:
a heavy chain variable region of the anti-HER2 antibody,
optionally, a peptide linker (a second peptide linker), and
a light chain variable region of an anti-HER2 antibody.

The first peptide linker and the second peptide linker may be, independently, present or absent in the bispecific antibody, and the same with or different from each other.

In another embodiment, both of the HER2 targeting moiety and the 4-1BB targeting moiety contained in the bispecific antibody may be a full-length antibody or an antigen-binding fragment comprising heavy chain CDRs, light chain CDRs, or a combination thereof, which are linked to each other directly or via a peptide linker.

Given that each of antibodies can bind to both of 4-1BB (such as, human 4-1BB) and HER2 (such as, human HER2), the CDR sequences, or V_{H} (heavy chain variable region) and V_{L} (light chain variable region) sequences as disclosed herein can be “mixed and matched” to create other anti-HER2/anti-4-1BB binding bispecific molecules.

### Peptide Linker

For high purity of the antibody, the bispecific antibody may comprise a peptide linker between a heavy chain and scFv in a first polypeptide (a first peptide linker), and/or between heavy and light variable regions in scFv (a second peptide linker).

As used herein, the term “peptide linker” may refer to an oligopeptide including 1 to 100 amino acids, particularly 2 to 50 amino acids, each of which may be any kind of amino acids without any restrictions. Any conventional peptide linker may be used with or without an appropriate modification to comply with specific purposes. In a specific embodiment, the peptide linker may comprise, for example, Gly, Asn and/or Ser residues, and/or comprise neutral amino acids such as Thr and/or Ala. The amino acid sequences suitable for the peptide linker may be known in the relevant art. The length of the peptide linker can be properly determined within such a limit that the functions of the polypeptide and/or scFv will not be affected. For instance, the peptide linker may be formed by including a total of about 1 to about 100 amino acids, about 2 to about 50 amino acids, or about 5 to about 25 (e.g., 1, 2, 3, 4, 5, 6, 7, 8, 9, 10, 11, 12, 13, 14, 15, 16, 17, 18, 19, 20, 21, 22, 23, 24, or 25) amino acids, each of which is independently selected from the group consisting of Gly, Asn, Ser, Thr, and Ala. In one embodiment, the peptide linker may be represented as (GₘSₗ)ₙ (m, l, and n are the number of “G”, “S”, and “(GₘSₗ)”, respectively, and independently selected from integers of about 1 to about 10, particularly, 1, 2, 3, 4, 5, 6, 7, 8, 9, or 10). In one embodiment, the peptide linker can be amino acids of (GGGGS)2, (GGGGS)3, (GGGGS)4, or (GS)9, but not be limited thereto.

### Medical Use

Provided is a medical use of the bispecific antibody for enhancing immune response, and/or treating and/or preventing a cancer.

More specifically, an embodiment provides a pharmaceutical composition comprising the bispecific antibody as an active ingredient. The pharmaceutical composition may further comprise a pharmaceutically acceptable carrier. The pharmaceutical composition may be used for enhancing immune response, and/or for treating and/or preventing a cancer.

Another embodiment provides a pharmaceutical composition for treating and/or preventing a cancer, the composition comprising the bispecific antibody as an active ingredient.

Another embodiment provides a method of treating and/or preventing a cancer in a subject in need thereof, comprising administering to the subject a pharmaceutically effective amount of the bispecific antibody or the pharmaceutical composition. The method may further step of identifying the subject in need of treating and/or preventing a cancer, prior to the administering step.

Another embodiment provides a use of the bispecific antibody or the pharmaceutical composition in treating and/or preventing a cancer. Another embodiment provides a use of the bispecific antibody in preparing a medicament for treating and/or preventing a cancer.

In some embodiment, the cancer may be characterized by HER2 expression or HER2 overexpression (compared to normal).

Another embodiment provides a pharmaceutical composition for enhancing immune response, the composition comprising the bispecific antibody as an active ingredient.

Another embodiment provides a method of enhancing immune response in a subject in need thereof, comprising administering to the subject a pharmaceutically effective amount of the bispecific antibody or the pharmaceutical composition to the subject. The method may further comprise a step of identifying the subject in need of enhancing immune response, prior to the administering step.

Another embodiment provides a use of the bispecific antibody or the pharmaceutical composition in enhancing immune response. Another embodiment provides a use of the bispecific antibody in preparing a medicament for enhancing immune response.

In some embodiment, the bispecific antibody or the pharmaceutical composition may enhance immune response with the proviso of the presence of HER2. For example, in the method of enhancing immune response, the subject may have a HER2-expressing or HER2-overexpressing cell (e.g., a HER2-expressing or HER2-overexpressing cancer cell).

The cancer to be prevented and/or treated by the bispecific antibody or the pharmaceutical composition may be associated with 4-1BB and/or HER2, especially, HER2-expressed or HER2-overexpressed cancer. The cancer may be selected from solid cancers and blood cancers. The cancer may be, but not limited to, one or more selected from the group consisting of breast cancer, colon cancer, gastric cancer, lung cancer (e.g., squamous cell carcinoma of the lung, small-cell lung cancer, non-small-cell lung cancer, adenocarcinoma of the lung), peritoneal carcinoma, skin cancer, squamous cell carcinoma, melanoma in the skin or eyeball, rectal cancer, cancer near the anus, esophagus cancer, small intestinal tumor, endocrine gland cancer, parathyroid cancer, adrenal cancer, soft-tissue sarcoma, urethral cancer, chronic or acute leukemia, lymphocytic lymphoma, hepatoma, gastrointestinal cancer, pancreatic cancer, glioblastoma, cervical cancer, ovarian cancer, liver cancer, bladder cancer, hepatocellular adenoma, large intestine cancer, endometrial carcinoma or uterine carcinoma, salivary gland tumor, kidney cancer, cervix cancer, prostate cancer, vulvar cancer, thyroid cancer, head and neck cancer, brain cancer, biliary tract cancer, gallbladder cancer, and the like. The cancer may be a primary cancer or a metastatic cancer.

As used herein, the term “prevention and/or treatment of cancer” may refer to cancer cell death, inhibition of cancer cell proliferation, alleviation of symptoms associated with cancer, inhibition of metastasis of cancer, etc.

As used herein, the term “enhancement of immune response” may refer to 4-1BB signal activation, enhancement in any immune response associated with 4-1BB, such as 4-1BB-induced signal activation (e.g., 4-1BB-induced NF-kB signal activation, increase in release of cytokine, target cell killing by immune cells, such as T cells, and the like, but not be limited thereto). In some embodiment, the enhancement of immune response by the bispecific antibody provided by this disclosure may occur be in the presence of HER2.

The pharmaceutical composition may further comprise a pharmaceutically acceptable carrier, diluent, and/or excipient, in addition to the bispecific antibody as an active ingredient. The pharmaceutically acceptable carrier, diluent, and/or excipient may be anyone selected from those commonly used for the formulation of antibodies. For example, the pharmaceutically acceptable carrier may be one or more selected from the group consisting of lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and mineral oil, but are not limited thereto.

The pharmaceutical composition may further comprise one or more selected from the group consisting of a lubricant, a wetting agent, a sweetener, a flavor enhancer, an emulsifying agent, a suspension agent, preservative, and the like.

The bispecific antibody or the pharmaceutical composition may be administered to the subject orally or parenterally. The parenteral administration may be intravenous injection, subcutaneous injection, muscular injection, intraperitoneal injection, endothelial administration, local administration, intranasal administration, intrapulmonary administration, or rectal administration. Since oral administration leads to digestion of proteins or peptides, an active ingredient in the compositions for oral administration must be coated or formulated to prevent digestion in stomach. In addition, the compositions may be administered using an optional device that enables the active ingredient to be delivered to target cells (e.g., cancer cells).

As used herein, the term “the pharmaceutically effective amount” may refer to an amount at which the active ingredient, bispecific antibody, can exert pharmaceutically meaningful effects in preventing or treating cancer. The pharmaceutically effective amount of the bispecific antibody, or a suitable dosage of the pharmaceutical composition indicated by an amount of the bispecific antibody, may be prescribed in a variety of ways, depending on various factors, such as age, body weight, gender, pathologic conditions, diets, excretion speed, and/or reaction sensitivity of a patient, formulation types, administration time, administration route, administration manner, and the like. For example, the pharmaceutically effective amount of the bispecific antibody, or a suitable dosage of the pharmaceutical composition, may be in the range from about 0.001 to about 1000 mg (amount of the bispecific antibody)/kg (body weight), about 0.01 to about 100 mg/kg, or 0.1 to 50 mg/kg per day for an adult.

The subject to which the bispecific antibody or the pharmaceutical composition is administered may be one selected from mammals, for example, humans, monkeys, rats, mice, dogs, cats, guinea pigs, rabbits, rats, mice, horses, cattle, cows, and so on, or a cell or tissue obtained therefrom, but are not limited thereto, and it may be one suffering from cancer.

The pharmaceutical composition formulated with a pharmaceutically acceptable carrier and/or excipient into a unit or a multiple dosage form by a method easily carried out by a skilled person in the pertinent art. The dosage form may be a solution in oil or an aqueous medium, a suspension, syrup, an emulsifying solution, an extract, powder, granules, a tablet, or a capsule, and may further include a dispersing or a stabilizing agent.

### Polynucleotide, Recombinant Vector, and Preparation of Antibody

An embodiment provides a polynucleotide encoding the bispecific antibody. For example, the polypeptide may comprise a first polynucleotide encoding a heavy chain of an anti-Her2 antibody as described herein and a scFv of an anti-4-1BB antibody as described herein, which are linked directly or via a peptide linker; and a second polynucleotide encoding a light chain of the anti-HER2 antibody. Alternatively, the polypeptide may comprise a first polynucleotide encoding a heavy chain of an anti-4-1BB antibody as described herein and a scFv of an anti-HER2 antibody as described herein, which are linked directly or via a peptide linker; and a second polynucleotide encoding a light chain of the anti-4-1BB antibody.

Another embodiment provides a recombinant vector comprising the polynucleotide. For example, the recombinant vector may comprise the first polynucleotide and the second polynucleotide together in one vector or separately in two vectors. Another embodiment provides a recombinant cell comprising the first polynucleotide and the second polynucleotide. For example, the recombinant cell may be a cell transfected with the recombinant vector.

Another embodiment provides a method of preparing the bispecific antibody, comprising expressing the polynucleotide, for example the first polynucleotide and the second polynucleotide, in a cell. The step of expressing the polynucleotide may be conducted by culturing the cell comprising the polynucleotide (for example, in a recombinant vector) under a condition allowing the expression of the polynucleotide. The method may further comprise isolating and/or purifying the anti-4-1BB antibody or an antigen-binding fragment thereof from the cell culture, after the step of expressing or culturing.

The term “vector” refers to a means for expressing a target gene in a host cell, as exemplified by a plasmid vector, a cosmid vector, and a viral vector such as a bacteriophage vector, an adenovirus vector, a retrovirus vector, and an adeno-associated virus vector. The recombinant vector may be constructed from plasmids frequently used in the art (for example, pSC101, pGV1106, pACYC177, ColE1, pKT230, pME290, pBR322, pUC8/9, pUC6, pBD9, pHC79, pIJ61, pLAFR1, pHV14, pGEX series, pET series, and pUC19), phages (for example, λgt4λB, λ-Charon, λΔz1, and M13) or by manipulating viruses (for example, SV40, etc.).

In the recombinant vector, the polynucleotide may be operatively linked to a promoter. The term “operatively linked” is intended to pertain to a functional linkage between a nucleotide sequence of interest and an expression regulatory sequence (for example, a promoter sequence). When being “operatively linked”, the regulatory element can control the transcription and/or translation of the nucleotide of interest.

The recombinant vector may be constructed typically as a cloning vector or an expression vector. For recombinant expression vectors, a vector generally available in the relevant art for expressing a foreign protein in plant, animal, or microbial cells may be employed. Various methods well known in the art may be used for the construction of recombinant vectors.

For use in hosts, such as prokaryotic or eukaryotic cells, the recombinant vector may be constructed accordingly. For example, when a vector is constructed as an expression vector for use in a prokaryotic host, the vector typically includes a strong promoter for transcription (e.g., a pLκλ promoter, a CMV promoter, a trp promoter, a lac promoter, a tac promoter, a T7 promoter, etc.), a ribosomal binding site for initiating translation, and transcriptional/translational termination sequences. On the other hand, an expression vector for use in a eukaryotic host includes an origin of replication operable in a eukaryotic cell, such as an f1 origin of replication, an SV40 origin of replication, a pMB1 origin of replication, an adeno origin of replication, an AAV origin of replication, and a BBV origin of replication, but is not limited thereto. In addition, the expression vector typically includes a promoter derived from genomes of mammalian cells (for example, metallothionein promoter) or from mammalian viruses (for example, adenovirus late promoter, vaccinia virus 7.5K promoter, SV40 promoter, cytomegalovirus promoter, and tk promoter of HSV), and a polyadenylation sequence as a transcription termination sequence.

The recombinant cell may be prepared by introducing the recombinant vector into a suitable host cell. As long as it allows the sequential cloning and expression of the recombinant vector in a stable manner, any host cell known in the art may be employed in the present disclosure. Examples of the prokaryotic host cell available for the present disclosure may be selected from E. coli, Bacillus spp. such as Bacillus subtilis and Bacillus thuringiensis, and enterobacteriaceae strains such as Salmonella typhimurium, Serratia marcescens and various Pseudomonas species. Eukaryotic host cells that may be used for transformation may selected from, but are not limited to, Saccharomyce scerevisiae, insect cells, and animal cells, such as Sp2/0, CHO (Chinese hamster ovary) K1, CHO DG44, PER.C6, W138, BHK, COS-7, 293, HepG2, Huh7, 3T3, RIN, and MDCK.

The polynucleotide or a recombinant vector carrying the same may be introduced (transfected) into a host cell using a method well known in the relevant art. For example, this transfection may be carried out using a CaCl2 or electroporation method when the host cell is prokaryotic. For eukaryotic host cells, the genetic introduction may be achieved using, but not limited to, microinjection, calcium phosphate precipitation, electroporation, liposome-mediated transfection, or particle bombardment.

To select a transformed host cell, advantage may be taken of a phenotype associated with a selection marker according to methods well known in the art. For example, when the selection marker is a gene conferring resistance to a certain antibiotic, the host cells may be grown in the presence of the antibiotic in a medium to select a transformant of interest.

Another embodiment provides a method for production of the bispecific antibody, the method comprising a step of expressing the polynucleotide or the recombinant vector in a host cell. In one embodiment, the production method may comprise culturing a recombinant cell harboring the polynucleotide or the recombinant vector thereat, and optionally isolating and/or purifying the antibody from the culture medium.

### EXAMPLES

Hereafter, the present invention will be described in detail by examples.

The following examples are intended merely to illustrate the invention and are not construed to restrict the invention.

### Example 1. Anti-4-1BB Antibodies

### 1.1 Preparation of Full Human Monoclonal Antibodies Against 4-1BB

Full human monoclonal anti-4-1BB antibodies in a full-length IgG form were screened by phage library immunotube panning against 4-1BB. For panning of the phage library (obtained from KBio Health and CUREBIO) against target molecules, four rounds of panning were carried out in total using 4-1BB (NCBI Accession No. NP_001552.2) coated immunotubes.

Bacterial colonies from the 3 rounds of panning output were grown in SB-Carbenicillin (Biomatik cat#A2311-5g) in 96 deepwell plate until turbid, at which point 10¹¹ pfu of VCSM13 helper phage (K-Bio Health) was added to each well. After 1 h infection at 37° C. with gentle shaking (80 rpm), 70 μg/mL of kanamycin was added and the cells were cultured overnight at 30° C. with shaking at 200 rpm.

Next day, the plates were centrifuged and the supernatants containing the phages were added to 4-1BB antigen-coated ELISA plates blocked with 3% (v/v) BSA (bovine serum albumin) in PBST (Phosphate Buffered Saline with Tween 20). After 1 h incubation at room temperature, the plates were washed three times with PBST and anti M13 antibody (Sino Biological cat#11973-MM05) was added. The plates were incubated for 1 h, washed three times with PBST, and the binding activity was measured using tetramethylbenzidine (TMB).

The 4-1BB specific binders were amplified for plasmid DNA sequencing. The light chain- and heavy chain-variable region (VL and VH) sequences were analyzed to identify unique sequences and determine sequence diversity, as shown in Tables 6 to 13 (Underline: CDR1, CDR2, and CDR3, in order). The anti-4-1BB antibody indicated as BMUR (BMS's Urelumab, U.S. Pat. No. 7,288,638) is used for comparing agonistic activity in following examples.

TABLE 6
1A10

| 1A10 | Amino acid sequence (N′→C′) |
|---|---|
| Heavy Chain | EVQLLESGGGLVQPGGSLRLSCAASGFTFSSYDMSWVRQAP |
| | GKGLEWVSWISYSGGSIYYADSVKGRFTISRDNSKNTLYLQMN |
| | SLRAEDTAVYYCARDGQRNSMREFDYWGQGTLVTVSSASTK |
| | GPSVFPLAPCSRSTSESTAALGCLVKDYFPEPVTVSWNSGALT |
| | SGVHTFPAVLQSSGLYSLSSVVTVPSSSLGTKTYTCNVDHKPS |
| | NTKVDKRVESKYGPPCPPCPAPEFLGGPSVFLFPPKPKDTLMI |
| | SRTPEVTCVVVDVSQEDPEVQFNWYVDGVEVHNAKTKPREE |
| | QFNSTYRVVSVLTVLHQDWLNGKEYKCKVSNKGLPSSIEKTIS |
| | KAKGQPREPQVYTLPPSQEEMTKNQVSLTCLVKGFYPSDIAVE |
| | WESNGQPENNYKTTPPVLDSDGSFFLYSRLTVDKSRWQEGN |
| | VFSCSVMHEALHNHYTQKSLSLSLGK (SEQ ID NO: 56) |
| Heavy Chain | EVQLLESGGGLVQPGGSLRLSCAASGFTFSSYDMSWVRQAP |
| Variable | GKGLEWVSWISYSGGSIYYADSVKGRFTISRDNSKNTLYLQMN |
| Region (VH) | SLRAEDTAVYYCARDGQRNSMREFDYWGQGTLVTVSS(SEQ |
| | ID NO: 18) |
| H-CDR1 | SYDMS (SEQ ID NO: 1) |
| H-CDR2 | WISYSGGSIYYADSVKG (SEQ ID NO: 4) |
| H-CDR3 | DGQRNSMREFDY (SEQ ID NO: 7) |
| Light Chain | QSVLTQPPSASGTPGRRVTISCSGSSSNIGNNYVTWYQQLPG |
| | TAPKLLIYADSHRPSGVPDRFSGSKSGTSASLAISGLRSEDEAD |
| | YYCATWDYSLSGYVFGGGTKLTVLGQPKAAPSVTLFPPSSEEL |
| | QANKATLVCLISDFYPGAVTVAWKADSSPVKAGVETTTPSKQS |
| | NNKYAASSYLSLTPEQWKSHRSYSCQVTHEGSTVEKTVAPAE |
| | CS (SEQ ID NO: 62) |
| Light Chain | QSVLTQPPSASGTPGRRVTISCSGSSSNIGNNYVTWYQQLPGT |
| Variable | APKLLIYADSHRPSGVPDRFSGSKSGTSASLAISGLRSEDEADY |
| Region (VL) | YCATWDYSLSGYVFGGGTKLTVL (SEQ ID NO: 30) |
| L-CDR1 | SGSSSNIGNNYVT (SEQ ID NO: 12) |
| L-CDR2 | ADSHRPS (SEQ ID NO: 14) |
| L-CDR3 | ATWDYSLSGYV (SEQ ID NO: 16) |

TABLE 7
1A10 M4

| 1A10 M4 | Amino acid sequence (N′→C′) |
|---|---|
| Heavy Chain | EVQLLESGGGLVQPGGSLRLSCAASGFTFSSYDMSWVRQAP |
| | GKGLEWVSWISYSGGSIYYADSVKGRFTISRDNSKNTLYLQMN |
| | SLRAEDTAVYYCARDAQRNSMREFDYWGQGTLVTVSSASTK |
| | GPSVFPLAPCSRSTSESTAALGCLVKDYFPEPVTVSWNSGALT |
| | SGVHTFPAVLQSSGLYSLSSVVTVPSSSLGTKTYTCNVDHKPS |
| | NTKVDKRVESKYGPPCPPCPAPEFLGGPSVFLFPPKPKDTLMI |
| | SRTPEVTCVVVDVSQEDPEVQFNWYVDGVEVHNAKTKPREE |
| | QFNSTYRVVSVLTVLHQDWLNGKEYKCKVSNKGLPSSIEKTIS |
| | KAKGQPREPQVYTLPPSQEEMTKNQVSLTCLVKGFYPSDIAVE |
| | WESNGQPENNYKTTPPVLDSDGSFFLYSRLTVDKSRWQEGN |
| | VFSCSVMHEALHNHYTQKSLSLSLGK (SEQ ID NO: 57) |
| Heavy Chain | EVQLLESGGGLVQPGGSLRLSCAASGFTFSSYDMSWVRQAP |
| Variable | GKGLEWVSWISYSGGSIYYADSVKGRFTISRDNSKNTLYLQMN |
| Region (VH) | SLRAEDTAVYYCARDAQRNSMREFDYWGQGTLVTVSS (SEQ |
| | ID NO: 19) |
| H-CDR1 | SYDMS (SEQ ID NO: 1) |
| H-CDR2 | WISYSGGSIYYADSVKG (SEQ ID NO: 4) |
| H-CDR3 | DAQRNSMREFDY (SEQ ID NO: 8) |
| Light Chain | QSVLTQPPSASGTPGRRVTISCSGSSSNIGNNYVTWYQQLPGT |
| | APKLLIYADSHRPSGVPDRFSGSKSGTSASLAISGLRSEDEADY |
| | YCATWDYSLSGYVFGGGTKLTVLGQPKAAPSVTLFPPSSEELQ |
| | ANKATLVCLISDFYPGAVTVAWKADSSPVKAGVETTTPSKQSN |
| | NKYAASSYLSLTPEQWKSHRSYSCQVTHEGSTVEKTVAPAECS |
| | (SEQ ID NO: 62) |
| Light Chain | QSVLTQPPSASGTPGRRVTISCSGSSSNIGNNYVTWYQQLPGT |
| Variable | APKLLIYADSHRPSGVPDRFSGSKSGTSASLAISGLRSEDEADY |
| Region (VL) | YCATWDYSLSGYVFGGGTKLTVL (SEQ ID NO: 30) |
| L-CDR1 | SGSSSNIGNNYVT (SEQ ID NO: 12) |
| L-CDR2 | ADSHRPS (SEQ ID NO: 14) |
| L-CDR3 | ATWDYSLSGYV (SEQ ID NO: 16) |

TABLE 8
1A10 M11

| 1A10 M11 | Amino acid sequence (N′→C′) |
|---|---|
| Heavy Chain | EVQLLESGGGLVQPGGSLRLSCAASGFTFSSYDMSWVRQAP |
| | GKGLEWVSWISYSGGSIYYADSVKGRFTISRDNSKNTLYLQMN |
| | SLRAEDTAVYYCARDAQRQSMREFDYWGQGTLVTVSSASTK |
| | GPSVFPLAPCSRSTSESTAALGCLVKDYFPEPVTVSWNSGALT |
| | SGVHTFPAVLQSSGLYSLSSVVTVPSSSLGTKTYTCNVDHKPS |
| | NTKVDKRVESKYGPPCPPCPAPEFLGGPSVFLFPPKPKDTLMI |
| | SRTPEVTCVVVDVSQEDPEVQFNWYVDGVEVHNAKTKPREE |
| | QFNSTYRVVSVLTVLHQDWLNGKEYKCKVSNKGLPSSIEKTIS |
| | KAKGQPREPQVYTLPPSQEEMTKNQVSLTCLVKGFYPSDIAVE |
| | WESNGQPENNYKTTPPVLDSDGSFFLYSRLTVDKSRWQEGN |
| | VFSCSVMHEALHNHYTQKSLSLSLGK (SEQ ID NO: 58) |
| Heavy Chain | EVQLLESGGGLVQPGGSLRLSCAASGFTFSSYDMSWVRQAP |
| Variable | GKGLEWVSWISYSGGSIYYADSVKGRFTISRDNSKNTLYLQMN |
| Region (VH) | SLRAEDTAVYYCARDAQRQSMREFDYWGQGTLVTVSS (SEQ |
| | ID NO: 20) |
| H-CDR1 | SYDMS (SEQ ID NO: 1) |
| H-CDR2 | WISYSGGSIYYADSVKG (SEQ ID NO: 4) |
| H-CDR3 | DAQRQSMREFDY (SEQ ID NO: 9) |
| Light Chain | QSVLTQPPSASGTPGRRVTISCSGSSSNIGNNYVTWYQQLPGT |
| | APKLLIYADSHRPSGVPDRFSGSKSGTSASLAISGLRSEDEADY |
| | YCATWDYSLSGYVFGGGTKLTVLGQPKAAPSVTLFPPSSEELQ |
| | ANKATLVCLISDFYPGAVTVAWKADSSPVKAGVETTTPSKQSN |
| | NKYAASSYLSLTPEQWKSHRSYSCQVTHEGSTVEKTVAPAECS |
| | (SEQ ID NO: 62) |
| Light Chain | QSVLTQPPSASGTPGRRVTISCSGSSSNIGNNYVTWYQQLPGT |
| Variable | APKLLIYADSHRPSGVPDRFSGSKSGTSASLAISGLRSEDEADY |
| Region (VL) | YCATWDYSLSGYVFGGGTKLTVL (SEQ ID NO: 30) |
| L-CDR1 | SGSSSNIGNNYVT (SEQ ID NO: 12) |
| L-CDR2 | ADSHRPS (SEQ ID NO: 14) |
| L-CDR3 | ATWDYSLSGYV (SEQ ID NO: 16) |

TABLE 9
1A10 M12

| 1A10 M12 | Amino acid sequence (N′→C′) |
|---|---|
| Heavy Chain | EVQLLESGGGLVQPGGSLRLSCAASGFTFSSYDMSWVRQAP |
| | GKGLEWVSWISYSGGSIYYADSVKGRFTISRDNSKNTLYLQMN |
| | SLRAEDTAVYYCARDAQRNSMREFDYWGQGTLVTVSSASTK |
| | GPSVFPLAPCSRSTSESTAALGCLVKDYFPEPVTVSWNSGALT |
| | SGVHTFPAVLQSSGLYSLSSVVTVPSSSLGTKTYTCNVDHKPS |
| | NTKVDKRVESKYGPPCPPCPAPEFLGGPSVFLFPPKPKDTLMI |
| | SRTPEVTCVVVDVSQEDPEVQFNWYVDGVEVHNAKTKPREE |
| | QFNSTYRVVSVLTVLHQDWLNGKEYKCKVSNKGLPSSIEKTIS |
| | KAKGQPREPQVYTLPPSQEEMTKNQVSLTCLVKGFYPSDIAVE |
| | WESNGQPENNYKTTPPVLDSDGSFFLYSRLTVDKSRWQEGN |
| | VFSCSVMHEALHNHYTQKSLSLSLGK (SEQ ID NO: 57) |
| Heavy Chain | EVQLLESGGGLVQPGGSLRLSCAASGFTFSSYDMSWVRQAP |
| Variable | GKGLEWVSWISYSGGSIYYADSVKGRFTISRDNSKNTLYLQMN |
| Region (VH) | SLRAEDTAVYYCARDAQRNSMREFDYWGQGTLVTVSS (SEQ |
| | ID NO: 19) |
| H-CDR1 | SYDMS (SEQ ID NO: 1) |
| H-CDR2 | WISYSGGSIYYADSVKG (SEQ ID NO: 4) |
| H-CDR3 | DAQRNSMREFDY (SEQ ID NO: 8) |
| Light Chain | QSVLTQPPSASGTPGQRVTISCSGSSSNIGNNYVTWYQQLPGT |
| | APKLLIYADSHRPSGVPDRFSGSKSGTSASLAISGLRSEDEADY |
| | YCATWDYSLSGYVFGGGTKLTVLGQPKAAPSVTLFPPSSEELQ |
| | ANKATLVCLISDFYPGAVTVAWKADSSPVKAGVETTTPSKQSN |
| | NKYAASSYLSLTPEQWKSHRSYSCQVTHEGSTVEKTVAPAECS |
| | (SEQ ID NO: 63) |
| Light Chain | QSVLTQPPSASGTPGQRVTISCSGSSSNIGNNYVTWYQQLPG |
| Variable | TAPKLLIYADSHRPSGVPDRFSGSKSGTSASLAISGLRSEDEAD |
| Region (VL) | YYCATWDYSLSGYVFGGGTKLTVL (SEQ ID NO: 31) |
| L-CDR1 | SGSSSNIGNNYVT (SEQ ID NO: 12) |
| L-CDR2 | ADSHRPS (SEQ ID NO: 14) |
| L-CDR3 | ATWDYSLSGYV (SEQ ID NO: 16) |

TABLE 10
1A10 M13

| 1A10 M13 | Amino acid sequence (N′→C′) |
|---|---|
| Heavy Chain | EVQLLESGGGLVQPGGSLRLSCAASGFTFSSYDMSWVRQAP |
| | GKGLEWVSWISYSGGSIYYADSVKGRFTISRDNSKNTLYLQMN |
| | SLRAEDTAVYYCARDAQRQSMREFDYWGQGTLVTVSSASTK |
| | GPSVFPLAPCSRSTSESTAALGCLVKDYFPEPVTVSWNSGALT |
| | SGVHTFPAVLQSSGLYSLSSVVTVPSSSLGTKTYTCNVDHKPS |
| | NTKVDKRVESKYGPPCPPCPAPEFLGGPSVFLFPPKPKDTLMI |
| | SRTPEVTCVVVDVSQEDPEVQFNWYVDGVEVHNAKTKPREE |
| | QFNSTYRVVSVLTVLHQDWLNGKEYKCKVSNKGLPSSIEKTIS |
| | KAKGQPREPQVYTLPPSQEEMTKNQVSLTCLVKGFYPSDIAVE |
| | WESNGQPENNYKTTPPVLDSDGSFFLYSRLTVDKSRWQEGN |
| | VFSCSVMHEALHNHYTQKSLSLSLGK (SEQ ID NO: 58) |
| Heavy Chain | EVQLLESGGGLVQPGGSLRLSCAASGFTFSSYDMSWVRQAP |
| Variable | GKGLEWVSWISYSGGSIYYADSVKGRFTISRDNSKNTLYLQMN |
| Region (VH) | SLRAEDTAVYYCARDAQRQSMREFDYWGQGTLVTVSS (SEQ |
| | ID NO: 20) |
| H-CDR1 | SYDMS (SEQ ID NO: 1) |
| H-CDR2 | WISYSGGSIYYADSVKG (SEQ ID NO: 4) |
| H-CDR3 | DAQRQSMREFDY (SEQ ID NO: 9) |
| Light Chain | QSVLTQPPSASGTPGQRVTISCSGSSSNIGNNYVTWYQQLPGT |
| | APKLLIYADSHRPSGVPDRFSGSKSGTSASLAISGLRSEDEADY |
| | YCATWDYSLSGYVFGGGTKLTVLGQPKAAPSVTLFPPSSEELQ |
| | ANKATLVCLISDFYPGAVTVAWKADSSPVKAGVETTTPSKQSN |
| | NKYAASSYLSLTPEQWKSHRSYSCQVTHEGSTVEKTVAPAECS |
| | (SEQ ID NO: 63) |
| Light Chain | QSVLTQPPSASGTPGQRVTISCSGSSSNIGNNYVTWYQQLPG |
| Variable | TAPKLLIYADSHRPSGVPDRFSGSKSGTSASLAISGLRSEDEAD |
| Region (VL) | YYCATWDYSLSGYVFGGGTKLTVL (SEQ ID NO: 31) |
| L-CDR1 | SGSSSNIGNNYVT (SEQ ID NO: 12) |
| L-CDR2 | ADSHRPS (SEQ ID NO: 14) |
| L-CDR3 | ATWDYSLSGYV (SEQ ID NO: 16) |

TABLE 11
1A12

| 1A12 | Amino acid sequence (N′→C′) |
|---|---|
| Heavy Chain | EVQLLESGGGLVQPGGSLRLSCAASGFTFSGYDMSWVRQAP |
| | GKGLEWVSVIYPDDGNTYYADSVKGRFTISRDNSKNTLYLQMN |
| | SLRAEDAAVYYCAKHGGQKPTTKSSSAYGMDGWGQGTLVTV |
| | SSASTKGPSVFPLAPCSRSTSESTAALGCLVKDYFPEPVTVSW |
| | NSGALTSGVHTFPAVLQSSGLYSLSSVVTVPSSSLGTKTYTCN |
| | VDHKPSNTKVDKRVESKYGPPCPPCPAPEFLGGPSVFLFPPK |
| | PKDTLMISRTPEVTCVVVDVSQEDPEVQFNWYVDGVEVHNAK |
| | TKPREEQFNSTYRVVSVLTVLHQDWLNGKEYKCKVSNKGLPS |
| | SIEKTISKAKGQPREPQVYTLPPSQEEMTKNQVSLTCLVKGFY |
| | PSDIAVEWESNGQPENNYKTTPPVLDSDGSFFLYSRLTVDKSR |
| | WQEGNVFSCSVMHEALHNHYTQKSLSLSLGK (SEQ ID NO: |
| | 59) |
| Heavy Chain | EVQLLESGGGLVQPGGSLRLSCAASGFTFSGYDMSWVRQAP |
| Variable | GKGLEWVSVIYPDDGNTYYADSVKGRFTISRDNSKNTLYLQMN |
| Region (VH) | SLRAEDAAVYYCAKHGGQKPTTKSSSAYGMDGWGQGTLVTV |
| | SS (SEQ ID NO: 21) |
| H-CDR1 | GYDMS (SEQ ID NO: 2) |
| H-CDR2 | VIYPDDGNTYYADSVKG (SEQ ID NO: 5) |
| H-CDR3 | HGGQKPTTKSSSAYGMDG (SEQ ID NO: 10) |
| Light Chain | QSVLTQPPSASGTPGRRVTISCSGSSSNIGNNYVTWYQQLPGT |
| | APKLLIYADSHRPSGVPDRFSGSKSGTSASLAISGLRSEDEADY |
| | YCATWDYSLSGYVFGGGTKLTVLGQPKAAPSVTLFPPSSEELQ |
| | ANKATLVCLISDFYPGAVTVAWKADSSPVKAGVETTTPSKQSN |
| | NKYAASSYLSLTPEQWKSHRSYSCQVTHEGSTVEKTVAPAECS |
| | (SEQ ID NO: 62) |
| Light Chain | QSVLTQPPSASGTPGRRVTISCSGSSSNIGNNYVTWYQQLPGT |
| Variable | APKLLIYADSHRPSGVPDRFSGSKSGTSASLAISGLRSEDEADY |
| Region (VL) | YCATWDYSLSGYVFGGGTKLTVL (SEQ ID NO: 30) |
| L-CDR1 | SGSSSNIGNNYVT (SEQ ID NO: 12) |
| L-CDR2 | ADSHRPS (SEQ ID NO: 14) |
| L-CDR3 | ATWDYSLSGYV (SEQ ID NO: 16) |

TABLE 12
1A12 M1

| 1A12 M1 | Amino acid sequence (N′→C′) |
|---|---|
| Heavy Chain | EVQLLESGGGLVQPGGSLRLSCAASGFTFSGYDMSWVRQAP |
| | GKGLEWVSVIYPDDGNTYYADSVKGRFTISRDNSKNTLYLQMN |
| | SLRAEDTAVYYCAKHGGQKPTTKSSSAYGMDGWGQGTLVTV |
| | SSASTKGPSVFPLAPCSRSTSESTAALGCLVKDYFPEPVTVSW |
| | NSGALTSGVHTFPAVLQSSGLYSLSSVVTVPSSSLGTKTYTCN |
| | VDHKPSNTKVDKRVESKYGPPCPPCPAPEFLGGPSVFLFPPK |
| | PKDTLMISRTPEVTCVVVDVSQEDPEVQFNWYVDGVEVHNAK |
| | TKPREEQFNSTYRVVSVLTVLHQDWLNGKEYKCKVSNKGLPS |
| | SIEKTISKAKGQPREPQVYTLPPSQEEMTKNQVSLTCLVKGFY |
| | PSDIAVEWESNGQPENNYKTTPPVLDSDGSFFLYSRLTVDKSR |
| | WQEGNVFSCSVMHEALHNHYTQKSLSLSLGK (SEQ ID NO: |
| | 60) |
| Heavy Chain | EVQLLESGGGLVQPGGSLRLSCAASGFTFSGYDMSWVRQAP |
| Variable | GKGLEWVSVIYPDDGNTYYADSVKGRFTISRDNSKNTLYLQMN |
| Region (VH) | SLRAEDTAVYYCAKHGGQKPTTKSSSAYGMDGWGQGTLVTV |
| | SS (SEQ ID NO: 22) |
| H-CDR1 | GYDMS (SEQ ID NO: 2) |
| H-CDR2 | VIYPDDGNTYYADSVKG (SEQ ID NO: 5) |
| H-CDR3 | HGGQKPTTKSSSAYGMDG (SEQ ID NO: 10) |
| Light Chain | QSVLTQPPSASGTPGQRVTISCSGSSSNIGNNYVTWYQQLPGT |
| | APKLLIYADSHRPSGVPDRFSGSKSGTSASLAISGLRSEDEADY |
| | YCATWDYSLSGYVFGGGTKLTVLGQPKAAPSVTLFPPSSEELQ |
| | ANKATLVCLISDFYPGAVTVAWKADSSPVKAGVETTTPSKQSN |
| | NKYAASSYLSLTPEQWKSHRSYSCQVTHEGSTVEKTVAPAECS |
| | (SEQ ID NO: 63) |
| Light Chain | QSVLTQPPSASGTPGQRVTISCSGSSSNIGNNYVTWYQQLPG |
| Variable | TAPKLLIYADSHRPSGVPDRFSGSKSGTSASLAISGLRSEDEAD |
| Region (VL) | YYCATWDYSLSGYVFGGGTKLTVL |
| | (SEQ ID NO: 31) |
| L-CDR1 | SGSSSNIGNNYVT (SEQ ID NO: 12) |
| L-CDR2 | ADSHRPS (SEQ ID NO: 14) |
| L-CDR3 | ATWDYSLSGYV (SEQ ID NO: 16) |

TABLE 13
AB41

| AB41 | Amino acid sequence (N′→C′) |
|---|---|
| Heavy Chain | QVQLQQSGAEVIKPGASVKLSCKASGYTFSSYWMHWVRQAP |
| | GQGLEWIGEINPGNGHTNYNEKFKSRATLTGDTSTSTVYMELS |
| | SLRSEDTAVYYCARSFTTARAFAYWGQGTLVTVSSASTKGPS |
| | VFPLAPCSRSTSESTAALGCLVKDYFPEPVTVSWNSGALTSGV |
| | HTFPAVLQSSGLYSLSSVVTVPSSSLGTKTYTCNVDHKPSNTK |
| | VDKRVESKYGPPCPPCPAPEFLGGPSVFLFPPKPKDTLMISRT |
| | PEVTCVVVDVSQEDPEVQFNWYVDGVEVHNAKTKPREEQFN |
| | STYRVVSVLTVLHQDWLNGKEYKCKVSNKGLPSSIEKTISKAK |
| | GQPREPQVYTLPPSQEEMTKNQVSLTCLVKGFYPSDIAVEWE |
| | SNGQPENNYKTTPPVLDSDGSFFLYSRLTVDKSRWQEGNVFS |
| | CSVMHEALHNHYTQKSLSLSLGK (SEQ ID NO: 61) |
| Heavy Chain | QVQLQQSGAEVIKPGASVKLSCKASGYTFSSYWMHWVRQAP |
| Variable | GQGLEWIGEINPGNGHTNYNEKFKSRATLTGDTSTSTVYMELS |
| Region (VH) | SLRSEDTAVYYCARSFTTARAFAYWGQGTLVTVSS (SEQ ID |
| | NO: 23) |
| H-CDR1 | SYWMH (SEQ ID NO: 3) |
| H-CDR2 | EINPGNGHTNYNEKFKS (SEQ ID NO: 6) |
| H-CDR3 | SFTTARAFAY (SEQ ID NO: 11) |
| Light Chain | DIVMTQSPAFLSVTPGEKVTITCRASQTISDYLHWYQQKPDQA |
| | PKLLIKYASQSISGIPSRFSGSGSGTDFTFTISSLEAEDAATYYC |
| | QDGHSFPPTFGQGTKLEIKRTVAAPSVFIFPPSDEQLKSGTASV |
| | VCLLNNFYPREAKVQWKVDNALQSGNSQESVTEQDSKDSTYS |
| | LSSTLTLSKADYEKHKVYACEVTHQGLSSPVTKSFNRGEC |
| | (SEQ ID NO: 64) |
| Light Chain | DIVMTQSPAFLSVTPGEKVTITCRASQTISDYLHWYQQKPDQA |
| Variable | PKLLIKYASQSISGIPSRFSGSGSGTDFTFTISSLEAEDAATYYC |
| Region (VL) | QDGHSFPPTFGQGTKLEIKR (SEQ ID NO: 32) |
| L-CDR1 | RASQTISDYLH (SEQ ID NO: 13) |
| L-CDR2 | YASQSIS (SEQ ID NO: 15) |
| L-CDR3 | QDGHSFPPT (SEQ ID NO: 17) |

1.2. Preparation of scFv Antibodies Against 4-1BB

Anti-4-1BB scFv antibodies with a structure of (N′)-VL-linker-VH-(C′) were prepared using the variable regions of the full human monoclonal antibodies against 4-1BB shown in Tables 6 to 13 of Example 1.1, wherein the amino acid residue “G” at the position 44 of a heavy chain variable region was substituted with “C”, and the amino acid residue “G” at the position 103 of a light chain variable region was substituted with “C”. Such amino acid substitution from “G” to “C” in scFv can contribute to increase in stabilities of bispecific antibodies comprising the scFv as one target-specific moiety. The amino acid sequences of the prepared anti-4-1BB scFvs were illustrated in following Tables 14 to 19, while skilled persons in the art may apply changes or modifications of amino acid sequences in the following embodiments to meet specific purposes, including applying various types of peptide linkers such as (GGGGS)2, (GGGGS)3, (GGGGS)4, or (GS)9.

TABLE 14
1A10 (scFv)

| | Amino acid sequence (N′→C′) |
|---|---|
| Light chain | QSVLTQPPSASGTPGRRVTISCSGSSSNIGNNYVTWYQQLPG |
| variable region | TAPKLLIYADSHRPSGVPDRFSGSKSGTSASLAISGLRSEDEA |
| (VL) | DYYCATWDYSLSGYVFGCGTKLTVL (SEQ ID NO: 33) |
| Linker | GGGGSGGGGSGGGGSGGGGS (SEQ ID NO: 87) |
| Heavy chain | EVQLLESGGGLVQPGGSLRLSCAASGFTFSSYDMSWVRQAP |
| variable region | GKCLEWVSWISYSGGSIYYADSVKGRFTISRDNSKNTLYLQMN |
| (VH) | SLRAEDTAVYYCARDGQRNSMREFDYWGQGTLVTVSS (SEQ |
| | ID NO: 24) |

TABLE 15
1A10 M4 (scFv)

| | Amino acid sequence (N′→C′) |
|---|---|
| Light chain | QSVLTQPPSASGTPGRRVTISCSGSSSNIGNNY |
| variable region | VTWYQQLPGTAPKLLIYADSHRPSGVPDRFSGS |
| (VL) | KSGTSASLAISGLRSEDEADYYCATWDYSLSGY |
| | VFGCGTKLTVL |
| | (SEQ ID NO: 33) |
| Linker | GGGGSGGGGSGGGGSGGGGS |
| | (SEQ ID NO: 87) |
| Heavy chain | EVQLLESGGGLVQPGGSLRLSCAASGFTFSSYD |
| variable region | MSWVRQAPGKCLEWVSWISYSGGSIYYADSVKG |
| (VH) | RFTISRDNSKNTLYLQMNSLRAEDTAVYYCARD |
| | AQRNSMREFDYWGQGTLVTVSS |
| | (SEQ ID NO: 25) |

TABLE 16
1A10 M12 (scFv)

| | Amino acid sequence (N′→C′) |
|---|---|
| Light chain | QSVLTQPPSASGTPGQRVTISCSGSSSNIGNNYV |
| variable region | TWYQQLPGTAPKLLIYADSHRPSGVPDRFSGSKS |
| (VL) | GTSASLAISGLRSEDEADYYCATWDYSLSGYVFG |
| | CGTKLTVL |
| | (SEQ ID NO: 34) |
| Linker | GGGGSGGGGSGGGGSGGGGS |
| | (SEQ ID NO: 87) |
| Heavy chain | EVQLLESGGGLVQPGGSLRLSCAASGFTFSSYDM |
| variable region | SWVRQAPGKCLEWVSWISYSGGSIYYADSVKGRF |
| (VH) | TISRDNSKNTLYLQMNSLRAEDTAVYYCARDAQR |
| | NSMREFDYWGQGTLVTVSS |
| | (SEQ ID NO: 25) |

TABLE 17
1A12 (scFv)

| | Amino acid sequence (N′→C′) |
|---|---|
| Light chain | QSVLTQPPSASGTPGRRVTISCSGSSSNIGNNYV |
| variable region | TWYQQLPGTAPKLLIYADSHRPSGVPDRFSGSKS |
| (VL) | GTSASLAISGLRSEDEADYYCATWDYSLSGYVFG |
| | CGTKLTVL |
| | (SEQ ID NO: 33) |
| Linker | GGGGSGGGGSGGGGSGGGGS |
| | (SEQ ID NO: 87) |
| Heavy chain | EVQLLESGGGLVQPGGSLRLSCAASGFTFSGYDM |
| variable region | SWVRQAPGKCLEWVSVIYPDDGNTYYADSVKGRF |
| (VH) | TISRDNSKNTLYLQMNSLRAEDAAVYYCAKHGGQ |
| | KPTTKSSSAYGMDGWGQGTLVTVSS |
| | (SEQ ID NO: 27) |

TABLE 18
1A12 M1 (scFv)

| | Amino acid sequence (N′→C′) |
|---|---|
| Light chain | QSVLTQPPSASGTPGQRVTISCSGSSSNIGNNYV |
| variable region | TWYQQLPGTAPKLLIYADSHRPSGVPDRFSGSKS |
| (VL) | GTSASLAISGLRSEDEADYYCATWDYSLSGYVFG |
| | CGTKLTVL |
| | (SEQ ID NO: 34) |
| Linker | GGGGSGGGGSGGGGSGGGGS |
| | (SEQ ID NO: 67) |
| Heavy chain | EVQLLESGGGLVQPGGSLRLSCAASGFTFSGYDM |
| variable region | SWVRQAPGKCLEWVSVIYPDDGNTYYADSVKGRF |
| (VH) | TISRDNSKNTLYLQMNSLRAEDTAVYYCAKHGGQ |
| | KPTTKSSSAYGMDGWGQGTLVTVSS |
| | (SEQ ID NO: 28) |

TABLE 19
AB41 (scFv)

| | Amino acid sequence (N′→C′) |
|---|---|
| Light chain | DIVMTQSPAFLSVTPGEKVTITCRASQTISDYLH |
| variable region | WYQQKPDQAPKLLIKYASQSISGIPSRFSGSGSG |
| (VL) | TDFTFTISSLEAEDAATYYCQDGHSFPPTFGCGT |
| | KLEIKR |
| | (SEQ ID NO: 88) |
| Linker | GGGGSGGGGSGGGGSGGGGS |
| | (SEQ ID NO: 67) |
| Heavy chain | QVQLQQSGAEVIKPGASVKLSCKASGYTFSSYWM |
| variable region | HWVRQAPGQCLEWIGEINPGNGHTNYNEKFKSRA |
| (VH) | TLTGDTSTSTVYMELSSLRSEDTAVYYCARSFTT |
| | ARAFAYWGQGTLVTVSS |
| | (SEQ ID NO: 29) |

### 1.3. Antigen Binding Abilities of Anti-4-1BB Antibodies (Full-Length IgG Form) to Human 4-1BB

### (1) Antigen Binding Activity Measured by ELISA

To evaluate the antigen binding activity, the antibody candidates prepared in Example 1.1 were subjected to ELISA test. Briefly, microtiter plates were coated with human 4-1BB-Fc protein (Sino Biological) at 0.1 μg/ml in PBS, 100 μl/well at 4° C. overnight, and then blocked with 100 μl/well of 5% (v/v) BSA. Five-fold dilutions of humanized antibodies (1A10, 1A12, and AB41) starting from 10 μg/ml were added to each well and incubated for 1-2 hours at room temperature (RT). The plates were washed with PBS/Tween and then incubated with goat-anti-human IgG antibody conjugated with Horse Radish Peroxidase (HRP) (Thermo) for 1 hour at RT. After washing, the plates were developed with TMB substrate and analyzed by spectrophotometer at OD 450-630 nm.

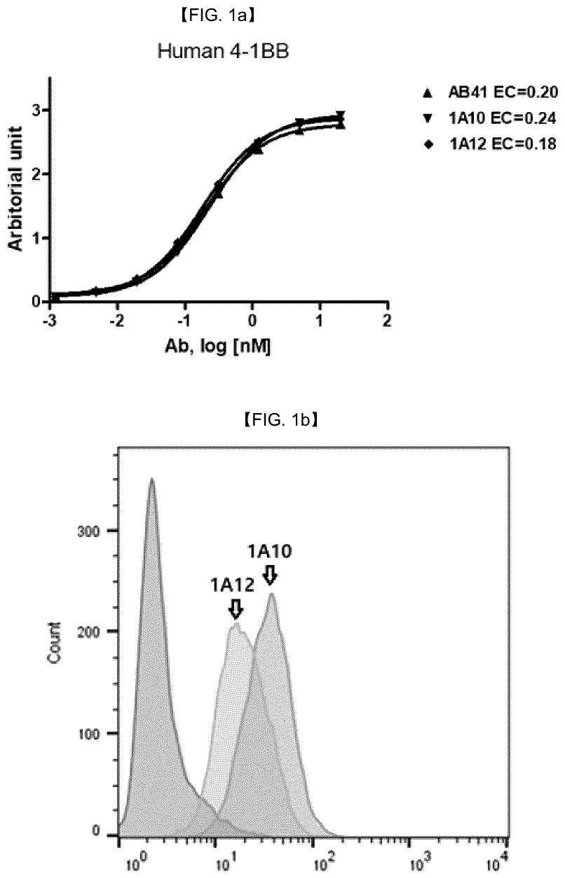

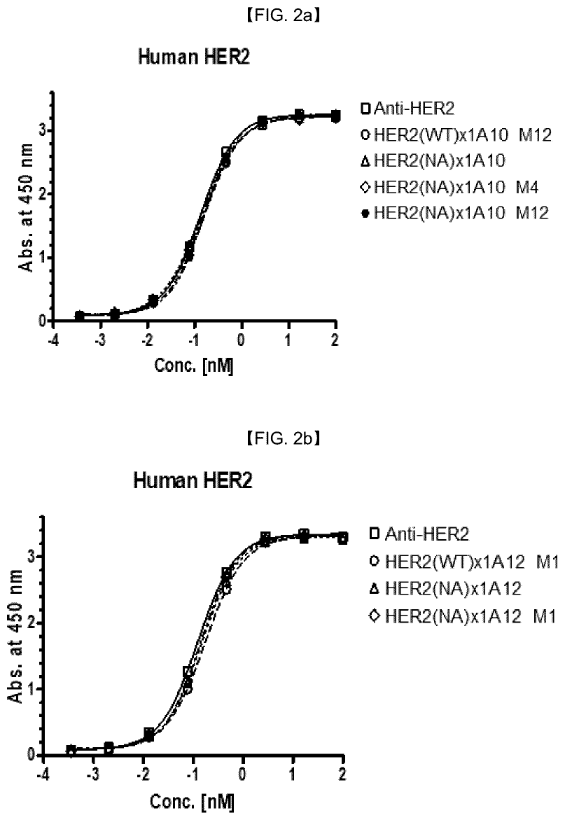

The obtained results are shown in FIG. 1a. As shown in FIG. 1a, all the anti-4-1BB antibodies tested show 4-1BB binding abilities.

### (2) Cell Binding Activity Measured by FACS

To evaluate the cell binding activity, the antibody candidates were analyzed for its binding to mammalian expressed 4-1BB by fluorescence-activated cell sorting (FACS). Briefly, GloResponse™ NFκB-luc2/4-1BB Jurkat cell line (Promega; 3×10⁵ of cells), which are Jurkat cells expressing 4-1BB on their surface, were incubated with antibodies (1A10 and 1A12; each 10 ug/mL). After wash by FACS buffer (1% (v/v) BSA in PBS), the FITC-anti-human IgG antibody (Sigma, F9512, concentration: 2.0 mg/ml) was added to each well and incubated at 4° C. for 1 hour. The mean fluorescence intensity (MFI) of FITC was evaluated by FACSCalibur (BD Biosciences).

The obtained results are shown in FIG. 1b. As shown in FIG. 1b, all the anti-4-1BB antibodies tested show binding abilities to 4-1BB which expressed on cell surface and can efficiently bind to 4-1BB expressed on mammalian cells.

### Example 2. Preparation of Anti-HER2 Antibodies

As a HER2 targeting moiety for anti-HER2/Anti-4-1BB bispecific antibodies, trastuzumab (Genentech; hereinafter indicated as “HER2 (WT)”, DrugBank Accession No. DB00072; human IgG1 Kappa monoclonal antibody), or its antigen-binding fragment, such as scFv, was employed.

The sequences of HER2 (WT) were summarized in following Table 20.

The constant region of the anti-HER2 antibody contained in the bispecific antibody can be modified by introducing more than one mutation or change into human IgG1, one exemplary embodiment, HER2 (NA or N297A) being presented in Table 20 below:

TABLE 20

| HER2(WT) | Sequence |
|---|---|
| Heavy | EVQLVESGGGLVQPGGSLRLSCAASGFNIKDTYIHWVR |
| Chain | QAPGKGLEWVARIYPTNGYTRYADSVKGRFTISADTSK |
| | NTAYLQMNSLRAEDTAVYYCSRWGGDGFYAMDYWGQGT |
| | LVTVSSASTKGPSVFPLAPSSKSTSGGTAALGCLVKDY |
| | FPEPVTVSWNSGALTSGVHTFPAVLQSSGLYSLSSVVT |
| | VPSSSLGTQTYICNVNHKPSNTKVDKKVEPKSCDKTHT |
| | CPPCPAPELLGGPSVFLFPPKPKDTLMISRTPEVTCVV |
| | VDVSHEDPEVKFNWYVDGVEVHNAKTKPREEQYNSTYR |
| | VVSVLTVLHQDWLNGKEYKCKVSNKALPAPIEKTISKA |
| | KGQPREPQVYTLPPSREEMTKNQVSLTCLVKGFYPSDI |
| | AVEWESNGQPENNYKTTPPVLDSDGSFFLYSKLTVDKS |
| | RWQQGNVFSCSVMHEALHNHYTQKSLSLSPGK |
| | (SEQ ID NO: 73) |
| Light Chain | DIQMTQSPSSLSASVGDRVTITCRASQDVNTAVAWYQQ |
| | KPGKAPKLLIYSASFLYSGVPSRFSGSRSGTDFTLTIS |
| | SLQPEDFATYYCQQHYTTPPTFGQGTKVEIKRTVAAPS |
| | VFIFPPSDEQLKSGTASVVCLLNNFYPREAKVQWKVDN |
| | ALQSGNSQESVTEQDSKDSTYSLSSTLTLSKADYEKHK |
| | VYACEVTHQGLSSPVTKSFNRGEC |
| | (SEQ ID NO: 75) |
| HER2(NA) | |
| (N297A) | Sequence |
| Heavy | EVQLVESGGGLVQPGGSLRLSCAASGFNIKDTYIHWVR |
| Chain | QAPGKGLEWVARIYPTNGYTRYADSVKGRFTISADTSK |
| | NTAYLQMNSLRAEDTAVYYCSRWGGDGFYAMDYWGQGT |
| | LVTVSSASTKGPSVFPLAPSSKSTSGGTAALGCLVKDY |
| | FPEPVTVSWNSGALTSGVHTFPAVLQSSGLYSLSSVVT |
| | VPSSSLGTQTYICNVNHKPSNTKVDKKVEPKSCDKTHT |
| | CPPCPAPELLGGPSVFLFPPKPKDTLMISRTPEVTCVV |
| | VDVSHEDPEVKFNWYVDGVEVHNAKTKPREEQYASTYR |
| | VVSVLTVLHQDWLNGKEYKCKVSNKALPAPIEKTISKA |
| | KGQPREPQVYTLPPSREEMTKNQVSLTCLVKGFYPSDI |
| | AVEWESNGQPENNYKTTPPVLDSDGSFFLYSKLTVDKS |
| | RWQQGNVFSCSVMHEALHNHYTQKSLSLSPGK |
| | (SEQ ID NO: 74) |
| Light Chain | DIQMTQSPSSLSASVGDRVTITCRASQDVNTAVAWYQQ |
| | KPGKAPKLLIYSASFLYSGVPSRFSGSRSGTDFTLTIS |
| | SLQPEDFATYYCQQHYTTPPTFGQGTKVEIKRTVAAPS |
| | VFIFPPSDEQLKSGTASVVCLLNNFYPREAKVQWKVDN |
| | ALQSGNSQESVTEQDSKDSTYSLSSTLTLSKADYEKHK |
| | VYACEVTHQGLSSPVTKSFNRGEC |
| | (SEQ ID NO: 75) |

### Example 3. Preparation of Anti-HER2/Anti-4-1BB Bispecific Antibodies

Various anti-HER2/Anti-4-1BB bispecific antibody candidates were prepared in full-length IgG (anti-HER2 antibody)-scFv (anti-4-1BB antibody) format or in full-length IgG (anti-4-1BB antibody)-scFv (anti-HER2 antibody) format: In this example, the anti-HER2 IgG and 4-1BB scFv clones prepared in Example 2 and Example 1.2, respectively, were exemplarily selected, to prepare anti-HER2/anti-4-1BB bispecific antibodies in a IgG-scFv fusion form (an scFv antibody fragment of one antigen is fused to c-terminal of IgG of another antigen). When HER2 is placed in full IgG part, IgG1 with ADCC reduced mutant backbone (N297A mutation; Cancer Cell, vol.19, issue 1, pp.101-113, etc.) was used, and when 4-1BB is placed in full IgG part, IgG4 was used.

A DNA segment 1 having a nucleotide sequence encoding a heavy chain of an IgG antibody of the anti-HER2/anti-4-1BB bispecific antibody was inserted into pcDNA 3.4 (Invitrogen, A14697; plasmid 1), and a DNA segment 2 having a nucleotide sequence encoding a light chain of an IgG antibody of the anti-HER2/anti-4-1BB bispecific antibody was inserted into pcDNA 3.4 (Invitrogen,

A14697; plasmid 2). Thereafter, a DNA segment 3 encoding a scFv was fused at a part of the DNA segment 1 corresponding to the c-terminus of the Fc region of the IgG antibody inserted into the plasmid 1, using a DNA segment 4 encoding a linker peptide having 15 amino acid lengths consisting of (GGGGS)3 or using a DNA segment 5 encoding a linker peptide having 18 amino acid lengths consisting of (GS)9, to construct vectors for the expression of bispecific antibodies. Furthermore, in order to stabilize scFv, as described in Example 1.2, additional modification was applied to generate disulfide bridge fusing VL103-VH44 (VL103: V_{L} having G→C mutation at the position 103; V_{H} 44: V_{H} having G→C mutation at the position 44) to C-terminus of light chain and C-terminus of heavy chain, respectively.

Among the prepared bispecific antibodies, sequences of the heavy chains, light chains, scFvs and DNA segments used in preparing several exemplary bispecific antibodies are illustrated in Tables 21 to 29. One or more than one point mutations in amino acid sequences can be applied in the antibodies presented below, for the purpose of improved stability and potency, decreased immunogenicity, and etc.

TABLE 21
HER2 (NA)x1A10 bispecific antibody-1

| | | | Amino acid sequence (N′→C′) |
|---|---|---|---|
| Heavy | \textcircled{1} Heavy chain | | EVQLVESGGGLVQPGGSLRLSCAASGFNIKDTYIHWVR |
| component | of anti-HER2 | | QAPGKGLEWVARIYPTNGYTRYADSVKGRFTISASTSK |
| | antibody | | NTAYLQMNSLRAEDTAVYYCSRWGGDGFYAMDYWGQGT |
| | | | LVTVSSASTKGPSVFPLAPSSKSTSGGTAALGCLVKDY |
| | | | FPEPVTVSWNSGALTSGVHTFPAVLQSSGLYSLSSVVT |
| | | | VPSSSLGTQTYICNVNHKPSNTKVDKKVEPKSCDKTHT |
| | | | CPPCPAPELLGGPSVFLFPPKPKDTLMISRTPEVTCVV |
| | | | DVSHEDPEVKFNWYVDGVEVHNAKTKPREEQYASTYRV |
| | | | VSVLTVLHQDWLNGKEYKCKVNSKALPAPIEKTISKAK |
| | | | GQPREPQVYTLPPSREEMTKNQVSLTCLVKGFYPSDIA |
| | | | VEWESNGQPENNYKTTPPVLDSDGSFFLYSKLTVDKSR |
| | | | WQQGNVFSCSVMHEALHNHYTQKSLSLSPGK |
| | | | (SEQ ID NO: 74) |
| | \textcircled{2} Linker | | GSGSGSGSGSGSGSGSGS |
| | | | (SEQ ID NO: 85) |
| | \textcircled{3} scFv | VL | QSVLTQPPSASGTPGRVVTISCSGSSSNIGNNYVTWYQ |
| | of anti- | | QLPGTAPKLLIYADSHRPSGVPDRFSGSKSGTSASLAI |
| | 4-1BB | | SGLRSEDEADYYCATWDYSLSGYVFGCGTKLTVL |
| | antibody | | (SEQ ID NO: 33) |
| | | Linker | GGGGSGGGGSGGGGSGGGGS |
| | | | (SEQ ID NO: 87) |
| | | VH | EVQLLESGGGLVQPGGSLRLSCAASGFTFSSYDMSWVR |
| | | | QAPGKCLEWVSWISYSGGSIYYADSVKGRFTISRDNSK |
| | | | NTLYLQMNSLRAEDTAVYYCARDGQRNSMREFDYWGQG |
| | | | TLVTVSS |
| | | | (SEQ ID NO: 24) |
| | Heavy | | EVQLVESGGGLVQPGGSLRLSCAASGFNIKDTYIHWVR |
| | component | | QAPGKGLEWVARIYPTNGYTRYADSVKGRFTISADTSK |
| | (\textcircled{1} + \textcircled{2} + \textcircled{3}) | | NTAYLQMNSLRAEDTAVYYCSRWGGDGFYAMDYWGQGT |
| | | | LVTVSSASTKGPSVFPLAPSSKSTSGGTAALGCLVKDY |
| | | | FPEPVTVSWNSGALTSGVHTFPAVLQSSLGYSLSSVVT |
| | | | VPSSSLGTQTYICNVNHKPSNTKVDKKVEPKSCDKTHT |
| | | | CPPCPAPELLGGPSVFLFPPKPKDTLMISRTPEVTCVV |
| | | | VDVSHEDPEVKDNWYVDGVEVHNAKTKPREEQYASTYR |
| | | | VVSVLTVLHQDWLNGKEYKCKVSNKALPAPIEKTISKA |
| | | | KGQPREPQVYTLPPSREEMTKNQVSLTCLVKGFYPSDI |
| | | | AVEWESNGQPENNYKTTPPVLDSDGSFFLYSKLTVDKS |
| | | | RWQQGNVFSCSVMHEALHNHYTQKSLSLSPGKGSGSGS |
| | | | GSGSGSGSGSGSQSVLTQPPSASGTPGRRVTISCSGSS |
| | | | SNIGNNYVTWYQQLPGTAPKLLIYADSHRPSGVPDRFS |
| | | | GSKSGTSASLAISGLRSEDEADYYCATWDYSLSGYVFG |
| | | | CGTKLTVLGGGGSGGGGSGGGGSGGGGSEVQLLESGGG |
| | | | LVQPGGSLRLSCAASGFTFSSYDMSWVRQAPGKCLEWV |
| | | | SWISYSGGSIYYADSVKGRFTISRDNSKNTLYLQMNSL |
| | | | RAEDTAVYYCARDGQRNSMREFDYWGQGTLVTVSS |
| | | | (SEQ ID NO: 76) |
| Light | Light chain of | | DIQMTQSPSSLSASVGDRVTITCRASQDVNTAVAWYQQ |
| component | anti-HER22 | | KPGKAPKLLIYSASFLYSGVPSRFSGSRSGTDFTLTIS |
| | antibody | | SLQPEDFATYYCQQHYTTPPTFGQGTKVEIKRTVAAPS |
| | | | VFIFPPSDEQLKSGTASVVCLLNNFYPREAKVQWKVDN |
| | | | ALQSGNSQESVTEQDSKDSTYSLSSTLTLSKADYEKHK |
| | | | VYACEVTHQGLSSPVTKSFNRGEC |
| | | | (SEQ ID NO: 75) |

TABLE 22
HER2 (NA)x1A10 bispecific antibody-2

| | | | Amino acid sequence (N′→C′) |
|---|---|---|---|
| Heavy | \textcircled{1} Heavy chain of | | EVQLVESGGGLVQPGGSLRLSCAASGFNIKDTYIHWVR |
| component | anti-HER2 | | QAPGKGLEWVARIYPTNGYTRYADSVKGRFTISADTSK |
| | antibody | | NTAYLQMNSLRAEDTAVYYCSRWGGDGFYAMDYWGQGT |
| | | | LVTVSSASTKGPSVFPLAPSSKSTSGGTAALGCDLVKD |
| | | | YFPEPVTVSWNSGALTSGVHTFPAVLQSSLGYSLSSVV |
| | | | TVPSSSLGTQTYICNVNHKPSNTKVDKKVEPKSCDKTH |
| | | | TCPPCPAPELLGGPSVFLFPPKPKDTLMISRPTEVTCV |
| | | | VVDVSHEDPEVKNFWYVDGVEVHNAKTKPREEQYASTY |
| | | | RVVSVLTVLHQDWLNGKEYKCKVSNKALPAPIEKTISK |
| | | | AKGQPREPQVYTLPPSREEMTKNQVSLTCLVKGFYPSD |
| | | | IAVEWESNGQPENNYKTTPPVLDSDGSFFLYSKLTVDK |
| | | | SRWQQGNVFSCSVMHEALHNHYTQKSLSLSPGK |
| | | | (SEQ ID NO: 74) |
| | \textcircled{2} Linker | | GSGSGSGSGSGSGSGSGS |
| | | | (SEQ ID NO: 86) |
| | \textcircled{3} scFv | VL | QSVLTQPSASGTPGRRVTISCSGSSSNIGNNYVTWYQQ |
| | of anti- | | LPGTAPKLLIYADSHRPSGVPDRFSGSKSGTSASLAIS |
| | 4-1BB | | GLRSEDEADYYCATWDYSLSGYVFGCGTKLTVL |
| | antibody | | (SEQ ID NO: 33) |
| | | Linker | GGGGSGGGGSGGGGSGGGGS |
| | | | (SEQ ID NO: 87) |
| | | VH | EVQLLESGGGLVQPGGSLRLSCAASGFTFSSYDMSWVR |
| | | | QAPGKCLEWVSWISYSGGSIYYADSVKGRFTISRDNSK |
| | | | NTLYLQMNSLRAEDTAVYYCARDGQRNSMREFDYWGQG |
| | | | TLVTVSS |
| | | | (SEQ ID NO: 24) |
| | Heavy component | | EVQLVESGGGLVQPGGSLRLSCAASGFNIKDTYIHWVR |
| | (\textcircled{1} + \textcircled{2} + \textcircled{3}) | | QAPGKGLEWVARIYPTNGYTRYADSVKGRFTISADTSK |
| | | | NTAYLQMNSLRAEDTAVYYCSRWGGDGFYAMDYWGQGT |
| | | | LVTVSSASTKGPSVFPLAPSSKSTSGGTAALGCLVKDY |
| | | | FPEPVTVSWNSGALTSGVHTFPAVLQSSGLYSLSSVVT |
| | | | VPSSSLGTQTYICNVNHKPSNTKVDKKVEPKSCDKTHT |
| | | | CPPCPAPELLGGPSVFLFPPKPKDTLMISRTPEVTCVV |
| | | | VDVSHEDPEVKFNWYVDGVEVHNAKTKPREEQYASTYR |
| | | | VVSVLTVLHQDWLNGKEYKCKVSNKALPAPIEKTISKA |
| | | | KGQPREPQVYTLPPSREEMTKNQVSLTCLVKGFYPSDI |
| | | | AVEWESNGQPENNYKTTPPVLDSDGSFFLYSKLTVDKS |
| | | | RWQQGNVFSCSVMHEALHNHYTQKSLSLSPGKGSGSGS |
| | | | GSGSGSGSGSGQSVLTQPPSASGTPGRRVTISCSGSSS |
| | | | NIGNNYVTWYQQLPGTAPKLLIYADSHRPSGVPDRFSG |
| | | | SKSGTSASLAISGLRSEDEADYYCATWDYSLSGYVFGC |
| | | | GTKLTVLGGGGSGGGGSGGGGSGGGGSEVQLLESGGGL |
| | | | VQPGGSLRLSCAASGFTFSSYDMSWVRQAPGKCLEWVS |
| | | | WISYSGGSIYYADSVKGRFTISRDNSKNTLYLQMNSLR |
| | | | AEDTAVYYCARDGQRNSMREFDYWGQGTLVTVSS |
| | | | (SEQ ID NO: 77) |

| | | |
|---|---|---|
| Light | Light chain of | DIQMTQSPSSLSASVGDRVTITCRASQDVNTAVAWYQQ |
| component | anti-HER2 | KPGKAPKLLIYSASFLYSGVPSRFSGSRSGTDFTLTIS |
| | antibody | SLQPEDFATYYCQQHYTTPPTFGQGTKVEIKRTVAAPS |
| | | VFIFPPSDEQLKSGTASVVCLLNNFYPREAKVQWKVDN |
| | | ALQSGNSQESVTEQDSKDSTYSLSSTLTLSKADYEKHK |
| | | VYACEVTHQGLSSPVTKSFNRGEC |
| | | (SEQ ID NO: 75) |

TABLE 23
HER2 (NA)x1A10 M4 bispecific antibody

| | | | Amino acid sequence (N′→C′) |
|---|---|---|---|
| Heavy | \textcircled{1} Heavy chain | | EVQLVESGGGLVQPGGSLRLSCAASGFNIKDTYIHWVR |
| component | of anti-HER2 | | QAPGKGLEWVARIYPTNGYTRYADSVKGRFTISADTSK |
| | antibody | | NTAYLQMNSLRAEDTAVYYCSRWGGDGFYAMDYWGQGT |
| | | | LVTVSSASTKGPSVFPLAPSSKSTSGGTAALGCLVKDY |
| | | | FPEPVTVSWNSGALTSGVHTFPAVLQSSLGYSLSSVVT |
| | | | VPSSSLGTQTYICNVNHKPSNTKVDKKVEPKSCDKTHT |
| | | | CPPCPAPELLGGPSVFLFPPKPKDTLMISRTPEVTCVV |
| | | | VDVSHEDPEVKFNWYVDGVEVHNAKTKPREEQYASTYR |
| | | | VVSVLTVLHQDWLNGKEYKCKVSNKALPAPIEKTISKA |
| | | | KGQPREPQVYTLPPSREEMTKNQVSLTCLVKGFYPSDI |
| | | | AVEWESNGQPENNKYTTPPVLDSDGSFFLYSKLTVDKS |
| | | | RWQQGNVFSCSVMHEALHNHYTQKSLSLSPGK |
| | | | (SEQ ID NO: 74) |
| | \textcircled{2} Linker | | GSGSGSGSGSGSGSGSGS |
| | | | (SEQ ID NO: 86) |
| | \textcircled{3} scFv | VL | QSVLTQPPSASGTPGRRVTISCSGSSSNIGNNYVTWYQ |
| | of anti- | | QLPGTAPKLLIYADSHRPSGVPDRFSGSKSGTSASLAI |
| | 4-1BB | | SGLRSEDEADYYCATWDYSLSGYVFGCGTKLTVL |
| | antibody | | (SEQ ID NO: 33) |
| | | Linker | GGGGSGGGGSGGGGSGGGGS |
| | | | (SEQ ID NO: 87) |
| | | VH | EVQLLESGGGLVQPGGSLRLSCAASGFTFSSYDMSWVR |
| | | | QAPGKCLEWVSWISYSGGSIYYADSVKGRFTISRDNSK |
| | | | NTLYLQMNSLRAEDTAVYYCARDAQRNSMREFDYWGQG |
| | | | TLVTVSS |
| | | | (SEQ ID NO: 25) |
| | Heavy component | | EVQLVESGGGLVQPGGSLRLSCAASGFNIKDTYIHWVR |
| | (\textcircled{1} + \textcircled{2} + \textcircled{3}) | | QAPGKGLEWVARIYPTNGYTRYADSVKGRFTISASTSK |
| | | | NTAYLQMNSLRAEDTAVYYCSRWGGDGFYAMDYWGQGT |
| | | | LVTVSSASTKGPSVFPLAPSSKSTSGGTAALGCLVKDY |
| | | | FPEPVTVSWNSGALTSGVHTFPAVLQSSLGYSLSSVVT |
| | | | VPSSSLGTQTYICNVNHKPSNTKVDKKVEPKSCDKTHT |
| | | | CPPCPAPELLGGPSVFLFPPKPKDTLMISRTPEVTCVV |
| | | | VDVSHEDPEVKFNWYVDGVEVHNAKTKPREEQYASTYR |
| | | | VVSVLTVLHQDWLNGKEYKCKVSNKALPAPIEKTISKA |
| | | | KGQPREPQVYTLPPSREEMTKNQVSLTLVKGFYPSDIA |
| | | | VEWESNGQPENNYKTTPPVLDSDGSFFLYSKLTVDKSR |
| | | | WQQGNVFSCSVMHEALHNHYTQKSLSLSPGKGSGSGSG |
| | | | SGSGSGSGSGSQSVLTQPPSASGTPGRVVTISCSGSSS |
| | | | NIGNNYVTWYQQLPGTAPKLLIYADSHRPSGVPDWYQQ |
| | | | LPGTAPKLLIYADSHRPSGVPDRFSGSKSGTSASLAIS |
| | | | GLRSEDEADYYCATWDYSLSGYVFGCGTKLTVLGGGGS |
| | | | GGGGSGGGGSGGGGSEVQLLESGGGLVQPGGSLRLSCA |
| | | | ASGFTFSSYDMSWVRQAPGKCLEWVSWISYSGGSIYYA |
| | | | DSVKGRFTISRDNSKNTLYLQMNSLRAEDTAVYYCARD |
| | | | AQRNSMREFDYWGQGTLVTVSS |
| | | | (SEQ ID NO: 78) |
| Light | Light chain of | | DIQMTQSPSSLSASVGDRVTITCRASQDVNTAVAWYQQ |
| component | anti-HER2 | | KPGKAPKLLIYSASFLYSGVPSRFSGSRSGTDFTLTIS |
| | antibody | | SLQPEDFATYYCQQHYTTPPTFGQGTKVEIKRTVAAPS |
| | | | VFIFPPSDEQLKSGTASVVCLLNNFYPREAKVQWKVDN |
| | | | ALQSGNSQESVTEQDSKDSTYSLSSTLTLSKADYEKHK |
| | | | VYACEVTHQGLSSPVTKSFNRGEC |
| | | | (SEQ ID NO: 75) |

TABLE 24
HER2 (NA)x1A10 M12 bispecific antibody

| | | | Amino acid sequence (N′→C′) |
|---|---|---|---|
| Heavy | \textcircled{1} Heavy chain | | EVQLVESGGGLVQPGGSLRLSCAASGFNIKDTYIHWVR |
| component | of anti-HER2 | | QAPGKGLEWVARIYPTNGYTRYADSVKRFTISADTSKN |
| | antibody | | TAYLQMNSLRAEDTAVYYCSRWGGDGFYAMDYWGQGTL |
| | | | VTVSSASTKGPSVFPLAPSSKSTSGGTAALGCLVKDYF |
| | | | PEPVTVSWNSGALTSGVHTFPAVLQSSGLYSLSSVVTV |
| | | | PSSSLGTQTYICNVNHKPSNTKVDKKVEPKSCDKTHTC |
| | | | PPCPAPELLGGPSVFLFPPKPKDTLMISRTPEVTCVVV |
| | | | DVSHEDPEVKFNWYVDGVEVHNAKTKPREEQYASTYRV |
| | | | VSVLTVLHQDWLNGKEYKCKVSNKALPAPIEKTISKAK |
| | | | GQPREPQVYTLPPSREEMTKNQVSLTCLVKGFYPSDIA |
| | | | VEWESNGQPENNYKTTPPVLDSDGSFFLYSKLTVDKSR |
| | | | WQQGNVFSCSVMHEALHNHYTQKSLSLSPGK |
| | | | (SEQ ID NO: 74) |
| | \textcircled{2} Linker | | GSGSGSGSGSGSGSGSGS |
| | | | (SEQ ID NO: 86) |
| | \textcircled{3} scFv | VL | QSVLTQPPSASGTPGQRVTISCSGSSSNIGNNYVTWYQ |
| | of anti- | | QLPGTAPKLLIYADSHRPSGVPDRSGSKSGTSASLAIS |
| | 4-1BB | | GLRSEDEADYYCATWDYSLSGYVFGCGTKLTVL |
| | antibody | | (SEQ ID NO: 34) |
| | | Linker | GGGGSGGGGSGGGGSGGGGS |
| | | | (SEQ ID NO: 87) |
| | | VH | EVQLLESGGGLVQPGGSLRLSCAASGFTFSSYDMSWVR |
| | | | QAPGKCLEWVSWISYSGGSIYYADSVKGRFTISRDNSK |
| | | | NTLYLQMNSLRAEDTAVYYCARDAQRNSMREFDYWGQG |
| | | | TLVTVSS |
| | | | (SEQ ID NO: 25) |
| | Heavy component | | EVQLVESGGGLVQPGGSLRLSCAASGFNIKDTYIHWVR |
| | (\textcircled{1} + \textcircled{2} + \textcircled{3}) | | QAPGKGLEWVARIYPTNGYTRYADSVKGRFTISADTSK |
| | | | NTAYLQMNSLRAEDTAVYYCSRWGGDGFYAMDYWGQGT |
| | | | LVTVSSASTKGPSVFPLAPSSKSTSGGTAALGCLVKDY |
| | | | FPEPVTVSWNSGALTSGVHTFPAVLQSSGLYSLSSVVT |
| | | | VPSSSLGTQTYICNVNHKPSNTKVDKKVEPKSCDKTHT |
| | | | CPPCPAPELLGGPSVFLFPPKPKDTLMISRTPEVTCVV |
| | | | VDVSHEDPEVKFNWYVDGVEVHNAKTKPREEQYASTYR |
| | | | VVSVLTVLHQDWLNGKEYKCKVSNKALPAPIEKTISKA |
| | | | KGQPREPQVYTLPPSREEMTKNQVSLTCLVKGFYPSDI |
| | | | AVEWESNGQPENNYKTTPPVLDSDGSFFLYSKLTVDKS |
| | | | RWQQGNVFSCSVMHEALHNHYTQKSLSLSPGKGSGSGS |
| | | | GSGSGSGSGSGSQSVLTQPPSASGTPGQRVTISCSGSS |
| | | | SNIGNNYVTWYQQLPGTAPKLLIYADSHRPSGVPDRFS |
| | | | GSKSGTSASLAISGLRSEDEADYYCATWDYSLSGYVFG |
| | | | CGTKLTVLGGGGSGGGGSGGGGSGGGGSEVQLLESGGG |
| | | | LVQPGGSLRLSCAASGFTFSSYDMSWVRQAPGKCLEWV |
| | | | SWISYSGGSIYYADSVKGRFTISRDNSKNTLYLQMNSL |
| | | | RAEDTAVYYCARDAQRNSMREFDYWGQGTLVTVSS |
| | | | (SEQ ID NO: 79) |
| Light | Light chain of | | DIQMTQSPSSLSASVGDRVTITCRASQDVNTAVAWYQQ |
| component | anit-HER2 | | KPGKAPKLLIYSASFLYSGVPSRFSGSRSGTDFTLTIS |
| | antibody | | SLPEDFATYYCQQHYTTPPTFGQGTKVEIKRTVAAPSV |
| | | | FIFPPSDEQLKSGTASVVCLLNNFYPREAKVQWKVDNA |
| | | | LQSGNSQESVTEQDSKDSTYSLSSTLTLSKADYEKHKV |
| | | | YACEVTHQGLSSPVTKSFNRGEC |
| | | | (SEQ ID NO: 75) |

TABLE 25
HER2 (NA)x1A12 bispecific antibody-1

| | | | Amino acid sequence (N′→C′) |
|---|---|---|---|
| Heavy | \textcircled{1} Heavy chain | | EVQLVESGGGLVQPGGSLRLSCAASGFNIKDTYIHWVR |
| component | of anti-HER2 | | QAPGKGLEWVARIYPTNGYTRYADSVKGRFTISADTSK |
| | antibody | | NTAYLQMNSLRAEDTAVYYCSRWGGDGFYAMDYWGQGT |
| | | | LVTVSSASTKGPSVFPLAPSSKSTSGGTAALGCLVKDY |
| | | | FPEPVTVSWNSGALTSGVHTFPAVLQSSGLYSLSSVVT |
| | | | VPSSSLGTQTYICNVNHKPSNTKVDKKVEPKSCDKTHT |
| | | | CPPCPAPELLGGPSVFLFPPKPKDTLMISRTPEVTCVV |
| | | | VDVSHEDPEVKFNWYVDGVEVHNAKTKPREEQYASTYR |
| | | | VVSVLTVLHQDWLNGKEYKCKVSNKALPAPIEKTISKA |
| | | | KGQPREPQVYTLPPSREEMTKNQVSLTCLVKGFYPSDI |
| | | | AVEWESNGQPENNYKTTPPVLDSDGSFFLYSKLTVDKS |
| | | | RWQQGNVFSCSVMHEALHNHYTQKSLSLSPGK |
| | | | (SEQ ID NO: 74) |
| | \textcircled{2} Linker | | GGGGSGGGGSGGGGS |
| | | | (SEQ ID NO: 85) |
| | \textcircled{3} scFv | VL | QSVLTQPPSASGTPGRRVTISCSGSSSNIGNNYVTWYQ |
| | of anti- | | QLPGTAPKLLIYADSHRPSGVPDRFSGSKSGTSASLAI |
| | 4-1BB | | SGLRSEDEADYYCATWDYSLSGYVFGCGTKLTVL |
| | antibody | | (SEQ ID NO: 33) |
| | | Linker | GGGGSGGGGSGGGGSGGGGS |
| | | | (SEQ ID NO: 87) |
| | | VH | EVQLLESGGGLVQPGGSLRLSCAASGFTFSGYDMSWVR |
| | | | QAPGKCLEWVSVIYPDDGNTYYADSVKGRFTISRDNSK |
| | | | NTLYLQMNSLRAEDAAVYYCAKHGGQKPTTKSSSAYGM |
| | | | DGWGQGTLVTVSS |
| | | | (SEQ ID NO: 27) |
| | Heavy component | | EVQLVESGGGLVQPGGSLRLSCAASGFNIKDTYIHWVR |
| | (\textcircled{1} + \textcircled{2} + \textcircled{3}) | | QAPGKGLEWVARIYPTNGYTRYADSVKGRFTISADTSK |
| | | | NTAYLQMNSLRAEDTAVYYCSRWGGDGFYAMDYWGQGT |
| | | | LVTVSSASTKGPSVFPLAPSSKSTSGGTAALGCLVKDY |
| | | | FPEPVTVSWNSGALTSGVHTFPAVLQSSGLYSLSSVVT |
| | | | VPSSSLGTQTYICNVNHKPSNTKVDKKVEPKSCDKTHT |
| | | | CPPCPAPELLGGPSVFLFPPKPKDTLMISRTPEVTCVV |
| | | | VDVSHEDPEVKFNWYVDGVEVHNAKTKPREEQYASTYR |
| | | | VVSVLTVLHQDWLNGKEYKCKVSNKALPAPIEKTISKA |
| | | | KGQPREPQVYTLPPSREEMTKNQVSLTCLVKGFYPSDI |
| | | | AVEWESNGQPENNYKTTPPVLDSDGSFFLYSKLTVDKS |
| | | | RWQQGNVFSCSVMHEALHNHYTQKSLSLSPGKGGGGSG |
| | | | GGGSGGGGSQSVLTQPPSASGTPGRRVTISCSGSSSNI |
| | | | GNNYVTWYQQLPGTAPKLLIYADSHRPSGVPDRFSGSK |
| | | | SGTSASLAISGLRSEDEADYYCATWDYSLSGYVFGCGT |
| | | | KLTVLGGGGSGGGGSGGGGSGGGGSEVQLLESGGGLVQ |
| | | | PGGSLRLSCAASGFTFSGYDMSWVRQAPGKCLEWVSVI |
| | | | YPDDGNTYYADSVKGRFTISRDNSKNTLYLMQNSLRAE |
| | | | DAAVYYCAKHGGQKPTTKSSSAYGMDGWGQGTLVTVSS |
| | | | (SEQ ID NO: 80) |
| Light | Light chain of | | DIQMTQSPSSLSASVGDRVTITCRASQDVNTAVAWYQQ |
| component | anti-HER2 | | KPGKAPKLLIYSASFLYSGVPSRFSGSRSGTDFTLTIS |
| | antibody | | SQPEDFATYYCQQHYTTPPTFGQGTKVEIKRTVAAPSV |
| | | | FIFPPSDEQLKSGTASVVCLLNNFYPREAKVQWKVDNA |
| | | | LQSGNSQESVTEQDSKDSTYSLSSTLTLSKADYEKHKV |
| | | | YACEVTHQGLSSPVTKSFNRGEC |
| | | | (SEQ ID NO: 75) |

TABLE 26
HER2 (NA)x1A12 bispecific antibody-2

| | | | Amino acid sequence (N′→C′) |
|---|---|---|---|
| Heavy | \textcircled{1} Heavy chain | | EVQLVESGGGLVQPGGSLRLSCAASGFNIKDTYIHWVR |
| component | of anti-HER2 | | QAPGKGLEWVARIYPTNGYTRYADSVKGRFTISADTSK |
| | antibody | | NTAYLQMNSLRAEDTAVYYCSRWGGDGFYAMDYWGQGT |
| | | | LVTVSSASTKGPSVFPLAPSSKSTSGGTAALGCLVKDY |
| | | | FPEPVTVSWNSGALTSGVHTFPAVLQSSLGYSLSSVVT |
| | | | VPSSSLGTQTYICNVNHKPSNTKVDKKVEPKSCDKTHT |
| | | | CPPCPAPELLGGPSVFLFPPKPKDTLMISRTPECTCVV |
| | | | VDVSHEDPEVKFNWYVDGVEVHNAKTKPREEQYASTYR |
| | | | VVSVLTVLHQDWLNGKEYKCKVSNKALPAPIEKTISKA |
| | | | KGQPREPQVYTLPPSREEMTKNQVSLTCLVKGFYPSDI |
| | | | AVEWESNGQPENNYKTTPPVLDSDGSFFLYSKLTVDKS |
| | | | RWQQGNVFSCSVMHEALHNHYTQKSLSLSPGK |
| | | | (SEQ ID NO: 74) |
| | \textcircled{2} Linker | | GSGSGSGSGSGSGSGSGS |
| | | | (SEQ ID NO: 86) |
| | \textcircled{3} scFv | VL | QSVLTQPPSASGTPGRRVTISCSGSSSNIGNNYVTWYQ |
| | of anti- | | QLPGTAPKLLIYADSHRPSGVPRFSGSKSGTSASLAIS |
| | 4-1BB | | GLRSEDEADYYCATWDYSLSGYVFGCGTKLTVL |
| | antibody | | (SEQ ID NO: 33) |
| | | Linker | GGGGSGGGGSGGGGSGGGGS |
| | | | (SEQ ID NO: 87) |
| | | VH | EVQLLESGGGLVQPGGSLRLSCAASGFTFSGYDMSWVR |
| | | | QAPGKCLEWVSVIYPDDGNTYYADSVKGRFTISRDNSK |
| | | | NTLYLQMNSLRAEDAAVYYCAKHGGQKPTTKSSSAYGM |
| | | | DGWGQGTLVTVSS |
| | | | (SEQ ID NO: 27) |
| | Heavy component | | EVQLVESGGGLVQPGGSLRLSCAASGFNIKDTYIHWVR |
| | (\textcircled{1} + \textcircled{2} + \textcircled{3}) | | QAPGKGLEWVARIYPTNGYTRYADSVKGRFTISADTSK |
| | | | NTAYLQMNSLRAEDTAVYYCSRWGGDGFYAMDYWGQGT |
| | | | LVTVSSASTKGPSVFPLAPSSKSTSGGTAALGCLVKDY |
| | | | FPEPVTVSWNSGALTSGVHTFPAVLQSSGLYSLSSVVT |
| | | | VPSSSLGTQTYICNVNHKPSNTKVDKKVEPKSCDKTHT |
| | | | CPPCPAPELLGGPSVFLFPPKPKDTLMISRTPEVTCVV |
| | | | VDVSHEDPEVKFNWYVDGVEVHNAKTKPREEQYASTYR |
| | | | VVSVLTVLHQDWLNGKEYKCKVSNKALPAPIEKTISKA |
| | | | KGQPREPQVYTLPPSREEMTKNQVSLTCLVKGFYPSDI |
| | | | AVEWESNGQPENNYKTTPPVDLSDGSFFLYSKLTVDKS |
| | | | RWQQGNVFSCSVMHEALHNHYTQKSLSLSPGKGSGSGS |
| | | | GSGSGSGSGSGSQSVLTQPPSASGTPGRRVTISCSGSS |
| | | | SNIGNNYVTWYQQLPGTAPKLLIYADSHRPSGVPDRFS |
| | | | GSKSGTSASLAISGLRSEDEADYYCATWDYSLSGYVFG |
| | | | CGTKLTVLGGGGSGGGGSGGGGSGGGGSEVQLLESGGG |
| | | | LVQPGGSLRLSCAASGFTFSGYDMSWVRQAPGKCLEWV |
| | | | SVIYPDDGNTYYADSVKGRFTISRDNSKNTLYLQMNSL |
| | | | RAEDAAVYYCAKHGGQKPTTKSSSAYGMDGWGQGTLVT |
| | | | VSS |
| | | | (SEQ ID NO: 81) |
| Light | Light chain of | | DIQMTQSPSSLSASVGDRVTITCRASQDVNTAVAWYQQ |
| component | a anti-HER2 | | KPGKAPKLLIYSASFLYSGVPSRFSGSRSGTDFTLTIS |
| | antibody | | SLQPEDFATYYCQQHYTTPPTFGQGTKVEIKRTVAAPS |
| | | | VFIFPPSDEQLKSGTASVVCLLNNFYPREAKVQWKVDN |
| | | | ALQSGNSQESVTEQDSKDSTYSLSSTLTLSKADYEKHK |
| | | | VYACEVTHQGLSSPVTKSFNRGEC |
| | | | (SEQ ID NO: 75) |

TABLE 27
HER2 (NA)x1A12 M1 bispecific antibody

| | | | Amino acid sequence (N′→C′) |
|---|---|---|---|
| Heavy | \textcircled{1} Heavy chain | | EVLQVESGGGLVQPGGSLRLSCAASGFNIKDTYIHWVR |
| component | of anti-HER2 | | QAPGKGLEWVARIYPTNGYTRYADSVKGRFTISADTSK |
| | antibody | | NTAYLQMNSLRAEDTAVYYCSRWGGDGFYAMDYWGQGT |
| | | | LVTVSSASTKGPSVFPLAPSSKSTSGGTAALGCLVKDY |
| | | | FPEPVTVSWNSGALTSGVHTFPAVLQSSGLYSLSSVVT |
| | | | VPSSSLGTQTYICNVNHKPSNTKVDKKVEPKSCDKTHT |
| | | | CPPCPAPELLGGPSVFLFPPKPKDTLMISRTPEVTCVV |
| | | | VDVSHEDPEVKFNWYVDGVEVHNAKTKPREEQYASTYR |
| | | | VVSVLTVLHQDWLNGKEYKCKVSNKALPAPIEKTISKA |
| | | | KGQPREPQVYTLPPSREEMTKNQVSLTCLVKGFYPSDI |
| | | | AVEWESNGQPENNYKTTPPVLDSDGSFFLYSKLTVDKS |
| | | | RWQQGNVFSCSVMHEALHNHYTQKSLSLSPGK |
| | | | (SEQ ID NO: 74) |
| | \textcircled{2} Linker | | GSGSGSGSGSGSGSGSGS |
| | | | (SEQ ID NO: 86) |
| | \textcircled{3} scFv | VL | QSVLTQPPSASGTPGQRVTISCSGSSSNIGNNYVTWYQ |
| | of anti- | | QLPGTAPKLLIYADSHRPSGVPDRFSGSKSGTSASLAI |
| | 4-1BB | | SGLRSEDEADYYCATWDYSLSGYVFGCGTKLTVL |
| | antibody | | (SEQ ID NO: 34) |
| | | Linker | GGGGSGGGGSGGGGSGGGGS |
| | | | (SEQ ID NO: 87) |
| | | VH | EVQLLESGGGLVQPGGSLRLSCAASGFTFSGYDMSWVR |
| | | | QAPGKCLEWVSVIYPDDGNTYYADSVKGRFTISRDNSK |
| | | | NTLYLQMNSLRAEDTAVYYCAKHGGQKPTTKSSSAYGM |
| | | | DGWGQGTLVTVSS |
| | | | (SEQ ID NO: 28) |
| | Heavy component | | EVQLVESGGGLVQPGGSLRLSCAASGFNIKDTYIHWVR |
| | (\textcircled{1} + \textcircled{2} + \textcircled{3}) | | QAPGKGLEWVARIYPTNGYTRYADSVKGRFTISADTSK |
| | | | NTAYLQMNSLRAEDTAVYYCSRWGGDGFYAMDYWGQGT |
| | | | LVTVSSASTKGPSVFPLAPSSKSTSGGTAALGCLVKDY |
| | | | FPEPVTVSWNSGALTSGVHTFPAVLQSSGLYSLSSVVT |
| | | | VPSSSLGTQTYICNVNHKPSNTKVDKKVEPKSCDKTHT |
| | | | CPPCPAPELLGGPSVFLFPPKPKDTLMISRTPEVTCVV |
| | | | VDVSHEDPEVKFNWYVDGVEVHNAKTKPREEQYASTYR |
| | | | VVSVLTVLHQDWLNGKEYKCKVSNKALPAPIEKTISKA |
| | | | KGQPREPQVYTLPPSREEMKTNQVSLTCLVKGFYPSDI |
| | | | AVEWESNGQPENNYKTTPPVLDSDGSFFLYSKLTVDKS |
| | | | RWQQGNVFSCSVMHEALHNHYTQKSLSLSPGKGSGSGS |
| | | | GSGSGSGSGSGSQSVLTQPPSASGTPGQRVTISCSGSS |
| | | | SNIGNNYVTWYQQLPGTAPKLLIYADSHRPSGVPDRFS |
| | | | GSKSGTSASLAISGLRSEDEADYYCATWDYSLSGYVFG |
| | | | CGTKLTVLGGGGSGGGGSGGGGSGGGGSEVQLLESGGG |
| | | | LVQPGGSLRLSCAASGFTFSGYDMSWVRQAPGKCLEWV |
| | | | SVIYPDDGNTYYADSVKGRFTISRDNSKNTLYLQMNSL |
| | | | RAEDTAVYYCAKHGGQKPTTKSSSAYGMDGWGQGTLVT |
| | | | VSS |
| | | | (SEQ ID NO: 82) |
| Light | Light chain of | | DIQMTQSPSSLSASVGDRVTITCRASQDVNTAVAWYQQ |
| component | anti-HER2 | | KPGKAPKLLIYSASFLYSGVPSRFSGSRSGTDFTLTIS |
| | antibody | | SLQPEDFATYYCQQHYTTPPTFGQGTKVEIKRTVAAPS |
| | | | VFIFPPSDEQLKSGTASVVCLLNNFYPREAKVQWKVDN |
| | | | ALQSGNSQESVTEQDSKDSTYSLSSTLTLSKADYEKHK |
| | | | VYACEVTHQGLSSPVTKSFNRGEC |
| | | | (SEQ ID NO: 75) |

TABLE 28
HER2 (WT)x1A10 M12 bispecific antibody

| | | | Amino acid sequence (N′→C′) |
|---|---|---|---|
| Heavy | \textcircled{1} Heavy chain | | EVQLVESGGGLVQPGGSLRLSCAASGFNIKDTYIHWVR |
| component | of anti-HER2 | | QAPGKGLEWVARIYPTNGYTRYADSVKGRFTISADTSK |
| | antibody | | NTAYLQMNSLRAEDTAVYYCSRWGGDGFYAMDYWGQGT |
| | | | LVTVSSASTKGPSVFPLAPSSKSTSGGTAALGCLVKDY |
| | | | FPEPVTVSWNSGALTSGVHTFPAVLQSSGLYSLSSVVT |
| | | | VPSSSLGTQTYICNVNHKPSNTKVDKKVEPKSCDKTHT |
| | | | CPPCPAPELLGGPSVFLFPPKPKDTLMISRTPEVTCVV |
| | | | VDVSHEDPEVKFNWYVDGVEVHNAKTKPREEQYNSTYR |
| | | | VVSVLTVLHQDWLNGKEYKCKVSNKALPAPIEKTISKA |
| | | | KGQPREPQVYTLPPSREEMTKNQVSLTCLVKGFYPSDI |
| | | | AVEWESNGQPENNYKTTPPVDLSDGSFFLYSKLTVDKS |
| | | | RWQQGNVFSCSVMHEALHNHYTQKSLSLSPGK |
| | | | (SEQ ID NO: 73) |
| | \textcircled{2} Linker | | GSGSGSGSGSGSGSGSGS |
| | | | (SEQ ID NO: 86) |
| | \textcircled{3} scFv | VL | QSVLTQPPSASGTPGQRVTISCSGSSSNIGNNYVTWYQ |
| | of anti- | | QLPGTPAKLLIYADSHRPSGVPDRFSGSKSGTSASLAI |
| | 4-1BB | | SGLRSEDEADYYCATWDYSLSGYVFGCGTKLTVL |
| | antibody | | (SEQ ID NO: 34) |
| | | Linker | GGGGSGGGGSGGGGSGGGGS |
| | | | (SEQ ID NO: 87) |
| | | VH | EVQLLESGGGLVQPGGSLRLSCAASGFTFSSYDMSWVR |
| | | | QAPGKCLEWVSWISYSGGSIYYADSVKGRFTISRDNSK |
| | | | NTLYLQMNSLRAEDTAVYYCARDAQRNSMREFDYWGQG |
| | | | TLVTVSS |
| | | | (SEQ ID NO: 25) |
| | Heavy component | | EVQLVESGGGLVQPGGSLRLSCAASGFNIKDTYIHWVR |
| | (\textcircled{1} + \textcircled{2} + \textcircled{3}) | | QAPGKGLEWVARIYPTNGYTRYADSVKGRFTISADTSK |
| | | | NTAYLQMNSLRAEDTAVYYCSRWGGDGFYAMDYWGQGT |
| | | | LVTVSSASTKGPSVFPLAPSSKSTSGGTAALGCLVKDY |
| | | | FPEPVTVSWNSGALTSGVHTFPAVLQSSGLYSLSSVVT |
| | | | VPSSSLGTQTYICNVNHKPSNTKVDKKVEPKSCDKTHT |
| | | | CPPCPAPELLGGPSVFLFPPKPKDTLMISRTPEVTCVV |
| | | | VDVSHEDPEVKFNWYVDGVEVHNAKTKPREEQYNSTYR |
| | | | VVSVLTVLHQDWLNGKEYKCKVSNKALPAPIEKTISKA |
| | | | KGQPREPQVYTLPPSREEMTKNQVSLTCLVKGFYPSDI |
| | | | AVEWESNGQPENNYKTTPPVLDSDGSFFLYSKLTVDKS |
| | | | RWQQGNVFSCSVMHEALHNHYTQKSLSLSPGKGSGSGS |
| | | | GSGSGSGSGSGSQSVLTQPPSASGTPGQRVTISCSGSS |
| | | | SNIGNNYVTWYQQLPGTAPKLLIYADHSRPSGVPDRFS |
| | | | GSKSGTSASLAISGLRSEDEADYYCATWDYSLSGYVFG |
| | | | CGTKLTVLGGGGSGGGGSGGGGSGGGGSEVQLLESGGG |
| | | | LVQPGGSLRLSCAASGFTFSSYDMSWVRQAPGKCLEWV |
| | | | SWISYSGGSIYYADSVKGRFTISRDNSKNTLYLQMNSL |
| | | | RAEDTAVYYCARDAQRNSMREFDYWGQGLTVTVSS |
| | | | (SEQ ID NO: 83) |
| Light | Light chain of | | DIQMTQSPSSLSASVGDRVTITCRASQDVNTAVAWYQQ |
| component | anti-HER2 | | KPGKAPKLLIYSASFLYSGVPSRFSGSRSGTDFTLTIS |
| | antibody | | SLQPEDFATYYCQQHYTTPPTFGQGTKVEIKRTVAAPS |
| | | | VFIFPPSDEQLKSGTASVVCLLNNFYPREAKVQWKVDN |
| | | | ALQSGNSQESVTEQDSKDSTYSLSSTLTLSKADYEKHK |
| | | | VYACEVTHQGLSSPVTKSFNRGEC |
| | | | (SEQ ID NO: 75) |

TABLE 29
HER2 (WT)x1A12 M1 bispecific antibody

| | | | Amino acid sequence (N′→C′) |
|---|---|---|---|
| Heavy | \textcircled{1} Heavy chain | | EVQLVESGGGLVQPGGSLRLSCAASGFNIKDTYIHWVR |
| component | of anti-HER2 | | QAPGKGLEWVARIYPTNGYTRYADSVKGRFTISADTSK |
| | antibody | | NTAYLQMNSLRAEDTAVYYCSRWGGDGFYAMDYWGQGT |
| | | | LVTVSSASTKGPSVFPLAPSSKSTSGGTAALGCLVKDY |
| | | | FPEPVTVSWNSGALTSGVHTFPAVLQSSGLYSLSSVVT |
| | | | VPSSSLGTQTYICNVNHKPSNTKVDKKVEPKSCDKTHT |
| | | | CPPCPAPELLGGPSVFLFPPKPKDTLMISRTPEVTCVV |
| | | | VDVSHEDPEVKFNWYVDGVEVHNAKTKPREEQYNSTYR |
| | | | VVSVLTVLHQDWLNGKEYKCKVSNKALPAPIEKTISKA |
| | | | KGQPREPQVYTLPPSREEMTKNQVSLTCLVKGFYPSDI |
| | | | AVEWESNGQPENNYKTTPPVLDSDGSFFLYSKLTVDKS |
| | | | RWQQGNVFSCSVMHEALHNHYTQKSLSLPGK |
| | | | (SEQ ID NO: 73) |
| | \textcircled{2} Linker | | GSGSGSGSGSGSGSGSGS |
| | | | (SEQ ID NO: 86) |
| | \textcircled{3} scFv | VL | QSVLTQPPSASGTPGQRVTISCSGSSSNIGNNYVTWYQ |
| | of anti- | | QLPGTAPKLLIYADSHRPSGVPDRFSGSKSGTSASLAI |
| | 4-1BB | | SGLRSEDEADYYCATWDYSLSGYVFGCGTKLTVL |
| | antibody | | (SEQ ID NO: 34) |
| | | Linker | GGGGSGGGGSGGGGSGGGGS |
| | | | (SEQ ID NO: 87) |
| | | VH | EVQLLESGGGLVQPGGSLRLSCAASGFTFSGYDMSWVR |
| | | | QAPGKCLEWVSVIYPDDGNTYYADSVKGRFTISRDNSK |
| | | | NTLYLQMNSLRAEDTAVYYCAKHGGQKPTTKSSSAYGM |
| | | | DGWGQGTLVTVSS |
| | | | (SEQ ID NO: 28) |
| | Heavy component | | EVQLVESGGGLVQPGGSLRLSCAASGFNIKDTYIHWVR |
| | (\textcircled{1} + \textcircled{2} + \textcircled{3}) | | QAPGKGLEWVARIYPTNGYTRYADSVKGRFTISADTSK |
| | | | NTAYLQMNSLRAEDTAVYYCSRWGGDGFYAMDYWGQGT |
| | | | LVTVSSASTKGPSVFPLAPSSKSTSGGTAALGCLVKDY |
| | | | FPEPVTVSWNSGALTSGVHTFPAVLQSSGLYSLSSVVT |
| | | | VPSSSLGTQTYICNVNHKPSNTKVDKKVEPKSCDKTHT |
| | | | CPPCPAPELLGGPSVFLFPPKPKDTLMISRTPECTCVV |
| | | | VDVSHEDPEVKFNWYVDGVEVHNAKTKPREEQYNSTYR |
| | | | VVSVLTVLHQDWLNGKEYKCKVSNKALPAPIEKTISKA |
| | | | KGQPREPQVYTLPPSREEMTKNQVSLTCLVKGFYPSDI |
| | | | AVEWESNGQPENNKYTTPPVLDSDGSFFLYSKLTVDKS |
| | | | RWQQGNVFSCSVMHEALHNHYTQKSLSLSPGKGSGSGS |
| | | | GSGSGSGSGSGSQSVLTQPPSASGTPGQRVTISCSGSS |
| | | | SNIGNNYVTWYQQLPGTAPKLLIYADSHRPSGVPDRFS |
| | | | GSKSGTSASLAISGLRSEDEADYYCATWDYSLSGYVFG |
| | | | CGTKLTVLGGGGSGGGGSGGGGSGGGGSEVQLLESGGG |
| | | | LVQPGGSLRLSCAASGFTFSGYDMSWVRQAPGKCLEWV |
| | | | SVIYPDDGNTYYADSVKGRFTISRDNSKNTLYLQMNSL |
| | | | RAEDTAVYYCAKHGGQKPTTKSSSAYGMDGWGQGTLVT |
| | | | VSS |
| | | | (SEQ ID NO: 84) |
| Light | Light chain of | | DIQMTQSPSSLSASVGDRVTITCRASQDVNTAVAWYQQ |
| component | anti-HER2 | | KPGKAPKLLIYSASFLYSGVPSRFSGSRSGTDFTLTIS |
| | antibody | | SLQPEDFATYYCQQHYTTPPTFGQGTKVEIKRTVAAPS |
| | | | VFIFPPSDEQLKSGTASVVCLLNNFYPREAKVQWKVDN |
| | | | ALQSGNSQESVTEQDSKDSTYSLSSTLTLSKADYEKHK |
| | | | VYACEVTHQGLSSPVTKSFNRGEC |
| | | | (SEQ ID NO: 75) |

### Example 4. Test of Binding Affinity of Bispecific Antibodies (BsAbs)

### 4.1. Binding to Human HER2

The HER2 binding affinity of the bispecific antibodies were conducted by ELISA referring to Example 1.3(1). In brief, 96-well microtiter plates (Nunc-Immuno Plates, NUNC) were coated with human HER2-His protein (Sino Biological, 10004-H08H) at 1 μg/ml in PBS, 100 μl/well at 4° C. overnight, and then blocked with blocking buffer (200 μl/well of 1% (v/v) BSA (bovine serum albumin (Gibco, 30063572)) in PBS) for 2 hours at 37° C. Serial dilutions (starting from 0.1 μM) of anti-HER2/anti-4-1BB bispecific antibodies prepared in Example 3 and anti-HER2 antibody (HER2(NA)) as a control were added to each well and incubated for 1 hour at 37° C. The plates were washed with PBS/0.05% Tween20 and incubated with HRP-conjugated Fab antibody (Pierce, 31414) for 1 hour at 37° C. After washing, the plates were developed with TMB (Tetramethylbenzidine, Sigma, T0440) substrate and analyzed by spectrophotometer at OD 450-650 nm.

The obtained results are shown in FIGS. 2a and 2b. As shown in FIGS. 2a and 2b, all the anti-HER2/anti-4-1BB bispecific antibodies tested can bind to human HER2 proteins with high affinity, which is similar to that of the control anti-HER2 antibody (NA).

### 4.2. Binding to Human 4-1BB

The 4-1BB binding affinity of the bispecific antibodies were conducted by ELISA referring to Example 1.3(1). Briefly, 96-well microtiter plates (Nunc-Immuno Plates, NUNC) were coated with human 4-1BB-His protein (Sino Biological, 10041-H08H) at 1 μg/ml in PBS, 100 uμl/well at 4° C. overnight, and then blocked with blocking buffer with blocking buffer (200 μl/well of 1% (v/v) BSA (bovine serum albumin (Gibco, 30063572)) in PBS) for 2 hours at 37° C. Serial dilutions (starting from 0.1 μM) of anti-HER2/anti-4-1BB bispecific antibodies prepared in Example 3 and anti-HER2 antibody (HER2(NA)) as a control were added to each well and incubated for 1 hour at 37° C. The plates were washed with PBS/0.05% Tween20 and incubated with HRP-conjugated Fab antibody (Pierce, 31414) for 1 hour at 37° C. After washing, the plates were developed with TMB substrate and analyzed by spectrophotometer at OD 450-650 nm.

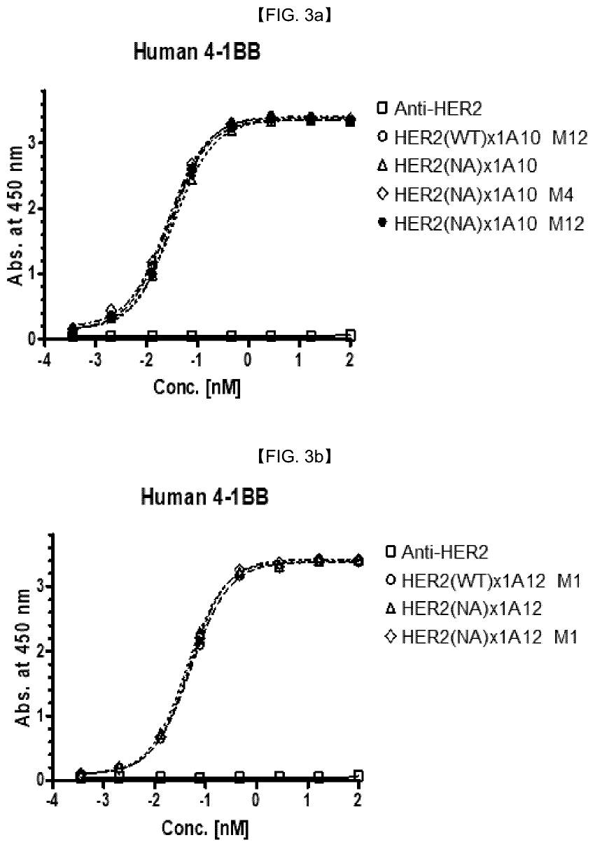

The obtained results are shown in FIGS. 3a and 3b. As shown in FIGS. 3a and 3b, all the anti-HER2/anti-4-1BB bispecific antibodies tested can bind to human 4-1BB protein with high affinity, whereas the anti-HER2 antibody does not bind to human 4-1BB protein.

The results of FIGS. 2a, 2b, 3a, and 3b are quantified and summarized in following Table 30:

TABLE 30

| EC50 (nM) | HER2 | 4-1BB |
|---|---|---|
| HER2(NA) | 0.134 | N/A |
| HER2(WT)x1A10 M12 | 0.159 | 0.027 |
| HER2(NA)x1A10 | 0.135 | 0.033 |
| HER2(NA)x1A10 M4 | 0.148 | 0.025 |
| HER2(NA)x1A10 M12 | 0.161 | 0.031 |
| HER2(WT)x1A12 M1 | 0.174 | 0.051 |
| HER2(NA)x1A12 | 0.148 | 0.043 |
| HER2(NA)x1A12 M1 | 0.143 | 0.048 |

As shown in Table 30, all the anti-HER2/anti-4-1BB bispecific antibodies tested can bind to both human HER2 and human 4-1BB proteins with high affinities.

### 4.3. Binding to Various Cell Surface Expressed Human HER2

The binding affinities of the bispecific antibodies to various cells expressing HER2 on their surface were conducted by FACS analysis referring to Example 1.3(2).

Various tumor cell lines as listed in Table 31 were used. After disassociating each cell lines and washing in PBS, the number of cells was counted and set as 2×10⁵ cells/100 μl FACS buffer, and then anti-HER2 antibody or anti-HER2/anti-4-1BB bispecific antibodies were treated in 10 μg/mL, and they were reacted at 4° C. for 1 hour. After reaction, cells were washed in FACS buffer, and then the FITC labeled constant region (Fc)-specific antibody (Goat anti-human IgG FITC conjugate, Fc specific, Sigma, F9512, concentration: 2.0 mg/ml) was suspended in 2 μl/2×10⁵ cells/100 μl FACS buffer, and it was reacted at 4° C. for 1 hour. After reaction, cells were washed in FACS buffer, it was analyzed using a FACSCalibur device. The negative control group was treated only with the FITC-labeled constant region (Fc)-specific antibody. To compare the expression degrees of HER2 among the cancer cell lines, the value of the result for the peak shift in the experimental group was divided by the result for the peak shift in the negative control group (Mean Fluorescence intensity Ratio=MFI Ratio: MFI of test antibody/MFI of 2nd Ab).

The obtained results are shown in following Table 31:

TABLE 31
MFI ratio

| | | | anti- | | |
|---|---|---|---|---|---|
| Cell line | | | HER2 | HER2x1A10 | HER2x1A12 |
| NCI-N87 | Gastric | ATCC, | 104 | 139 | 145 |
| | | CRL-5822 | | | |
| BT-474 | Breast | ATCC, HTB-20 | 81 | 102 | 90 |
| Calu-3 | Lung | ATCC, HTB-55 | 74 | 82 | 82 |
| HCC1954 | Breast | ATCC, | 32 | 40 | 42 |
| | | CRL-2338 | | | |
| JIMT1 | Breast | DSMZ, | 26 | 27 | 25 |
| | | ACC 589 | | | |
| HT29 | Colon | ATCC, HTB-38 | 6.1 | 6.0 | 7.4 |
| MCF-7 | Breast | ATCC, HTB-22 | 5.2 | 4.9 | 5.8 |
| MDA- | Breast | ATCC, HTB-26 | 1.2 | 1.4 | 1.6 |
| MB231 | | | | | |
| H929 | MM | ATCC, | 0.9 | 1.4 | 1.4 |
| | | CRL-9068 | | | |
| Jurkat | ALL | ATCC, TIB-152 | 1.0 | 1.3 | 1.6 |

(MFI Ratio: MFI of 1^{st} Ab/MFI of 2^{nd} Ab)

As shown in Table 31, all the anti-HER2/anti-4-1BB bispecific antibodies tested can bind to cell surface expressed human HER2 proteins.

### Example 5. Binding Affinity of BsAbs to 4-1BB (SPR)

In the SPR experiment, the anti-HER2/anti-4-1BB bispecific antibodies were individually captured on flow-cells 2, 3 and 4, keeping the flow-cell 1 as reference, on a Biocore® Series S Sensor Chip CM5 (GE Healthcare, BR100530) on which an anti-human Fab antibody (GE Healthcare, 28958325) had been immobilized by amine coupling. Recombinant Human 4-1BB protein (ACROBiosystems, 41B-H5227) was flowed across the chip at concentration of 400, 200, 100, 50, 25, 12.5, 6.25, 3.13, 1.56 and 0.78 nM at 30 μl/min for 300 seconds, followed by a dissociation phase of 400 seconds. Regeneration was performed with 10 mM Glycine-HCl (pH 2.0) (GE Healthcare, BR100355).

The obtained results are shown in following Table 32:

TABLE 32

| Antibody | KD (M) | kon(1/Ms) | kdis(1/s) |
|---|---|---|---|
| HER2(WT)x1A10 M12 | 2.11E−09 | 2.55E+05 | 5.38E−04 |
| HER2(NA)x1A10 M12 | 2.35E−09 | 2.69E+05 | 6.33E−04 |
| HER2(WT)x1A12 M1 | 1.24E−08 | 6.57E+04 | 8.11E−04 |
| HER2(NA)x1A12 M1 | 1.38E−08 | 6.74E+04 | 9.27E−04 |

As shown in Table 32, the anti-HER2/anti-4-1BB bispecific antibodies tested show high 4-1BB binding affinities.

### Example 6. 4-1BB Signal Activation

6.1. BsAbs vs. Monospecific Antibodies

In this example, for measuring 4-1BB signal activation, GloResponse™ NFκB-luc2/4-1BB Jurkat cell line (Promega), genetically modified to stably express human 4-1BB and luciferase downstream of a response element, was used as effector cell and cancer cells expressing or not expressing HER2 were used as target cells. In brief, as cancer cells, NCI-N87 (expressing HER2; 2.5×10⁴ cells) or MDA-MB-231 (not expressing HER2; 2.5×10⁴ cells) were plated in a 96-well assay plate and cultured overnight. On the day of assay, the anti-HER2/anti-4-1BB bispecific antibodies (Example 3) to be tested and effector Jurkat cells (2.5×10⁴ cells) were added to the plate. After 6 hrs incubation, Bio-Glo™ Reagent (Promega) was added and luminescence was measured using a microplate reader.

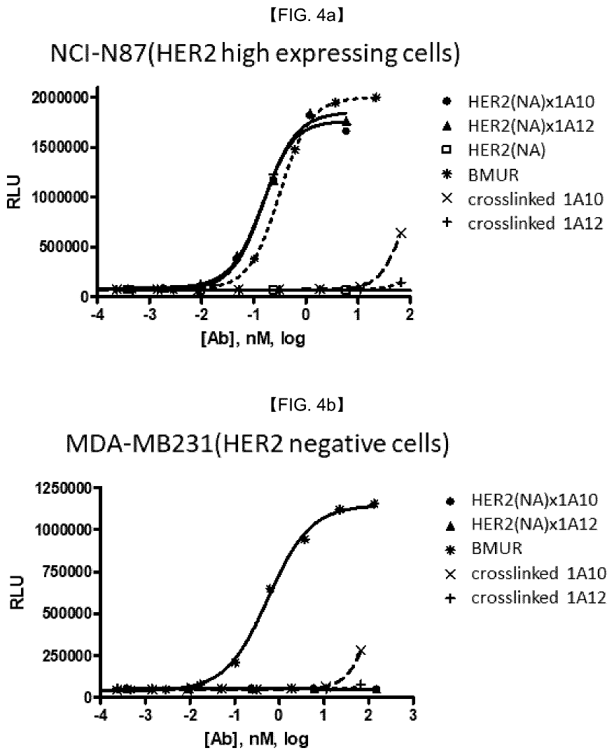

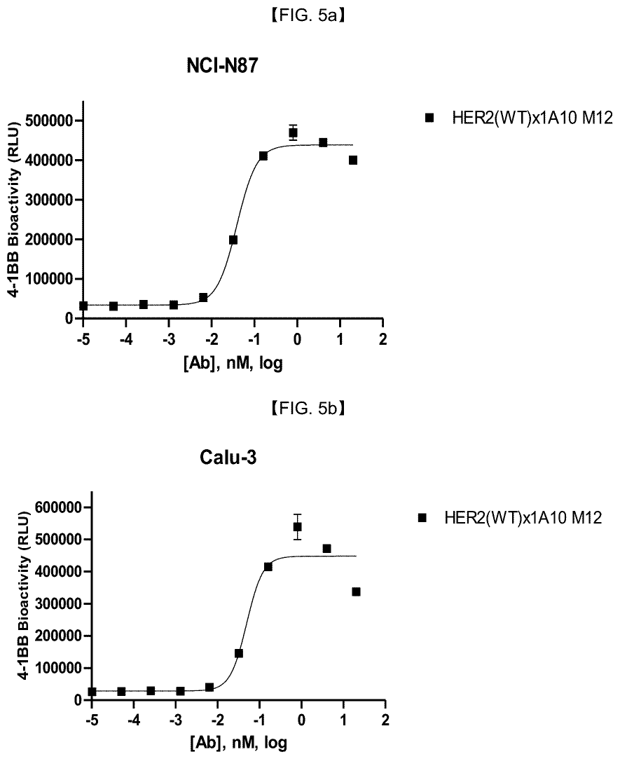

The obtained results are shown in following FIGS. 4a (NCI-N87 cell line) and 4b (MDA-MB-231 cell line). In FIGS. 4a and 4b, BMUR (BMS's Urelumab, U.S. Pat. No. 7,288,638) indicates an anti-4-1BB antibody used for comparing agonistic activity. As shown in FIGS. 4a and 4b, anti-HER2/anti-4-1BB bispecific antibodies lead to strong activation of 4-1BB signal only when co-cultured with HER2 high expressing cell. Fc crosslinked anti-4-1BB monoclonal antibodies showed minimal activity.

### 6.2. 4-1BB Activation in Various HER2-Expressing Cells (I)

In this example, for measuring 4-1BB signal activation, GloResponse™ NFκB-luc2/4-1BB Jurkat cell line (Promega), genetically modified to stably express human 4-1BB and luciferase downstream of a response element, was used as effector cell, and cancer cells expressing or not expressing HER2 were used as target cells. In brief, HER2-expressing (NCI-N87, Calu-3, HCC1954, JIMT1, ZR-75-1) or HER2-non-expressing (MDA-MB231, MCF-7, A431, BxPC-3) cancer cells (each 2.5×10⁴ cells/well) were plated in a 96-well assay plate and cultured overnight. On the day of assay, the anti-HER2/anti-4-1BB bispecific antibodies (Example 3; 15 nM. 4-fold or 20 nM, 5-fold or 100 nM, 5-fold dilution) to be tested and effector Jurkat cells (each 2.5×10⁴ cells/well) were added to the plate. After 6 hours of incubation, Bio-Glo™ Reagent was added and luminescence was measured using a microplate reader.

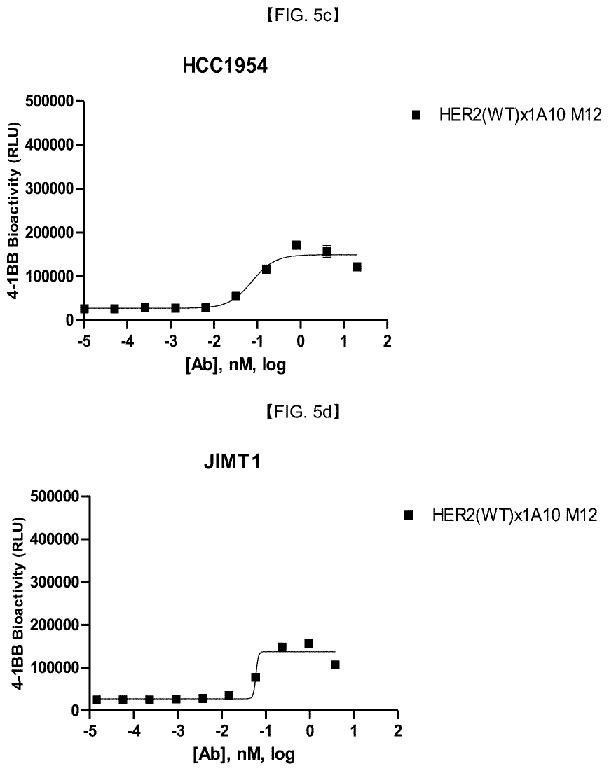

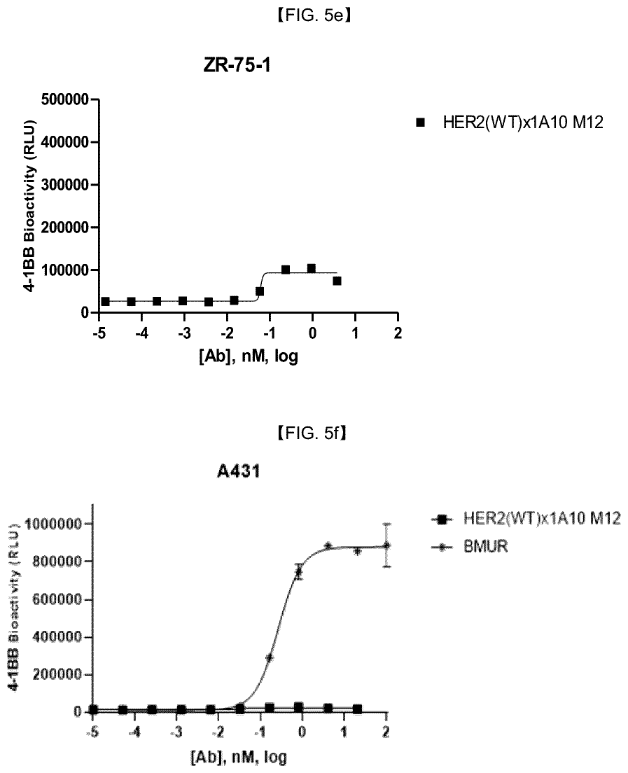

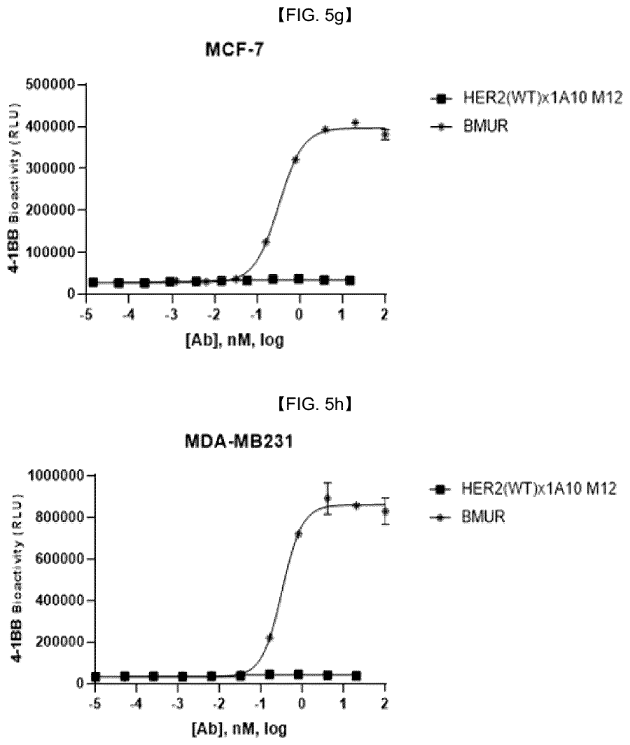

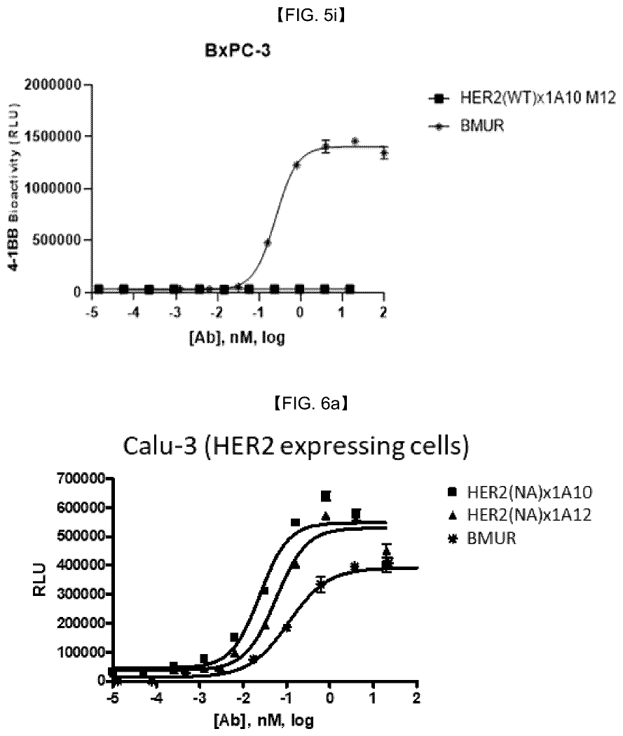

The obtained results are shown in FIGS. 5a-5i. As shown in FIGS. 5a-5i, anti-HER2/anti-4-1BB bispecific antibodies lead to strong activation of 4-1BB signal only when co-cultured with HER2-expressing cells.

### 6.3. 4-1BB Activation in Various HER2-Expressing Cells (II)

In this example, for measuring 4-1BB signal activation, GloResponse™ NFκB-luc2/4-1BB Jurkat cell line (Promega), genetically modified to stably express human 4-1BB and luciferase downstream of a response element, was used as effector cell. In brief, HER2-expressing target cells (Calu-3 or HCC1954; 2.5×10⁴ cells/well) were plated in a 96-well assay plate and cultured overnight. On the day of assay, the anti-HER2/anti-4-1BB bispecific antibodies (Example 3; 20 nM, 5-fold or 133 nM, 6-fold dilution) to be tested and effector Jurkat cells (2.5×10⁴ cells/well) were added to the plate. After 6 hours incubation, Bio-Glo™ Reagent was added and luminescence was measured using a microplate reader.

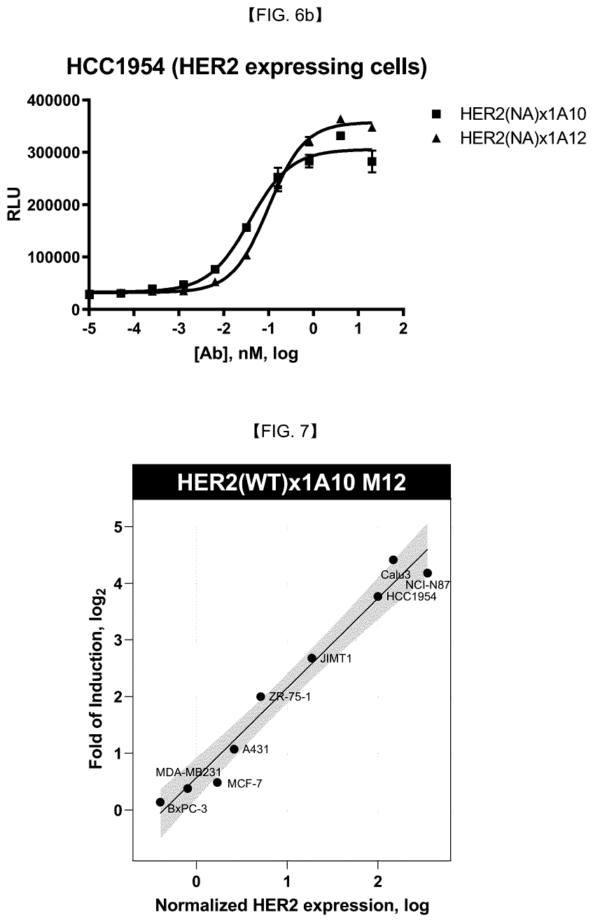

The obtained results are shown in FIGS. 6a and 6b. As shown in FIGS. 6a and 6b, all the anti-HER2/anti-4-1BB bispecific antibodies tested lead to strong activation of 4-1BB signal only when co-cultured with HER2 high expressing cell.

### 6.4. HER2 Quantitation

HER2 cell surface expression level was quantified on various cancer cell lines using QIFIKIT quantification kit(Dako) according to manufacturer's recommendation. Briefly, cells were stained with unlabeled anti-HER2 mouse monoclonal antibody(R&D systems) or purified mouse IgG2b isotype control (R&D systems) at saturating concentration. After washing, the stained cells and calibration beads from the kit were simultaneously labeled with the same FITC-conjugated goat anti-mouse IgG secondary antibody from the kit. Labeled cells and calibration beads were analyzed on a flow cytometer. A linear regression was performed using MFI values from the calibration beads. ABC (Antibody-Binding Capacity) was extrapolated from this regression line and sABC (specific ABC) was determined by subtracting ABC of the isotype control antibody from ABC of anti-HER2 antibody.

The obtained results are shown in Table 33.

TABLE 33

| Cell lines | | HER2 sABC |
|---|---|---|
| NCI-N87 | ATCC, CRL-5822 | 1,755,033 |
| Calu-3 | ATCC, HTB-55 | 734,348 |
| HCC1954 | ATCC, CRL-2338 | 497,805 |
| JIMT1 | DSMZ, ACC 589 | 93,113 |
| ZR-75-1 | ATCC, CRL-1500 | 25,360 |
| A431 | ATCC, CRL-1555 | 13,130 |
| MCF-7 | ATCC, HTB-22 | 8,525 |
| MDA-MB231 | ATCC, HTB-26 | 3,841 |
| BxPC-3 | ATCC, CRL-1687 | 2,013 |

As shown in Table 33, the sABC of 9 cancer cell lines was determined.

6.5. Correlation Between the HER2 sABC and 4-1BB-Induced NF-kB Signaling

The HER2 levels measured in Example 6.4 were standardized to HER2 levels expressed by HCC1954. The levels of 4-1BB activation by the bispecific antibody were determined as maximum level of fold change compared with control in 4-1BB NF-kB luciferase reporter assay of Example 6.2. Shared area indicates confidence interval for a linear fit.

The obtained results are shown in FIG. 7. As shown in FIG. 7, 4-1BB activation by anti-HER2/anti-4-1BB bispecific antibody showed a strong correlation with HER2 cell surface expression.

### Example 7. T Cell Immune Response 7.1. Effect on Release of Cytokine

To test the ability of bispecific antibodies to stimulated human peripheral blood mononuclear cells (PBMCs) response, the concentration of IFN-gamma in supernatant was measured. Human PBMCs were co-cultured with HCC1954 cancer cells expressing HER2 in the presence of anti-human CD3 antibody (BioLegend, 5 ug/mL) and the bispecific antibodies (Example 3; 3 ug/mL, 4-fold dilution) to be tested. After culture in a humidified chamber with 5% CO₂ at 37° C. for 72 hours, the concentration of IFN-gamma in supernatant was measured by Human IFN-gamma Quantikine Kit (R&D system, SIF50).

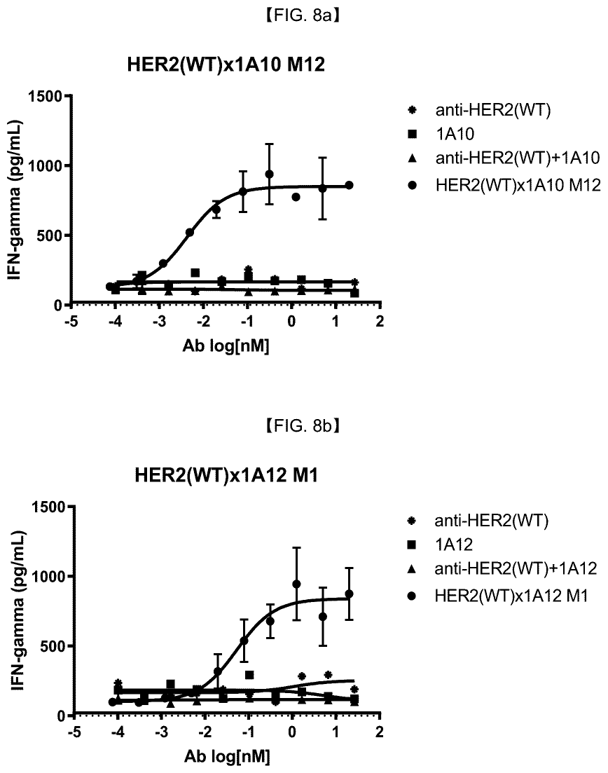

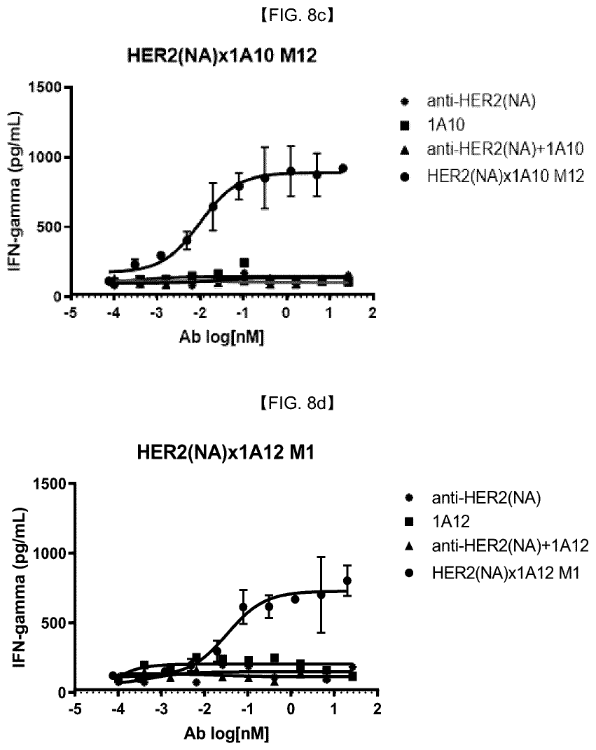

The obtained results are shown in FIGS. 8a-8d. As shown in FIGS. 8a-8d, all the tested bispecific antibodies induced cytokine release more than the combination of each monoclonal antibody in presence of HER2 high expressing cells.

### 7.2. Effect on Target Cell Growth

To test the ability of bispecific antibodies to stimulated human PBMCs response, target cell lysis assay was used. Human PBMCs were co-cultured with HCC1954 cancer cells expressing HER2 in the presence of anti-human CD3 antibody (BioLegend, 5ug/mL) and the bispecific antibodies (Example 3; 3 ug/mL, 4-fold dilution) to be tested. After culture in a humidified chamber with 5% CO₂ at 37° C. for 72 hours, the survival of HCC1954 was measured by cell counting kit-8 (Dojindo, CK04-20).

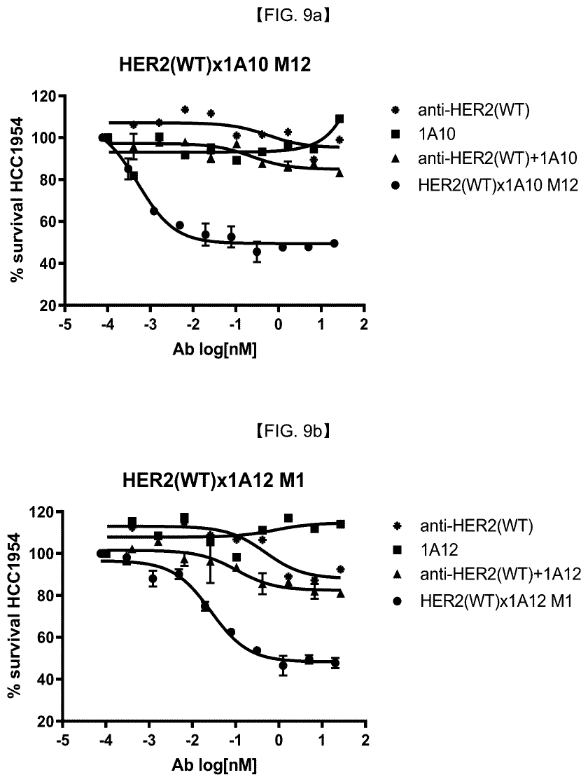

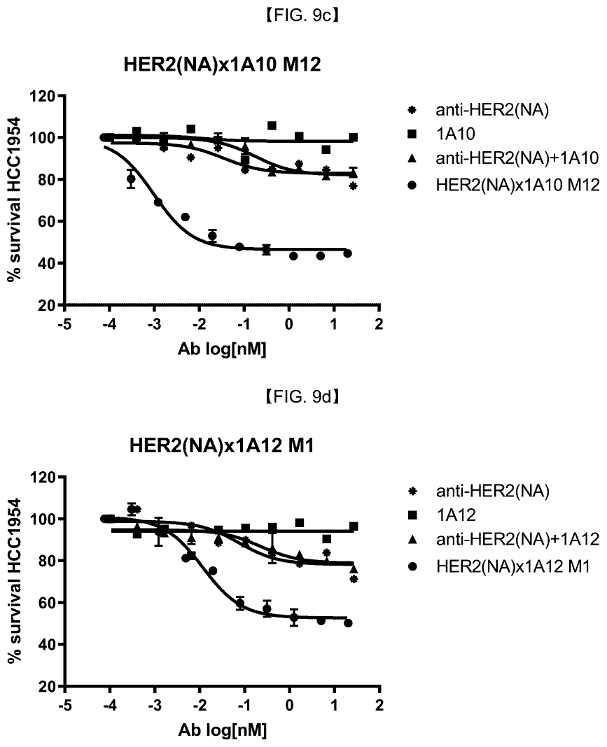

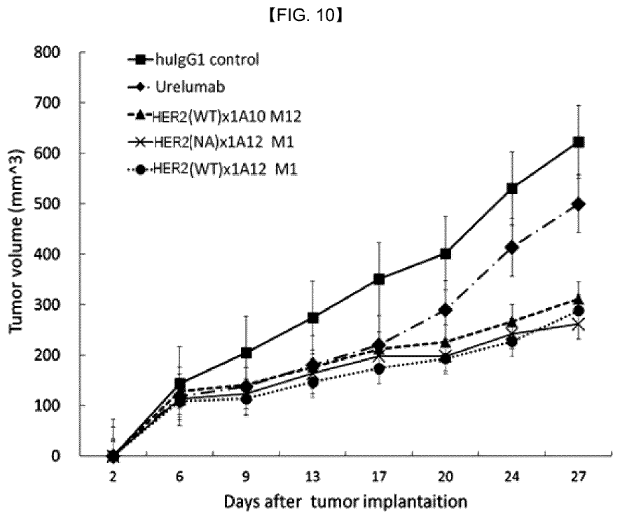

The obtained results are shown in FIGS. 9a-9d. As shown in FIGS. 9a-9d, all the tested bispecific antibodies showed superior cancer cell death activities compared to the combination of each monospecific antibody in presence of HER2 high expressing cells.

### Example 8. In Vivo Anti-Tumor Efficacy in HCC1954 Bearing hPBMC Engrafted Mice

### 8.1. Anti-Tumor Activity

To test in vivo anti-tumor efficacy of anti-HER2/anti-4-1BB bispecific antibodies, PBMC-humanized NSG mice were used. 7-week-old NSG mice (The Jackson Laboratory) were intravenously injected with 1×10⁷ human PBMC and HCC1954 cancer cells (1×10⁷ cells/mouse in PBS:Matrigel=1:1 solution) were inoculated into right flank of the mice. HCC1954 bearing humanized mice were assigned to each test group (n=12/group) at day 2 post tumor implantation. The mice were intravenously administrated with human IgG1 control antibody, anti-4-1BB antibody or anti-HER2/anti-4-1BB bispecific antibody twice a week at doses of 10 mg/kg or 7.5 mg/kg for 4 weeks. Antibodies were injected twice a week and the tumor size was measured with a digital caliper.

The obtained results are shown in FIG. 10. As shown in FIG. 10, all the tested bispecific antibodies showed superior anti-tumor activity to benchmark anti-4-1BB antibody (Urelumab, BMS) as well as the control antibody.

### 8.2. Analysis of Tumor-Infiltrating Lymphocytes (TIL)

To evaluate TIL, formalin-fixed, paraffin-embedded tumor tissue sections from HCC1954 bearing hPBMC engrafted mice were immunostained with anti-hCD45 antibody (human leukocyte marker, Cell Signaling Technology), anti-hCD4 antibody (human helper T-lymphocyte marker, Cell Signaling Technology), anti-hCD8 antibody (human cytotoxic T-lymphocyte marker, Cell Signaling Technology), and anti-hCD16 antibody (human natural killer cell marker, Cell Signaling Technology). The immunohistochemical technique was performed by applying the avidin-biotin detection kit (Vector Laboratories), to measure each marker positive cells. Briefly, Formalin-fixed paraffin-embedded tumoral tissue sections were deparaffinized and rehydrated. Antigen retrieval from rehydrated tissue sections were performed by placing sections in EDTA buffer (pH 9.0). After washing with phosphate-buffered saline (PBS) and incubation with blocking solution for 30 min, sections were incubated with primary antibodies at 4° C. overnight. Vectastain Elite ABC kit (Vector Lab) and the protocol provided by the manufacturer were used for the immunostaining. Sections were then counterstained with hematoxylin, dehydrated using graded alcohols and xylene, and mounted with Permount.

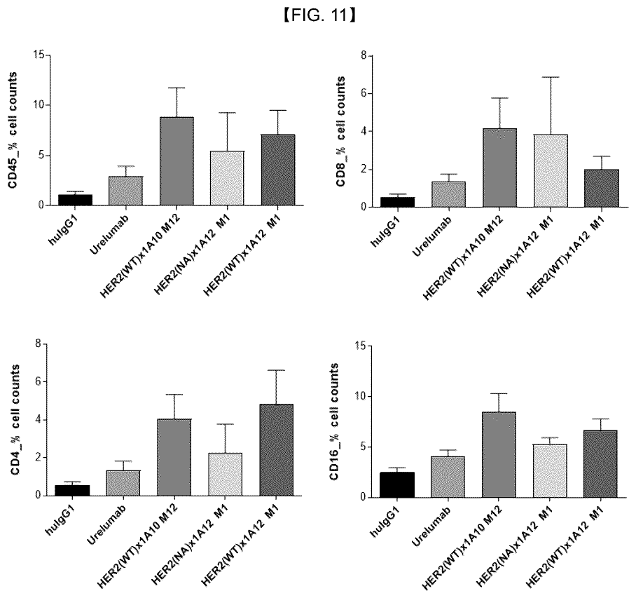

The obtained result is shown in FIG. 11. As shown in FIG. 11, Her2x41-BB bispecific antibodies effectively enhanced infiltration of immune cells including CD45⁺ cells, CD4⁺ T cells, CD8⁺ T cells, and NK cells into tumor tissues, compared to BMUR (Urelumab).

### Example 9. In Vivo Anti-Tumor Efficacy in 4-1BB Knock-In Mice

### 9.1. The Anti-Tumor Activity

In vivo anti-tumor efficacy of anti-HER2/anti-4-1BB bispecific antibodies was evaluated in human HER2/MC38 tumor (Biocytogen) bearing 4-1BB knock-in mice (Biocytogen). Tumor bearing humanized mice were randomized to each test group (n=5/group) at day 4 post tumor implantation based on tumor volume (approximately 80 mm³). Human IgG1 antibody, Trastuzumab (anti-HER2 antibody), and anti-HER2/anti-4-1BB bispecific antibodies (HER2 (WT)x1A10 M12, HER2 (NA)x1A10 M12) were respectively intra peritoneally administrated twice a week at dose of 10 mg/kg or 7.5 mg/kg into the mice for 4 weeks. Tumor size was measured with a digital caliper.

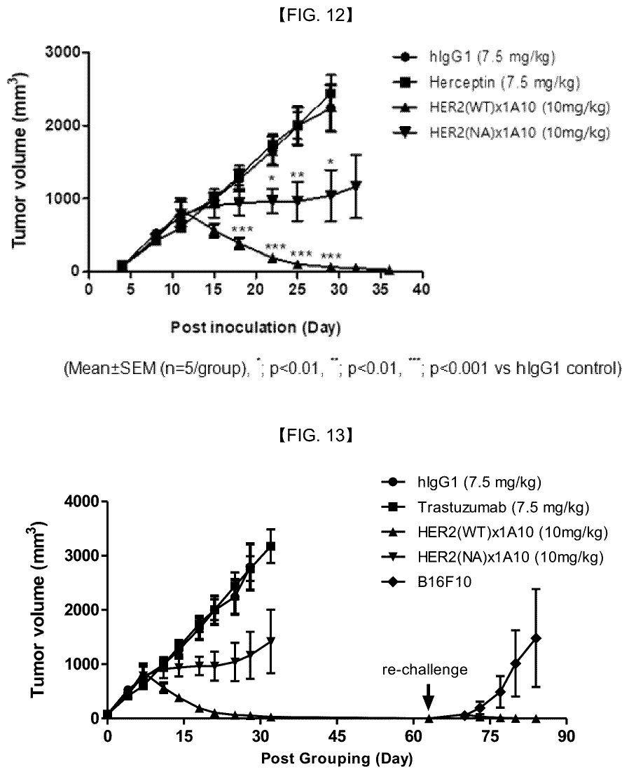

The obtained results are shown in FIG. 12. As shown in FIG. 12, anti-HER2/anti-4-1BB bispecific antibodies showed superior anti-tumor efficacy compared to Trastuzumab in human HER2/MC38 tumor. Especially, the tumors of all mice that were treated with HER2 (WT)x1A10 M12 have been cured.

### 9.2. Evaluation on the Effect of Tumor Specific Memory T Cell

The mice cured by HER2 (WT)x1A10 M12 were re-challenged with human HER2/MC38 tumor cells (Biocytogen) and B16 F10 tumor cells (ATCC) in both flanks at 63 days post tumor injection. Mice were not administered with any drug during re-challenge study period. Tumor size was measured with a digital caliper.

The obtained results are shown in FIG. 13. As shown in FIG. 13, human HER2/MC38 tumor development was not observed, whereas B16 F10 tumor was grown in mice cured by HER2 (WT)x1A10 M12 treatment.

### Example 10. Antibody-Dependent Cellular Cytotoxicity (ADCC) Activity (NA Backbone vs. WT)

### 10.1. NK Cell-Mediated ADCC

In this example, human peripheral blood-derived CD56⁺NK cells were used as effector cells and CellTrace Violet (Thermo Fisher Scientific)-labeled HCC1954 cells expressing HER2 were used as target cells. Cells were c o-cultured at an effector:target ratio of 5:1 with 50 nM of anti-HER2/anti-4-1BB bispecific antibodies (Example 3) at 37° C. After 4 hours, cells were stained with Fixable Viability Dye (eBioscience™) and then the ratio of dead target cells was analyzed by flow cytometry.

The obtained results are shown in following FIG. 14. As shown in FIG. 14, IgG1 type (WT) of anti-HER2/anti-4-1BB bispecific antibodies showed a prominent ADCC effect mediated by NK cells.

### 10.2. 4-1BB Signal Activation Dependent on FcγRIIb Engagement

In this example, CHO-K1 cells expressing FcγRIIb (Promega) were plated in a 96-well assay plate and cultured overnight. On the day of assay, Jurkat/4-1BB cells (Promega) were plated in 96 well plate. Cells were incubated with a titration of anti-HER2/anti-4-1BB bispecific antibodies in the presence (FcγRIIb dependent) or absence (FcγRIIb independent) of CHO-K1 cells expressing FcγRIIb (Promega). After 6 hours of induction, Bio-Glo™ Luciferase Assay reagent was added and luminescence was determined using a SpectraMax L luminometer (Molecular Devices). Four-parameter logistic curve analysis was performed with GraphPad Prism® software.

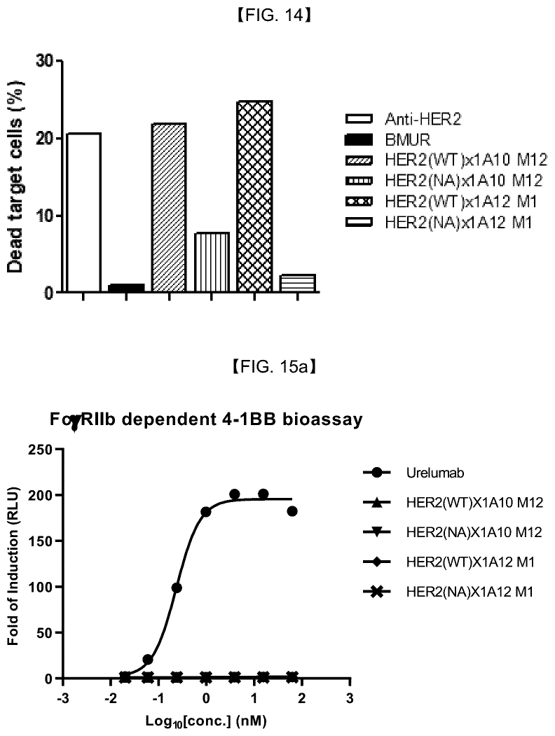

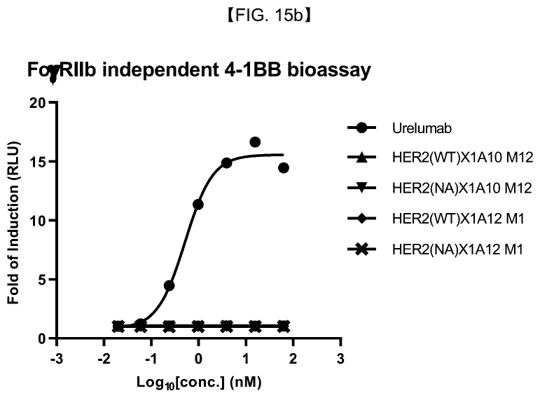

The obtained results are shown in following Tables 34 (FcγRIIb-dependent 4-1BB bioassay) and 35 (FcγRIIb-independent 4-1BB bioassay), and FIGS. 15a (FcγRIIb-dependent 4-1BB bioassay) and 15b (FcγRIIb-independent 4-1BB bioassay).

TABLE 34
FcγRIIb-dependent 4-1BB bioassay

| | Fold of induction (RLU) | EC₅₀ (nM) |
|---|---|---|
| Urelumab | 195.5 | 0.2364 |
| HER2(WT)X1A10 M12 | <2 | Not applicable |
| HER2(NA)X1A10 M12 | <2 | Not applicable |
| HER2(WT)X1A12 M1 | <2 | Not applicable |
| HER2(NA)X1A12 M1 | <2 | Not applicable |

TABLE 35
FcγRIIb-independent 4-1BB bioassay

| | Fold of induction (RLU) | EC₅₀ (nM) |
|---|---|---|
| Urelumab | 15.57 | 0.5196 |
| HER2(WT)X1A10 M12 | <2 | Not applicable |
| HER2(NA)X1A10 M12 | <2 | Not applicable |
| HER2(WT)X1A12 M1 | <2 | Not applicable |
| HER2(NA)X1A12 M1 | <2 | Not applicable |

As shown in Tables 34 and 35, and FIGS. 15a and 15b, Urelumab-treated group showed 13.5-fold difference in top RLU and 2.2-fold difference in EC₅₀ according to the presence of FcγRIIb CHO-K1 cells. Four anti-HER2/anti-4-1BB bispecific antibodies showed very low RLU compared to Urelumab regardless of the presence or absence of FcγRIIb CHO-K1 cells. These data showed that all the tested anti-HER2/anti-4-1BB bispecific antibodies have potential benefits compared to urelumab, which has severe toxicity in clinical studies (NCT00309023, NCT00612664, NCT014712210).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and “one or more” and similar referents in the context of describing the invention (especially in the context of the following claims) are to be construed to cover both the singular and the plural, unless otherwise indicated herein or clearly contradicted by context. The use of the term “at least one” (or “one or more”) followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “comprising, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method for treating a cancer in a subject in need thereof, the method comprising administering to the subject a therapeutically effective amount of an anti-4-1BB/anti-HER2 bispecific antibody,
wherein the anti-4-1BB/anti-HER2 bispecific antibody comprises:
(a) an anti-4-1BB antibody or an antigen-binding fragment thereof, and
(b) an anti-HER2 antibody or an antigen-binding fragment thereof,
wherein the anti-4-1BB antibody or an antigen-binding fragment thereof comprises:
(1) an H-CDR1 comprising an amino acid sequence of SEQ ID NO:1,
an H-CDR2 comprising an amino acid sequence of SEQ ID NO:4,
an H-CDR3 comprising an amino acid sequence of SEQ ID NO:7,
an L-CDR1 comprising an amino acid sequence of SEQ ID NO:12,
an L-CDR2 comprising an amino acid sequence of SEQ ID NO:14, and
an L-CDR3 comprising an amino acid sequence of SEQ ID NO:16;
(2) an H-CDR1 comprising an amino acid sequence of SEQ ID NO:1,
an H-CDR2 comprising an amino acid sequence of SEQ ID NO:4,
an H-CDR3 comprising an amino acid sequence of SEQ ID NO:8,
an L-CDR1 comprising an amino acid sequence of SEQ ID NO:12,
an L-CDR2 comprising an amino acid sequence of SEQ ID NO:14, and
an L-CDR3 comprising an amino acid sequence of SEQ ID NO:16;
(3) an H-CDR1 comprising an amino acid sequence of SEQ ID NO:1,
an H-CDR2 comprising an amino acid sequence of SEQ ID NO:4,
an H-CDR3 comprising an amino acid sequence of SEQ ID NO:9,
an L-CDR1 comprising an amino acid sequence of SEQ ID NO:12,
an L-CDR2 comprising an amino acid sequence of SEQ ID NO:14, and
an L-CDR3 comprising an amino acid sequence of SEQ ID NO:16;
(4) an H-CDR1 comprising an amino acid sequence of SEQ ID NO:2,
an H-CDR2 comprising an amino acid sequence of SEQ ID NO:5,
an H-CDR3 comprising an amino acid sequence of SEQ ID NO:10,
an L-CDR1 comprising an amino acid sequence of SEQ ID NO:12,
an L-CDR2 comprising an amino acid sequence of SEQ ID NO:14, and
an L-CDR3 comprising an amino acid sequence of SEQ ID NO:16; or
(5) an H-CDR1 comprising an amino acid sequence of SEQ ID NO:3,
an H-CDR2 comprising an amino acid sequence of SEQ ID NO:6,
an H-CDR3 comprising an amino acid sequence of SEQ ID NO:11,
an L-CDR1 comprising an amino acid sequence of SEQ ID NO:13,
an L-CDR2 comprising an amino acid sequence of SEQ ID NO:15, and
an L-CDR3 comprising an amino acid sequence of SEQ ID NO:17.

2. The method according to claim 1, wherein
the anti-4-1BB antibody or the antigen-binding fragment of claim 1 (1) comprises a heavy chain variable region comprising an amino acid sequence of SEQ ID NO:18 and a light chain variable region comprising an amino acid sequence of SEQ ID NO:30, or
a heavy chain variable region comprising an amino acid sequence of SEQ ID NO:24 and a light chain variable region comprising an amino acid sequence of SEQ ID NO:33;
the anti-4-1BB antibody or the antigen-binding fragment of claim 1 (2) comprises a heavy chain variable region comprising an amino acid sequence of SEQ ID NO:19 and a light chain variable region comprising an amino acid sequence of SEQ ID NO:30 or 31, or
a heavy chain variable region comprising an amino acid sequence of SEQ ID NO:25 and a light chain variable region comprising an amino acid sequence of SEQ ID NO:33 or 34;
the anti-4-1BB antibody or the antigen-binding fragment of claim 1 (3) comprises a heavy chain variable region comprising an amino acid sequence of SEQ ID NO:20 and a light chain variable region comprising an amino acid sequence of SEQ ID NO:30 or 31;
the anti-4-1BB antibody or the antigen-binding fragment of claim 1 (4) comprises a heavy chain variable region comprising an amino acid sequence of SEQ ID NO:21 and a light chain variable region comprising an amino acid sequence of SEQ ID NO:30,
a heavy chain variable region comprising an amino acid sequence of SEQ ID NO:22 and a light chain variable region comprising an amino acid sequence of SEQ ID NO:31,
a heavy chain variable region comprising an amino acid sequence of SEQ ID NO:27 and a light chain variable region comprising an amino acid sequence of SEQ ID NO:33, or
a heavy chain variable region comprising an amino acid sequence of SEQ ID NO:28 and a light chain variable region comprising an amino acid sequence of SEQ ID NO:34; or
the anti-4-1BB antibody or the antigen-binding fragment of claim 1 (5) comprises a heavy chain variable region comprising an amino acid sequence of SEQ ID NO:23 and a light chain variable region comprising an amino acid sequence of SEQ ID NO:32, or
a heavy chain variable region comprising an amino acid sequence of SEQ ID NO:29 and a light chain variable region comprising an amino acid sequence of SEQ ID NO:88.

3. The method according to claim 1, wherein
the anti-4-1BB antibody or the antigen-binding fragment of claim 1 (1) comprises a heavy chain comprising an amino acid sequence of SEQ ID NO:56; and a light chain comprising an amino acid sequence of SEQ ID NO:62;
the anti-4-1BB antibody or the antigen-binding fragment of claim 1 (2) comprises a heavy chain comprising an amino acid sequence of SEQ ID NO:57; and a light chain comprising an amino acid sequence of SEQ ID NO:62 or 63;
the anti-4-1BB antibody or the antigen-binding fragment of claim 1 (3) comprises a heavy chain comprising an amino acid sequence of SEQ ID NO:58; and a light chain comprising an amino acid sequence of SEQ ID NO:62 or 63;
the anti-4-1BB antibody or the antigen-binding fragment of claim 1 (4) comprises a heavy chain comprising an amino acid sequence of SEQ ID NO:59; and a light chain comprising an amino acid sequence of SEQ ID NO:62, or
a heavy chain comprising an amino acid sequence of SEQ ID NO:60; and a light chain comprising an amino acid sequence of SEQ ID NO:63; or
the anti-4-1BB antibody or the antigen-binding fragment of claim 1 (5) comprises a heavy chain comprising an amino acid sequence of SEQ ID NO:61; and a light chain comprising an amino acid sequence of SEQ ID NO:64.

4. The method according to claim 1,
wherein the anti-4-1BB antibody or an antigen-binding fragment thereof is an anti-4-1BB scFv of an anti-4-1BB antibody.

5. The method according to claim 4,
wherein the anti-4-1BB scFv further comprises a peptide linker between the heavy chain variable region and the light chain variable region.

6. The method according to claim 1,
wherein the anti-HER2 antibody or an antigen-binding fragment thereof is Trastuzumab, Pertuzumab, or Trastuzumab emtansine (T-DM1).

7. The method according to claim 1,
wherein the anti-HER2 antibody or an antigen-binding fragment thereof comprises:
an H-CDR1 comprising an amino acid sequence of SEQ ID NO:65;
an H-CDR2 comprising an amino acid sequence of SEQ ID NO:66;
an H-CDR3 comprising an amino acid sequence of SEQ ID NO:67;
an L-CDR1 comprising an amino acid sequence of SEQ ID NO:68;
an L-CDR2 comprising an amino acid sequence of SEQ ID NO:69; and
an L-CDR3 comprising an amino acid sequence of SEQ ID NO:70.

8. The method according to claim 1,
wherein the anti-HER2 antibody or an antigen-binding fragment thereof comprises:
a heavy chain comprising an amino acid sequence of SEQ ID NO:73, or 74; and
a light chain comprising an amino acid sequence of SEQ ID NO:75.

9. The method according to claim 1,
wherein the anti-4-1BB antibody or an antigen-binding fragment thereof is an anti-4-1BB scFv of an anti-4-1BB antibody, and
wherein the anti-HER2 antibody or an antigen-binding fragment thereof is a full-length form of an anti-HER2 antibody.

10. The method according to claim 9,
wherein the anti-4-1BB/anti-HER2 bispecific antibody further comprises a peptide linker between the anti-4-1BB scFv and the heavy chain of the full-length form of an anti-HER2 antibody.

11. The method according to claim 10,
wherein the peptide linker comprises an amino acid sequence of SEQ ID NO:85 or 86.

12. The method according to claim 1,
wherein the anti-4-1BB/anti-HER2 bispecific antibody comprises:
a polypeptide comprising an amino acid sequence of SEQ ID NO:76, 77, 78, 79, 80, 81, 82, 83, or 84; and
a polypeptide comprising an amino acid sequence of SEQ ID NO:75.

13. The method according to claim 1,
wherein the anti-4-1BB/anti-HER2 bispecific antibody comprises:
a polypeptide comprising an amino acid sequence of SEQ ID NO:83; and
a polypeptide comprising an amino acid sequence of SEQ ID NO:75.

14. The method according to claim 1,
wherein the cancer is characterized by HER2 expression.
